(19) United States
(12) Patent Application Publication
(10) Pub. No.: US 2025/0320521 A1
(43) Pub. Date: Oct. 16, 2025

(54) ERYTHROPARVOVIRUS COMPOSITIONS AND METHODS FOR GENE THERAPY

(71) Applicant: Synteny Therapeutics, Inc., Cambridge, MA (US)

(72) Inventors: Robert Kotin, Cambridge, MA (US); Sebastian Aguirre Kozlouski, Cambridge, MA (US)

(21) Appl. No.: 18/864,130

(22) PCT Filed: May 9, 2023
(86) PCT No.: PCT/US23/21496

§ 371 Date: Nov. 8, 2024

Related U.S. Application Data
(60) Provisional application No. 63/339,587, filed on May 9, 2022.
(60) Provisional application No. 63/339,742, filed on May 9, 2022.

(51) Int. Cl. C12N 15/86; A61K 48/00; C07K 14/005

(52) U.S. Cl. CPC C12N 15/86; A61K 48/005; C07K 14/005; C12N 2710/14043; C12N 2750/14222; C12N 2750/14243; C12N 2750/14251; C12N 2800/40

(57) ABSTRACT

Disclosed are recombinant virions that have a capsid protein or a variant thereof of erythroparvovirus and a nucleic acid that includes a heterologous nucleic acid.

### CROSS REFERENCE TO RELATED APPLICATIONS

This application claims the benefit of U.S. Application Ser. No. 63/339,587 filed on May 9, 2022, and U.S. Application Ser. No. 63/339,742 filed on May 9, 2022, the disclosures of each of which are hereby incorporated by reference in their entireties.

### BACKGROUND

Recombinant viral particles (or recombinant virions) are commonly utilized for gene therapy. The present disclosure provides technologies relating to erythroparvovirus compositions comprising at least one erythroparvovirus capsid protein, and their production and use, including in gene therapy.

### SUMMARY OF INVENTION

The present disclosure recognizes a need for improvements in gene therapy technologies. For example, among other things, the present disclosure recognizes a need for improved compositions, preparations, recombinant virions, host cells, etc. Furthermore, the present disclosure specifically recognizes a need for improved production and manufacturing of recombinant virions that comprise or otherwise utilize at least one erythroparvovirus capsid protein.

The present disclosure is based, at least in part, on the discovery that a recombinant virion comprising at least one capsid protein of an erythroparvovirus is particularly advantageous as a vehicle for gene therapy. First, due to a larger virion genome size (˜5.6 kb compared with ˜4.7 kb of AAV), an erythroparvovirus can package a nucleic acid at least 1 kb greater than AAV, thereby allowing delivery of therapeutic genes whose size exceeds the capacity of AAV. A larger virion genome size also allows delivery of a therapeutic transgene(s) together with genomic safe harbor (GSH) sequences that accommodate site-specific recombination of a transgene(s) at a desired genomic location. Such site-specific recombination allows integration of a transgene at an inert location in a genome, as opposed to random integration that could disrupt an essential gene and its expression. Second, unlike AAV, erythroparvovirus does not appear to be as prevalent as AAV. Thus, administration of an erythroparvovirus, e.g., comprising a therapeutic gene, would not trigger an extensive anti-viral immune reaction that precludes efficient gene delivery. Accordingly, erythroparvovirus can achieve gene delivery with an efficiency unparalleled to AAV. Third, erythroparvovirus has an extraordinary tropism for hematopoietic cells which makes it particularly attractive for use in preventing or treating hematologic diseases including but not limited to hemoglobinopathies, anemia, hemophilia, myeloproliferative disorders, coagulopathies, and cancer.

While attempts have been made to utilize erythroparvovirus as a vehicle for gene therapy, such attempts have not been successfully developed. Notably, transduction efficiency was low and not feasible for clinical use. For example, transduction of erythroparvovirus B19 lacked correlation with the presence and/or amount of P-antigen, a cell surface marker that erythroparvovirus B19 binds, which questioned its specificity and its utility for targeting cells (e.g., hematopoietic cells). Thus, compositions, preparations, recombinant virions, host cells, and methods of using same presented herein represent new approaches that transform gene therapy targeting cells (e.g., hematopoietic cells).

Among other things, in some embodiments, provided herein are recombinant virions comprising at least one capsid protein (or a variant thereof) of an erythroparvovirus or a pharmaceutical composition comprising said recombinant virions. In some embodiments, provided herein are recombinant virions comprising at least one capsid protein (or a variant thereof) of an erythroparvovirus B19 or a pharmaceutical composition comprising said recombinant virions. Also, in some embodiments, provided herein are recombinant virions having homology arms (e.g., sequences with homology to the genomic DNA of a target cell) that can facilitate integration of a heterologous nucleic acid into a specific site within a target genome, and methods of integrating said nucleic acid within the target genome. In some embodiments, integration is mediated by cellular processes, such as homologous recombination or non-homologous end joining. In some embodiments, integration is initiated and facilitated by an exogenously introduced nuclease (e.g., ZFN, TALEN, CRISPR/Cas9-gRNA).

Among other things, in some embodiments, also provided herein are methods of preventing or treating a disease in a subject using recombinant virions described herein. In some embodiments, recombinant virions are administered to the subject, thereby preventing or treating the disease in vivo. In some embodiments, a method comprises obtaining a plurality of cells from a subject, transducing recombinant virions described herein, and administering an effective amount of transduced cells to the subject. For example, in some embodiments, a high affinity and specificity of erythroparvoviral capsid protein(s) for hematopoietic cells make the described recombinant virions particularly useful in gene therapy for hematological diseases (e.g., hemoglobinopathies). In some embodiments, methods further comprise re-administering an additional amount of a recombinant virion, a pharmaceutical composition, or transduced cells (e.g., for repeat dosing after an attenuation or for calibration).

Among other things, in some embodiments, a nucleic acid of recombinant virions and/or pharmaceutical compositions encodes a protein, e.g., a therapeutic protein. In some embodiments, a nucleic acid decreases or eliminates expression of an endogenous gene (e.g., via RNAi, CRISPR, etc.).

Among other things, in some embodiments, the present disclosure provides use of recombinant virions and/or pharmaceutical compositions for treatment or prevention of a disease of a subject. In some embodiments, the present disclosure provides use of a recombinant virions and/or pharmaceutical compositions described herein for preparation of a medicament for preventing or treating a subject (e.g., human) in need thereof.

Among other things, in some embodiments, provided herein are methods of modulating gene expression in a cell or a subject, comprising transducing recombinant virions and/or pharmaceutical compositions described herein. Such modulation may involve increasing or restoring the expression of an endogenous gene whose expression is aberrantly lower than the expression in a healthy subject. Alternatively, modulation may involve decreasing or eliminating expression of an endogenous gene whose expression is aberrantly higher than expression in a healthy subject.

Among other things, in some embodiments, provided herein are methods of modulating a function and/or structure of a protein in a target cell, whose function and/or structure is different from the wild-type protein (e.g., due to a mutation or aberrant gene expression). In certain embodiments, said modulation may improve and/or restore the function and/or structure of a defective protein in a cell of a subject afflicted with a disease. In some such embodiments, said method of modulating the function and/or structure of a protein improves and/or restores the function and/or structure of hemoglobin in a cell of a subject afflicted with sickle cell anemia.

Among other things, in some embodiments, provided herein are methods and compositions for producing recombinant virions and/or pharmaceutical compositions described herein. In some embodiments, recombinant virions are produced in mammalian cells by introducing a set of genes that express virus structural and non-structural proteins and a virion genome. In preferred embodiments, recombinant virions and/or pharmaceutical compositions are produced by infecting host cells (e.g, insect cells, e.g., mammalian cells). In certain embodiments, a nucleic acid comprising a sequence for producing virions (e.g., a nucleic acid comprising at least one ITR sequence or origin of virion DNA replication, a nucleic acid encoding at least one viral replication protein, a nucleic acid encoding at least one erythroparvovirus capsid protein, e.g., at least one Erythroparvovirus B19 capsid protein) is introduced into mammalian cells transiently. In certain embodiments, a nucleic acid comprising a sequence for producing virions (e.g., a nucleic acid comprising at least one ITR sequence or origin of virion DNA replication, a nucleic acid encoding at least one viral replication protein, a nucleic acid encoding at least one erythroparvovirus capsid protein (e.g., at least one erythroparvovirus B19 capsid protein) is introduced into insect cells transiently. In some embodiments, a nucleic acid is integrated within a mammalian cell genome. In some embodiments, a nucleic acid is integrated within an insect cell genome.

### BRIEF DESCRIPTION OF FIGURES

FIG. 1A and FIG. 1B show a secondary structure of AAV ITR and a schematic diagram of a rolling hairpin replication model, according to some embodiments of the present disclosure. FIG. 1A shows a structure of AAV ITR that forms an extensive secondary structure. An ITR can acquire two configurations (flip and flop). FIG. 1B shows a schematic diagram showing a rolling hairpin replication model by which a viral nucleic acid replicates.

FIG. 2A-FIG. 2E each shows a map of nucleic acids encoding VP1 capsid protein variants (VP1-TTG; VP1-CTG; VP1-ACG), nonstructural protein (NS), and an exemplary vector comprising a nucleic acid encoding VP1-TTG of human erythroparvovirus B19, according to some embodiments of the present disclosure.

FIG. 3 shows schematic diagrams representing a heterologous nucleic acid/a transgene construct containing a β-globin gene operably linked to a β-globin promoter flanked at the 5′ terminus by one or more HS sequences, according to some embodiments of the present disclosure. Mammalian β-globin gene is regulated by a regulatory region called the locus control region (LCR) containing a series of 5 DNase I hypersensitive sites (HS1-HS5). The HSs is required for efficient expression of the β-globin gene. Each transgene construct is placed between two homology arms (a 5′ homology arm and a 3′ homology arm), which facilitates site-specific integration at a target cell genome by homologous recombination.

FIG. 4 shows schematic diagrams representing a heterologous nucleic acid/a transgene construct containing various promoters. Each promoter (e.g., CAG promoter, AHSP promoter, MND promoter, W-A promoter, PKLR promoter) is operably linked to a transgene of interest, and the entire construct is placed between two homology arms (a 5′ homology arm and a 3′ homology arm), which facilitates site-specific integration at a target cell genome by homologous recombination, according to some embodiments of the present disclosure.

FIG. 5 shows partial DNA sequence of the erythroid-specific promoter of PKLR, according to some embodiments of the present disclosure. A 469-bp region comprising the upstream regulatory domain. Conserved elements between the human and rat PK-R promoter are depicted by dotted lines. The cytosine of the PK-R transcriptional start site is underlined. GATA-1, CAC/Spl motifs, and the regulatory element PKR-REI in the upstream 270-bp region are shown in boxes (orientation indicated by arrows).

FIG. 6A and FIG. 6B show exemplary miRNAs that can be targeted by recombinant virions described herein. Erythroparvovirus recombinant virions may comprise the miRNA sequences. Alternatively, recombinant virions may comprise a nucleic acid sequence that inactivates the miRNAs.

FIG. 7 depicts exemplary methods and infection conditions for production of recombinant virions comprising an exemplary erythroparvovirus B19 VP1 capsid, according to some embodiments of the present disclosure. Production methods may comprise triple infection (e.g., AAV genome, capsid, rep) or double infection (e.g., AAV genome, rep/cap). Infection conditions may comprise a culture volume of 200 ml, an Sf9 cells density of 2.5E+6 cells/ml, and a Baculovirus Infected Insect Cell (BIIC) dilution of 1:10,000 as described herein.

FIG. 8 shows a line graph depicting a total number of Sf9 cells infected with recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and recombinant virions comprising an AAV2 VP1 capsid protein encoded by a nucleotide sequence according to SEQ ID NO: 35 (Exemplary AAV2 Construct 1) at 24, 48, 72, 96, and 120 hours post infection (hpi).

FIG. 9 shows a line graph depicting cell viability Sf9 cells infected with recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and recombinant virions comprising an AAV2 VP1 capsid protein encoded by a nucleotide sequence according to SEQ ID NO: 35 (Exemplary AAV2 Construct 1), at 24, 48, 72, 96, and 120 hours post infection (hpi).

FIG. 10 shows a line graph depicting average cell diameter of Sf9 cells infected with recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and recombinant virions comprising an AAV2 VP1 capsid protein encoded by a nucleotide sequence according to SEQ ID NO: 35 (Exemplary AAV2 Construct 1), at 24, 48, 72, 96, and 120 hours post infection (hpi).

FIG. 11 shows a line graph depicting percent GFP-positive Sf9 cells infected with recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and recombinant virions comprising an AAV2 VP1 capsid protein encoded by a nucleotide sequence according to SEQ ID NO: 35 (Exemplary AAV2 Construct 1), at 24, 48, 72, 96, and 120 hours post infection (hpi).

FIG. 12 shows rescue of an AAV2 genome in cells infected with recombinant virions comprising an exemplary erythroparvovirus B19 VP1 capsid protein encoded by a nucleotide sequence an according to SEQ ID NOs: 29-32 (Exemplary B19 Construct 1, Exemplary B19 Construct 2, Exemplary B19 Construct 3, Exemplary B19 Construct 4, respectively) and cells infected with virions comprising an AAV2 VP1 capsid protein encoded by a nucleotide sequence according to SEQ ID NO: 35 (Exemplary AAV2 Construct 1) via PCR analysis.

FIG. 13 depicts crude virion yields (vg/ml and vg/cell) for recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and for recombinant virions comprising an AAV2 VP1 capsid protein encoded by a nucleotide sequence according to SEQ ID NO: 35 (Exemplary AAV2 Construct 1).

FIG. 14 shows virion density across different fraction collections for recombinant virions comprising an exemplary erythroparvovirus B19 VP1 capsid protein encoded by a nucleotide sequence according to SEQ ID NO: 29 (Exemplary B19 Construct 1).

FIG. 15 shows virion density across different fraction collections for recombinant virions comprising an exemplary erythroparvovirus B19 VP1 capsid protein encoded by a nucleotide sequence according to SEQ ID NO: 30 (Exemplary B19 Construct 2).

FIG. 16 shows virion density across different fraction collections for recombinant virions comprising an exemplary erythroparvovirus B19 VP1 capsid protein encoded by a nucleotide sequence according to SEQ ID NO: 31 (Exemplary B19 Construct 3).

FIG. 17 shows virion density across different fraction collections for recombinant virions comprising an exemplary erythroparvovirus B19 VP1 capsid protein encoded by a nucleotide sequence according to SEQ ID NO: 32 (Exemplary B19 Construct 4).

FIG. 18 shows virion density across different fraction collections for virions comprising an exemplary AAV2 VP1 capsid protein encoded by a nucleotide sequence according to SEQ ID NO: 35 (Exemplary AAV2 Construct 1).

FIGS. 19A-19B show a western blot analysis using an anti-VP2 capsid protein specific antibody of ultra-centrifuged (UC)-purified cell fractions. FIG. 19A shows the presence of erythroparvovirus B19 VP1 and VP2 capsid proteins in crude lysates of cells infected with recombinant virions comprising an erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively). FIG. 19B shows the presence of erythroparvovirus B19 VP1 and VP2 capsid proteins in crude lysates (left) and purified virions (right) from cells infected with recombinant virions comprising an erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively). VP1 and VP2 capsid proteins were detected in crude lysates of cells infected with recombinant virions comprising an erythroparvovirus B19 VP1 capsid protein encoded by a nucleotide sequence according to SEQ ID NOs: 29-31 (Exemplary B19 Construct 1, Exemplary B19 Construct 2, Exemplary B19 Construct 3). A faint VP2 capsid protein band was observed in crude lysates and UC-purified fractions of cells infected with recombinant virions comprising an erythroparvovirus B19 VP1 capsid protein encoded by a nucleotide sequence according to SEQ ID NO: 32 (Exemplary B19 Construct 4).

FIG. 20 shows fluorescence (top) and phase imaging (bottom) of transduction of recombinant virions comprising an exemplary erythroparvovirus B19 VP1 capsid protein encoded by an exemplary nucleotide sequence according to SEQ ID NOs: 29-32 (Exemplary B19 Construct 1, Exemplary B19 Construct 2, Exemplary B19 Construct 3, Exemplary B19 Construct 4, respectively) and a heterologous nucleic acid encoding GFP in K562 cells.

FIG. 21 shows a bar graph depicting percent GFP-positive K562 cells infected with recombinant virions comprising an exemplary erythroparvovirus B19 VP1 capsid protein encoded by a nucleotide sequence according to SEQ ID NO: 30 (Exemplary B19 Construct 2).

### DETAILED DESCRIPTION OF THE INVENTION

Efficient delivery of a therapeutic transgene is a prerequisite for successful gene therapy. When gene therapy was conceptualized in the early 1970s, mammalian viruses were proposed as an effective vehicle to deliver a gene ‘drug.’ Since then, viral vectors have been intensively investigated and broadly used in gene transfer applications. In recent years, adeno-associated virus (AAV) has emerged as a preferred viral vector for gene therapy due to its ability to transduce a wide range of cell types, cross the blood-brain-barrier, and maintain long-term stable expression predominantly as an episomal element. AAV vectors, derived from the non-pathogenic dependoparvovirus genus of the Parvovirus family, retain no virus genes and have been developed for human applications with relatively few reports of vector related serious adverse events.

However, certain characteristics of AAV impose limitations to its application to gene therapy. In particular, AAV is only capable of packaging less than 5 kb of therapeutic DNA, excluding many therapeutic genes and approaches from development. For example, a therapeutic gene for treating the serious genetic diseases with the greatest incidence, namely Duchenne muscular dystrophy, hemophilia A, and cystic fibrosis, exceeds the size limitation of AAV, thus excluding AAV-mediated gene therapy as a treatment option for these diseases. Moreover, the fact that many AAV serotypes appear to be endemic results in extensive anti-viral immunity in human populations, complicating AAV gene transfer in many subjects. The prevalence of seroconversion to AAVs has been estimated as greater than 70% in adults. Seroconversion typically occurs in childhood due to a productive (co-) infection with a wild-type AAV and helper virus, often adenovirus, generating antibodies that cross-react with epitopes common to most primate AAV capsids. Currently, prospective gene therapy patients are screened for neutralizing antibodies (nAbs) and may be ineligible for AAV gene therapy if nAbs exceed an arbitrarily selected threshold titer. Thus, a large portion of patient population is excluded from gene therapy by AAV. Furthermore, although the natural diversity of AAVs is vast, and host tropism differs among AAV species, several important cell types and tissues for gene therapy remain to be unlocked for targeting.

Accordingly, there is a great need for viral compositions and methods for gene therapy that incorporate the utility of AAV vectors while overcoming the limitations.

Moreover, in some embodiments, provided herein are recombinant virions, pharmaceutical compositions, and methods that allow efficient gene therapy.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow a therapy to perform its intended function. Examples of routes of administration include injection (intramuscular, subcutaneous, intravenous, parenterally, intraperitoneally, intrathecal, intranasal, intracranial, intravitreal, subretinal, etc.) routes. The routes of administration also include direct injection to the bone marrow. The injection can be a bolus injection or can be a continuous infusion. Depending on the route of administration, the agent can be coated with or disposed in a selected material to protect it from natural conditions which may detrimentally affect its ability to perform its intended function.

The term “gene” is used broadly to refer to any nucleic acid associated with a biological function. The term “gene” applies to a specific genomic sequence, as well as to a cDNA or an mRNA encoded by that genomic sequence. Genes can be associated with regulatory elements, such as enhancers, promoters, and locus control regions, untranslated regions (UTRs), introns, polyadenylation signals, Kozak motifs, TATA-boxes or TATA-less promoters, and post-transcriptional elements, e.g., WPRE.

The term “heterologous” is art-recognized, and when used in relation to a nucleic acid in a recombinant virion, a heterologous nucleic acid is heterologous to the virus from which the at least one capsid protein originates.

The term “homologous recombination” is art-recognized, and when used in relation to a nucleic acid insertion in a target genome, it is intended to include homology-dependent repair.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12 (I): 387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). The term “subject” or “patient” refers to any healthy or diseased animal, mammal or human, or any animal, mammal or human. In some embodiments, the subject is afflicted with a hematologic disease. In various embodiments of the methods of the present invention, the subject has not undergone treatment. In other embodiments, the subject has undergone treatment.

A “therapeutically effective amount” of a substance or cells or virions is an amount capable of producing a medically desirable result (e.g., clinical improvement) in a treated patient with an acceptable benefit: risk ratio, preferably in a human or non-human mammal.

The term “treating” includes prophylactic and/or therapeutic treatments. The term “prophylactic or therapeutic” treatment is art-recognized and includes administration to the subject one or more of the compositions described herein. If it is administered prior to clinical manifestation of the unwanted condition (e.g., disease or other unwanted state of the subject), then the treatment is prophylactic (i.e., it protects the subject against developing the unwanted condition); whereas, if it is administered after manifestation of the unwanted condition, the treatment is therapeutic (i.e., it is intended to diminish, ameliorate, or stabilize the existing unwanted condition or side effects thereof).

### Recombinant Virion

Among other things, provided herein are recombinant virions, pharmaceutical compositions, and methods that allow efficient gene therapy.

For example, in some embodiments, provided herein are recombinant virions comprising at least one capsid protein of erythroparvovirus (e.g., erythroparvovirus B19) and a nucleic acid comprising a heterologous nucleic acid. In some embodiments, a recombinant virion comprises all capsid proteins of erythroparvovirus (e.g., erythroparvovirus B19). In some embodiments, a recombinant virion comprises a capsid of an erythroparvovirus (e.g., erythroparvovirus B19). In some embodiments, provided herein are recombinant virions comprising at least one capsid protein of an erythroparvovirus and a nucleic acid, wherein the nucleic acid comprises a heterologous nucleic acid, and the erythroparvovirus is not human erythroparvovirus B19.

In some embodiments, a heterologous nucleic acid comprises a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a nucleic acid sequence of a target cell. In some embodiments, a heterologous nucleic acid comprises a nucleic acid sequence that is at least about 80% identical to a nucleic acid sequence of a target cell.

In some embodiments, a recombinant virion comprises a heterologous nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a nucleic acid of a mammal, preferably wherein the mammal is a human.

In some embodiments, a recombinant virion comprises a heterologous nucleic acid that is not operably linked to a human erythroparvovirus B19 promoter. A human erythroparvovirus B19 promoter has not shown effective regulation of a heterologous nucleic acid in a target cell (e.g., mammalian cell).

In some embodiments, a nucleic acid comprises at least one inverted terminal repeat (ITR). In some embodiments, a nucleic acid comprises two ITRs. ITR may comprise a dependoparvovirus ITR. In some such embodiments, the at least one ITR may comprise an AAV ITR. In some embodiments, the AAV ITR is an AAV2 ITR. In some embodiments, the at least one ITR may comprise an erythroparvovirus ITR. In some embodiments, an ITR is an ITR of the human erythroparvovirus B19 or a genotypic variant thereof.

A recombinant virion may be icosahedral. In preferred embodiments, a recombinant virion may comprise at least one capsid protein of human erythroparvovirus B19 or a genotypic variant thereof. In some embodiments, a recombinant virion may comprise at least one capsid protein of any one of virions selected from: primate erythroparvovirus 4 (pig-tailed macaque parvovirus), primate erythroparvovirus 3 (rhesus macaque parvovirus), primate erythroparvovirus 2 (simian parvovirus), rodent erythroparvovirus 1, ungulate erythroparvovirus 1, or a genotypic variant thereof.

A capsid protein may comprise at least one structural protein such as a VP1 capsid protein. A capsid protein may comprise at least one structural protein such as a VP2 capsid protein. A capsid protein may comprise a VP1 capsid protein and a VP2 capsid protein. In some embodiments, a VP1 capsid protein comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NO: 9. In some embodiments, a VP2 capsid protein comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NO: 11. In some embodiments, a capsid protein comprises a VP1 capsid protein and a VP2 capsid protein. VP2 may be present in excess of VP1. For example, VP2 may be present in excess of VP1 by at least about 1%, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 9000%, or 10000%).

In some embodiments, a VP1 capsid protein is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 6, SEQ ID NO: 7, or SEQ ID NO: 8. In some embodiments, a VP1 capsid protein is encoded by a nucleic acid that is codon-optimized for expression. In some embodiments, a VP2 capsid protein is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 10. In some embodiments, a VP2 capsid protein is encoded by a nucleic acid that is codon-optimized for expression.

In some embodiments, a nucleic acid of a recombinant virion is deoxyribonucleic acid (DNA). DNA may be single-stranded or self-complementary duplex. In some embodiments, a nucleic acid may comprise a Rep protein-dependent origin of replication (ori), thereby allowing replication of said nucleic acid (e.g., for vector production).

In certain embodiments, a nucleic acid comprises a nucleic acid operably linked to a promoter, optionally placed between two ITRs. A nucleic acid operably linked to a promoter may comprise a heterologous nucleic acid encoding a coding RNA. In some embodiments, a coding RNA comprises (a) a gene encoding a protein or a fragment thereof, preferably a human protein or a fragment thereof; (b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof); (c) a nucleic acid encoding a reporter, e.g., luciferase or GFP; or (d) a nucleic acid encoding a drug resistance protein, e.g., neomycin resistance. In some embodiments, a heterologous nucleic acid encoding a coding RNA is codon-optimized for expression in a target cell. In some embodiments, a heterologous nucleic acid operably linked to a promoter comprises a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, cystic fibrosis transmembrane conductance regulator (CFTR), F8 or a fragment thereof (e.g., fragment encoding B-domain deleted polypeptide (e.g., VIII SQ, p-VIII)), Lysosomal storage diseases, and/or any of the genes disclosed herein. In certain embodiments, a nucleic acid operably linked to a promoter may comprise a heterologous nucleic acid encoding a non-coding RNA. In some embodiments, a non-coding RNA comprises lncRNA, miRNA, shRNA, siRNA, antisense RNA, and/or gRNA.

In certain embodiments, a nucleic acid operably linked to a promoter may encode a coding RNA, a protein, or a non-coding RNA that increases or restores the expression of an endogenous gene of a target cell. In some embodiments, a nucleic acid operably linked to a promoter may encode a coding RNA, a protein, or a non-coding RNA that decreases or eliminates the expression of an endogenous gene of a target cell.

In certain embodiments, a nucleic acid is operably linked to a promoter selected from: (a) a promoter heterologous to a nucleic acid, (b) a promoter that facilitates the tissue-specific expression of a nucleic acid, preferably wherein the promoter facilitates hematopoietic cell-specific expression or erythroid lineage-specific expression, (c) a promoter that facilitates the constitutive expression of a nucleic acid, and (d) a promoter that is inducibly expressed, optionally in response to a metabolite or small molecule or chemical entity. In some embodiments, a promoter is a human erythroparvovirus B19 promoter. In some embodiments, a promoter is not a human erythroparvovirus B19 promoter. In some embodiments, a promoter is selected from the CMV promoter, β-globin promoter, CAG promoter, AHSP promoter, MND promoter, Wiskott-Aldrich promoter, and PKLR promoter. In some embodiments, a nucleic acid is not operably linked to a promoter in the vectors, and is instead dependent on homology-dependent repair (HDR) for incorporation into a genomic region for expression, either into a heterologous locus—for example, utilizing HDR into an albumin exon to produce a fusion protein, or into the homologous genetic locus to restore the open reading frame. In either of these cases, the vector DNA remains “silent” unless integrated into the cellular genome at a site that enables transcriptional activity.

In certain embodiments, a nucleic acid comprises a non-coding DNA. In some embodiments, a non-coding DNA comprises a transcription regulatory element (e.g., an enhancer, a transcription termination sequence, an untranslated region (5′ or 3′ UTR), a proximal promoter element, a locus control region, or a polyadenylation signal sequence). In some such embodiments, a transcription regulatory element may be a locus control region, optionally a β-globin LCR or a DNase hypersensitive site (HS) of β-globin LCR. In some embodiments, the non-coding DNA comprises a translation regulatory element (e.g., Kozak sequence, woodchuck hepatitis virus post-transcriptional regulatory element).

In some embodiments, a recombinant virion comprises a nucleic acid sequence encoding replication proteins and/or at least one capsid protein. In some embodiments, a recombinant virion is autonomously replicating.

In certain embodiments, a recombinant virion described herein binds and/or transduces a hematopoietic cell and/or a cell expressing erythrocyte P antigen. In some embodiments, a recombinant virion binds and/or transduces (a) an erythroid lineage cell, (b) a cancerous erythroid lineage cell, (c) a hematopoietic stem cell (HSC), or (d) a cell expressing CD36 and/or CD34. In some embodiments, an erythroid lineage cell is a megakaryocyte or an erythroid progenitor cell (EPC), optionally a CD36+ EPC. In certain embodiments, a recombinant virion binds and/or transduces a non-erythroid linage cell or a cancerous non-erythroid lineage cell. In some embodiments, a non-erythroid lineage cell is an endothelial cell, optionally a myocardial endothelial cell. In some embodiments, a non-erythroid lineage cell is a hepatocyte. In preferred embodiments, a virion transduces a cell in an erythrocyte P antigen-dependent manner.

### Pharmaceutical Compositions

In some embodiments, provided herein are pharmaceutical compositions comprising a recombinant virion described herein and a carrier and/or a diluent. As used herein the pharmaceutically acceptable carrier is intended to include any and all solvents, dispersion media, antibacterial and antifungal agents, isotonic and absorption delaying agents, and the like, compatible with pharmaceutical administration. Use of such media and agents for pharmaceutically active substances is well-known in the art. Except insofar as any conventional media or agent is incompatible with the active compound, use thereof in the compositions is contemplated. For determining compatibility, various relevant factors, such as osmolarity, viscosity, and/or baricity can be considered. Supplementary active compounds can also be incorporated into pharmaceutical compositions.

A pharmaceutical composition of the present invention is formulated to be compatible with its intended route of administration. Examples of routes of administration include parenteral, e.g., intravenous, intradermal, subcutaneous, oral, intranasal (e.g., inhalation), transdermal, transmucosal, and rectal administration. In certain embodiments, a direct injection into the bone marrow is contemplated. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For example, Ringer's solution and lactated Ringer's solution are USP approved for formulating IV therapeutics, and those solutions are used in some embodiments. In certain embodiments, the excipient and vector compatibility to retain biological activity is established according to suitable methods. For intravenous administration or injection to the bone marrow, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like, to the extent that they do not affect the integrity/activity of the viral compositions described herein. In many cases, it is preferable to include isotonic agents, for example, sugars, polyalcohols such as manitol, sorbitol, sodium chloride in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

For administration by inhalation, viral particles described herein are delivered in the form of an aerosol spray from pressured container or dispenser which contains a suitable propellant, e.g., a gas such as carbon dioxide, or a nebulizer.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through use of nasal sprays or suppositories.

### Erythroparvovirus

Members of this genus are primarily distinguished by sequence identity criteria. Genomes are homotelomeric, ˜5.5 kb, and are bracketed by terminal repeats (TRs) that end in long (˜365 nt) palindromic telomeres. Several erythroparvoviruses preferentially target human erythroid progenitor cells.

The N-terminal VP1 of an erythroparvovirus differs from those encoded by most parvoviruses in being unusually long (227 amino acids) and by being positioned on the outside of infectious virions before entering cells. It includes a PLA2 domain, which is involved in endosomal escape. X-ray crystallographic structures of VP2-only erythroparvovirus-like particles (VLPs) and cryo-EM image reconstructions of DNA-containing erythroparvovirus virions and empty particles from human sera show that a conserved glycine-rich VP peptide, which has been observed within the channel in virions from some other genera, lies between neighboring VP chains at the five-fold axis of symmetry that forms a pore that extends from outer to inner surfaces of the capsid that accommodates virus DNA packaging, and effectively position most of the extreme VP2 N-termini on the particle surface next to the cylinder of the trans-capsid pore. These models also indicate that the erythroparvovirus fivefold channel itself is relatively short and appears constricted at an outer viral surface, gated by five symmetry-related threonines. However, without wishing to be bound to any theory, it is hypothesized that three glycine residues (amino acids 136-138) immediately N-terminal to threonines could provide structural flexibility required for switching a channel from closed to open during virion maturation. Overall, these studies indicate that in erythroparvovirus the pore at a fivefold axis mediates transit of a single-stranded DNA into a capsid.

In some erythroparvoviruses, the homotelomeric genome is 5,596 nt, with long (383 nt) terminal repeats (TRs) that end in imperfectly palindromic hairpins of 365 nt. The integrity of hairpins contributes to viral infectivity, although why they are so complex remains unclear. However, it is known that the signal transducer and activator of transcription 5 (STAT5), which plays an important role in viral DNA replication, specifically interacts with TRs (Ganaie et al., 2017). The genome has a single transcriptional promoter (P6), which gives rise to one full-length pre-mRNA, and two polyadenylation signals, one corresponding to the middle of the DNA (p(A)p) and the other (p(A)d) near its right end. A single pre-mRNA is alternatively spliced at one or two introns using a total of two donor and four acceptor sites, generating 12 viral mRNAs that encode the replication initiator protein (a nonstructural protein (e.g., NS, NS1, and/or NS2)), two structural proteins (e.g., VP1 capsid protein and VP2 capsid protein) and two ancillary proteins (7.5 kDa and 11 kDa). During the early infection phase DNA replication amplifies a virus genome. The transition from early to late infection phase is marked by the transcriptional read-through of the pAp signal and utilization of the distal pAd signal resulting in expression of structural proteins VP1 capsid protein and VP2 capsid protein. An intronic splice enhancer (ISF2) that contains a binding site for a cellular RNA binding protein (RBM38) lies immediately distal to the D2 donor. RBM38 expression during erythropoiesis makes it available to bind to ISE2, leading to enhanced recognition of the D2 splice site and high-level expression of the 11 kDa protein (Ganaie et al., 2018). The temporally regulated 11 kDa ancillary protein is known to be a potent inducer of apoptosis in erythroid progenitor cells (Chen et al., 2010b) and is essential for optimal viral DNA replication and virion release (Ganaie et al., 2018), whereas the function of the 7.5 kDa protein remains uncertain. Apoptosis is a cellular antiviral response that kills a cell prior to replication and therefore, lytic viruses may encode apoptosis inhibitors.

Some erythroparvoviruses have narrow tissue tropism that in culture restricts its productive replication to a short time period following differentiation of human bone marrow CD34+ stem cells into CD36+ erythroid progenitor cells (EPCs) (reviewed in detail in (Qiu et al., 2017)). It can also replicate productively, albeit much less efficiently, in a human megakaryoblastoid cell line, UT7/Epo-S1. The viability of both of these productive cell types depends upon access to erythropoietin (Epo), and Epo/Epo-receptor (Epo-R) signaling plays a critical role in promoting infection via activation of Janus kinase 2 (Jak2) pathways. Jak2 further expands Epo-R phosphorylation and initiates a kinase cascade that activates STAT5A transcription and down-regulates signaling by mitogen-activated protein kinase (MEK/ERK), both of which lead to enhanced virus production. Culturing cells under hypoxic conditions (1% O₂) to mimic the environment in human bone marrow, also significantly increases viral DNA replication and progeny virus production (Pillet et al., 2004), although in EPCs this acts by regulating EpoR signaling rather than by a more common HIF-1α pathway (Luo and Qiu 2015). Viral infection of EPCs also induces a DNA damage response (DDR) with activation of all three phosphatidylinositol 3-kinase-related kinases (PI3KKs). The virus hijacks the induced ATR and the DNA-PKcs pathways to promote viral DNA amplification, inducing cell cycle arrest in late S phase that allows DNA replication resources of a cell to be diverted for replication of viral DNA (Luo and Qiu 2015, Zou et al., 2018).

In children, erythroparvovirus infection of EPCs commonly manifests as an immune complex exanthema called “fifth” disease, also known as erythema infectiosum or “slapped-cheek” syndrome, while in adults (especially women) polyarthralgia is common. In vulnerable populations a range of additional clinical disorders may occur. For example, EPC disfunction can cause persistent anemia in immunosuppressed individuals, transient aplastic crisis in patients who require increased erythropoiesis (e.g. in sickle cell disease), or chronic pure red cell aplasia in congenitally immune-compromised patients. A virus can also cross a placenta, sometimes resulting in hydrops fetalis in developing 2nd trimester fetuses. Clinical observations suggest that erythroparvovirus could also be implicated in hepatic or cardiovascular diseases such as myocarditis, certain autoimmune conditions and chronic fatigue syndrome, possibly by being taken into and perturbing non-productive cell types in these conditions, although how a virus induces such pathology requires further study (Qiu et al., 2017, Luo and Qiu 2015, Kerr 2016).

Many erythroparvoviruses, e.g., those that infect simian, pig-tailed or rhesus macaques all show a predilection for the bone marrow and can induce significant anemia in immunosuppressed animals (Brown and Young 1997, Green et al., 2000), suggesting common cell type specificities.

TABLE 1
Exemplar Isolate of the Species of Erythroparvovirus

| | Virus | Accession | Ref |
|---|---|---|---|
| Species | Name | No. | Seq No. |
| Primate erythroparvovirus 1 | Human | AY386330 | NC_000883 |
| | erythroparvovirus B19 | | |
| Primate erythroparvovirus 2 | Simian parvovirus | U26342 | NC_038540 |
| Primate erythroparvovirus 3 | Rhesus macaque | AF221122 | NC_038541 |
| | parvovirus | | |
| Primate erythroparvovirus 4 | Pig-tailed macaque | AF221123 | NC_038542 |
| | parvovirus | | |
| Rodent erythroparvovirus 1 | Chipmunk parvovirus | GQ200736 | NC_038543 |
| Ungulate erythroparvovirus 1 | Bovine parvovirus 3 | AF406967 | NC_037053 |

### Genotypic Variants

A person of ordinary skill in the art would understand that there are genotypic variants of viruses. For example, a certain erythroparvovirus coding sequences were first cloned and sequenced in the early 1980s (Cotmore and Tattersall 1984, Shade et al., 1986) and for many years the same highly-conserved genotype (now called G1) was observed in western populations, but by 2002 two relatively rare variants had been reported, now called G2 and G3, which diverge in genome nucleotide sequence by ˜10% (Nguyen et al., 1999, Hokynar et al., 2002, Nguyen et al., 2002, Servant et al., 2002). Previously it had been observed that following primary viral infections, viral genomes commonly persist in solid tissues (Söderlund et al., 1997, Söderlund-Venermo et al., 2002, Hokynar et al., 2007), at least in part due to antibody mediated virus internalization by B-lymphocytes (Pyöriä et al., 2017). However, although G1 remains a predominant virus in circulation globally, both G1 and G2 forms could be found in solid tissue samples (in 25% and 11% of samples, respectively), with G1 occurring in tissues from all age groups whereas G2 was strictly confined to the tissues of subjects born before 1973 (Norja et al., 2006). This suggested that G2 had been in circulation until the early 1970s, but had since been replaced by G1. Genomes retained in solid tissues were therefore dubbed an erythrovirus “bioportfolio”, since they provide a permanent record of the viruses responsible for each individual's infectious history (Norja et al., 2006). In subsequent studies genotypes were assessed in the skeletal remains of World War II battle casualties from Finland, and found to be exclusively G2 (n=41) or G3 (n=2), indicating that G1 was likely absent in this area during the first half of the 20th century, while G2 was the major circulating virus. G3 appears to be a geographic variant that had previously been seen only in Ghana, Brazil and India, and both of the G3 tissue samples mentioned above were associated with human genotypic markers suggestive of non-European origins, likely reflecting the wider cultural diversity of Soviet armies (Toppinen et al., 2015). Where or when G1 arose and why it became pre-eminent remains uncertain, but to date there are no biological differences between viruses from the three genotypes and they all belong to the same serotype (Blümel et al., 2005, Ekman et al., 2007, Chen et al., 2009).

Accordingly, the term “erythroparvovirus” includes the genetic variants thereof.

### Genomic Safe Harbors (GSHs)

Genomic safe harbors (GSH) are intragenic, intergenic, or extragenic regions of the human and model species genomes that are able to accommodate predictable expression of newly integrated DNA without significant adverse effects on a host cell or organism. GSHs may comprise intronic or exonic gene sequences as well as intergenic or extragenic sequences. While not being limited to theory, a useful safe harbor must permit sufficient transgene expression to yield desired levels of a transgene-encoded protein or non-coding RNA. A GSH also should not predispose cells to malignant transformation, nor interfere with progenitor cell differentiation, nor significantly alter normal cellular functions. What distinguishes a GSH from a fortuitous good integration event is predictability of outcome, which is based on prior knowledge and validation of a GSH.

A larger genome size of a recombinant virion described herein allows delivery of a therapeutic transgene(s) together with GSH sequences, which is otherwise not possible with virions having a limited genome size, e.g., AAV. Accordingly, recombinant virions of the present disclosure not only facilitates delivery of a larger transgene compared with e.g., AAV, but also facilitates a safe delivery of a transgene by allowing codelivery of a GSH sequences that ensures predictable expression of the transgene without adverse effects on host cells.

Exemplary GSHs that have been targeted for transgene addition include (i) the adeno-associated virus site 1 (AAVS1), a naturally occurring, non-germline, site of integration of AAV virus DNA on chromosome 19; (ii) the chemokine (C-C motif) receptor 5 (CCR5) gene, a chemokine receptor gene known as an HIV-1 coreceptor; (iii) the human ortholog of the mouse Rosa26 locus, a locus extensively validated in the murine setting for the insertion of ubiquitously expressed transgenes; and (iv) albumin in murine cells (see, e.g., U.S. Pat. Nos. 7,951,925; 8,771,985; 8,110,379; and 7,951,925; U.S. Patent Publication Nos. 2010/0218264; 2011/0265198; 2013/0137104; 2013/0122591; 2013/0177983; 2013/0177960; 2015/0056705 and 2015/0159172; all of which are incorporated by reference). Additional GSHs include Kif6, Pax5, collagen, HTRP, HI 1 (a thymidine kinase encoding nucleic acid at HI 1 locus), beta-2 microglobulin, GAPDH, TCR, RUNX1, KLHL7, NUPL2 or an intergenic region thereof, mir684, KCNH2, GPNMB, MIR4540, MIR4475, MIR4476, PRL32P21, LOC105376031, LOC105376032, LOC105376030, MELK, EBLN3P, ZCCHC7, RNF38, or loci meeting the criteria of a genome safe harbor as described herein (see e.g., WO 2019/169233 A1, WO 2017/079673 A1; incorporated by reference). These GSHs provide a non-limiting representation of GSHs that can be used with recombinant virions described herein. The present disclosure contemplates use of any GSHs that are known in the art.

In some embodiments, GSH allows safe and targeted gene delivery that has limited off-target activity and minimal risk of genotoxicity, or causing insertional oncogenesis upon integration of foreign DNA, while being accessible to highly specific nucleases with minimal off-target activity.

In some embodiments, GSH has any one or more of the following properties: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral vector integrations in transduced induced pluripotent stem cells, analysis of over 5,000 integration sites revealed that ˜17% of integrations occurred in safe harbors. The vectors that integrated into these safe harbors were able to express therapeutic levels of β-globin from their transgene without perturbing endogenous gene expression.

In some embodiments, GSH is AAVS1. AAVS1 was identified as the adeno-associated virus common integration site on chromosome 19 and is located in chromosome 19 (position 19q13.42) and was primarily identified as a repeatedly recovered site of integration of wild-type AAV in the genome of cultured human cell lines that have been infected with AAV in vitro. Integration in the AAVS1 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAVS1. Originally, AAV DNA integration into AAVS1 site was Rep-dependent, however, there are commercially available CRISPR/Cas9 reagents available for targeting which preserved the functionality of the targeted allele and maintained the expression of PPP1R12C at levels that are comparable to those in non-targeted cells. AAVS1 was also assessed using ZFN-mediated recombination into iPSCs or CD34+ cells.

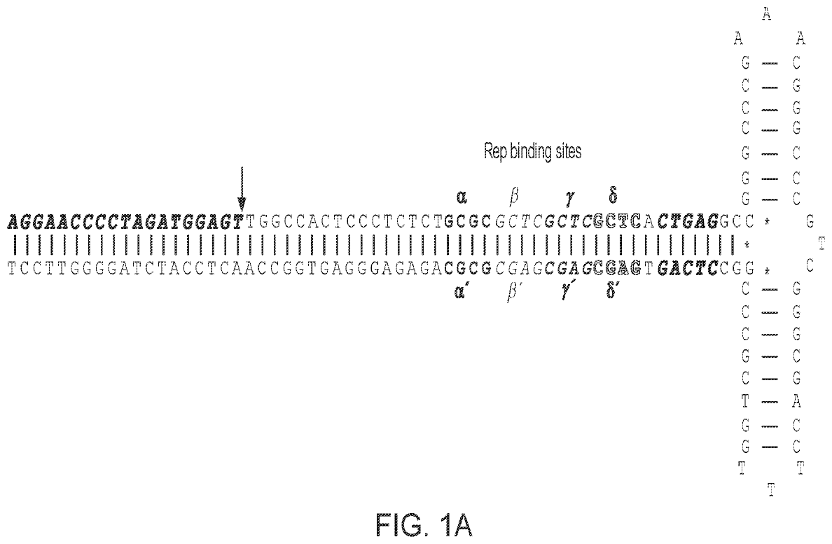

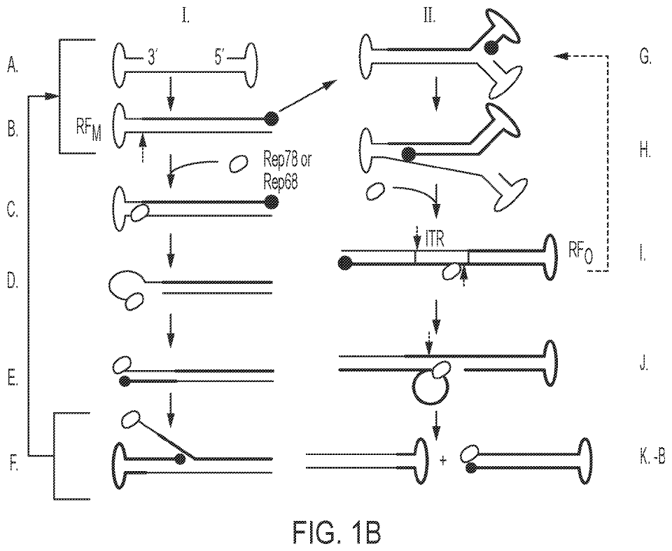

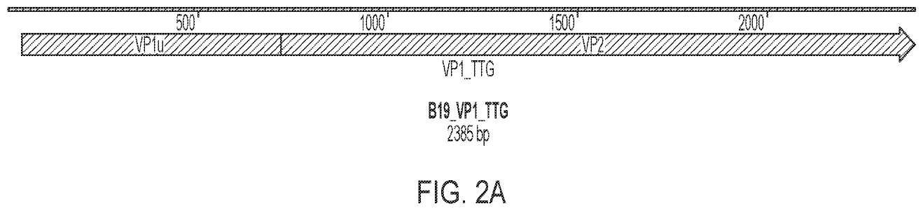

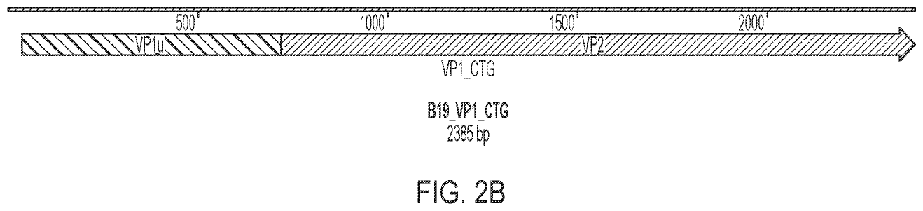

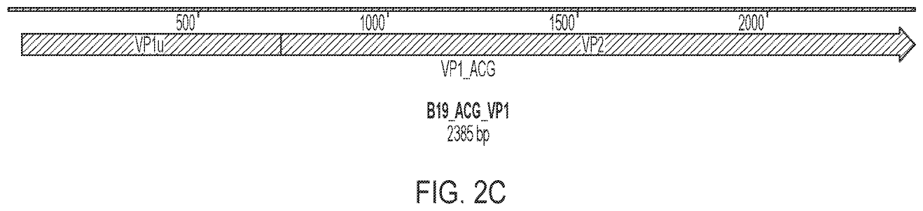

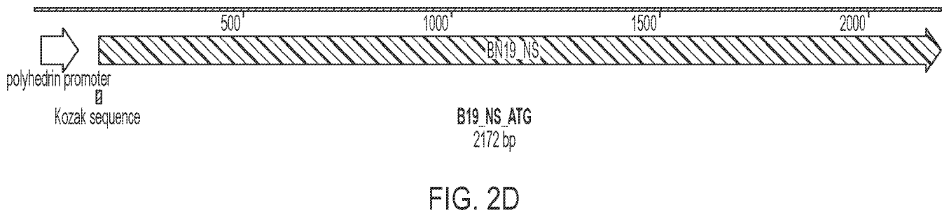

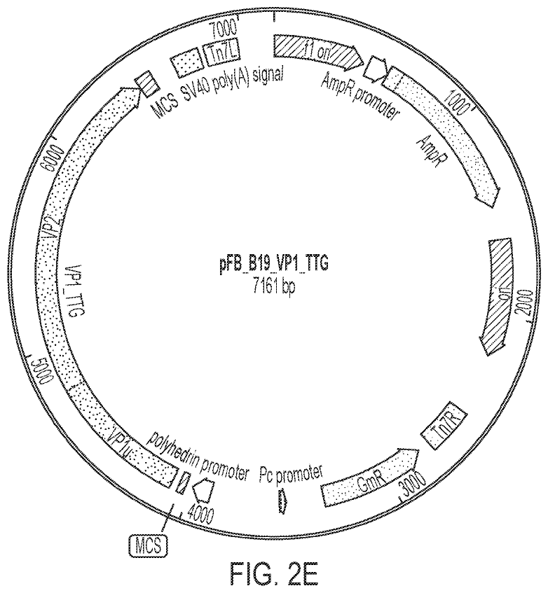

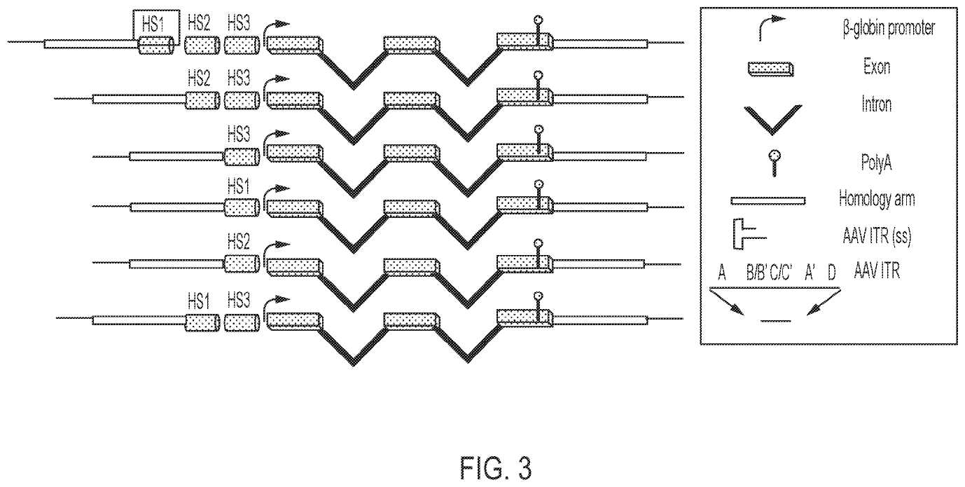

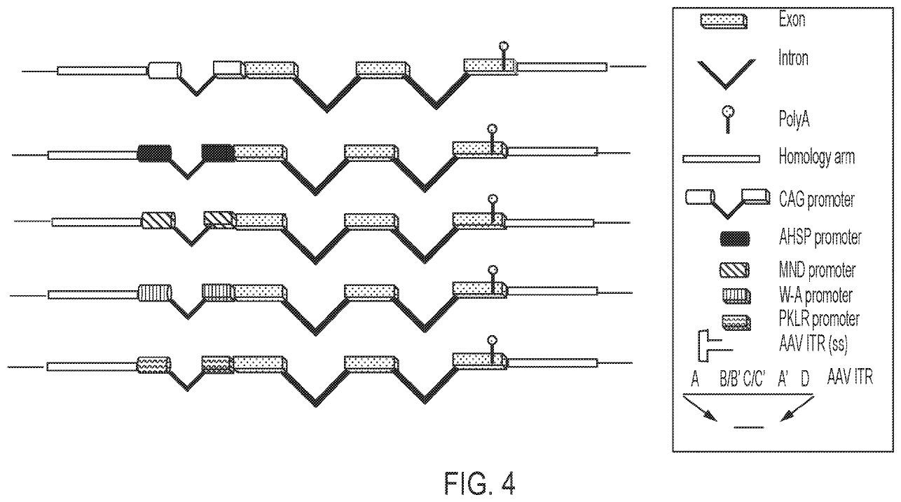

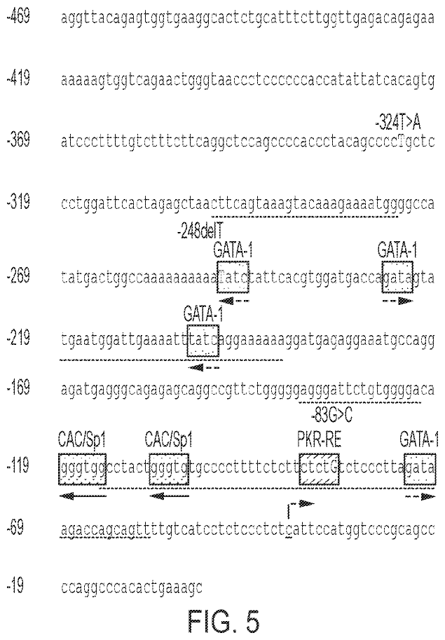

As originally characterized, the AAVS1 locus is >4 kb and is identified as chromosome 19 nucleotides 55,1 13,873-55,1 17,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is a gene-rich region of particularly gene-rich chromosome 19 (see FIG. 1A of Sadelain et al, Nature Revs Cancer, 2012; 12; 51-58), and some integrated promoters can indeed activate or cis-activate neighboring genes, the consequence of which in different tissues is presently unknown. PPP1R12C exon 1 5′untranslated region contains a functional AAV origin of DNA synthesis indicated within a known sequence (Urcelay et al. 1995).

AAVS1 GSH was identified by characterizing AAV provirus structure in latently infected human cell lines with recombinant bacteriophage genomic libraries generated from latently infected clonal cell lines (Detroit 6 clone 7374 IIID5) (Kotin and Berns 1989), Kotin et al, isolated non-viral, cellular DNA flanking a provirus and used a subset of “left” and “right” flanking DNA fragments as probes to screen panels of independently derived latently infected clonal cell lines. In approximately 70% of the clonal isolates, AAV DNA was detected with a cell-specific probe (Kotin et al. 1991; Kotin et al. 1990). Sequence analysis of a pre-integration site identified near homology to a portion of the AAV inverted terminal repeat (Kotin, Linden, and Beerns 1992). Although lacking a characteristic interrupted palindrome, the AAVS1 locus retained the Rep binding elements and terminal resolution sites homologous to an AAV ITR (FIG. 1A).

Selection of an exonic integration site is non-obvious, and perhaps counter-intuitive, since insertion and expression of foreign DNA likely disrupts expression of endogenous genes. Apparently, insertion of an AAV genome into this locus does not adversely affect cell viability or iPSC differentiation (DeKelver et al. 2010; Wang et al. 2012; Zou et al. 2011). The AAVS1 locus is within the 5′ UTR of the highly conserved PPP1R12C gene. The Rep-dependent minimal origin of DNA synthesis is conserved in the 5′UTR of the human, chimpanzee, and gorilla PPP1R12C gene. However, the commercially available CRISPR/Cas9 reagents used for integrating DNA into AAVS1 target PPP1R12C intron 1 rather than the exon.

In some embodiments, GSH is any one of Kif6, Pax5, collagen, HTRP, HI 1, beta-2 microglobulin, GAPDH, TCR, RUNX1, KLHL7, an intergenic region of NUPL2, mir684, KCNH2, GPNMB, MIR4540, MIR4475, MIR4476, PRL32P21, LOC105376031, LOC105376032, LOC105376030, MELK, EBLN3P, ZCCHC7, and RNF38.

In some embodiments, GSH is the Pax5 gene (also known as Paired Box 5, or “B-cell lineage specific activator protein,” or BSAP). In humans PAX5 is located on chromosome 9 at 9p 13.2 and has orthologues across many vertebrate species, including, human, chimp, macaque, mouse, rat, dog, horse, cow, pig, opossum, platypus, chicken, lizard, Xenopus, C. elegans, Drosophila and zebrafish. PAX5 gene is located at Chromosome 9:36, 833,275-37,034,185 reverse strand (GRCh38: CM000671.2) or 36,833,272-37,034,182 in GRCh37 coordinates.

Additional exemplary GSHs are listed in Table 2A and Table 2B.

TABLE 2A
Exemplary GSH loci in Homo Sapiens (see, e.g.,
WO 2019/169232; incorporated by reference)

| Gene | Chromosomal location | Accession number/location |
|---|---|---|
| PAX5 | Chromosome 9: 36,833,275- | NC_000009.12 (36833274 . . . 37035949, |
| | 37,034,185 reverse strand | complement) |
| MIR4540 | | NC_000009.12 (36864254 . . . 36864308, |
| | | complement) |
| MIR4475 | GRCh38.p7 | NC_000009.12 (36823539 . . . 36823599, |
| | (GCF_000001405.33) | complement) |
| MIR4476 | GRCh38.p7 | NC_000009.12 (36893462 . . . 36893531, |
| | (GCF_000001405.33) | complement) |
| PRL32P21 | GRCh38.p7 | NC_000009.12 (37046835 . . . 37047242) |
| | (GCF_000001405.33) | |
| LOC105376031 | GRCh38.p7 | NC_000009.12 (37027763 . . . 37031333) |
| | (GCF_000001405.33) | |
| LOC105376032 | GRCh38.p7 | NC_000009.12 (37002697 . . . 37007774) |
| | (GCF_000001405.33) | |
| LOC105376030 | GRCh38.p7 | NC_000009.12 (36779475 . . . 36830456) |
| | (GCF_000001405.33) | |
| MELK | GRCh38.p7 | NC_000009.12 (36572862 . . . 36677683) |
| | (GCF_000001405.33) | |
| EBLN3P | GRCh38.p7 | NC_000009.12 (37079896 . . . 37090401) |
| | (GCF_000001405.33) | |
| ZCCHC7 | GRCh38.p7 | NC_000009.12 (37120169 . . . 37358149) |
| | (GCF_000001405.33) | |
| RNF38 | GRCh38.p7 | NC_000009.12 (36336398 . . . 36487384, |
| | (GCF_000001405.33) | complement) |

TABLE 2B
Exemplary GSH loci (see, e.g., WO 2019/169232; incorporated by reference)

| | | | |
|---|---|---|---|
| Taxonomic | Brief | | Chromoson [symbol] al |
| Rank | description | Species | location |

[symbol]

ntergenic loci

| | | | |
|---|---|---|---|
| Macropodidae | - mAAV_eve integration between | M. domestica | chromosome 1: |
| (taxonomic | cadherin (cdh) 8 and cdh 16. | | cdh 8: 674,639,xxx-675,163,xxx |
| rank: Family) | Because the macropod genome is | | cdh 10: 680,370,7xx-680,581,xxx |
| | poorly annotated, another | | [symbol] ntergenic distance = 5.2 Mb |
| | marsupial Mondelphis domesitca | | Empty EVE locus in M. domestica 674,422,470-675,422,729 |
| | with a more completely assemble | Mouse | ch 9: |
| | genome is used as a substitute | | cdh 8: 99,028,769-99,416,471 |
| | genome. | | cdh 11: 192,632,095-102,785,111 |
| | | | [symbol] ntergenic distance = 3.2 Mb |
| | | Homo sapiens | Chromesome 16 |
| | | | cdh 8: 61,647,242-62,036,835 |
| | | | cdh 11: 64, [symbol] 43,753-65,122,1 [symbol] |
| | | | Intergenic distance = 2.9 Mb |
| Leporidae | Leporidae EVE located between | H. Sapiens | Chromosome 7: |
| (Family) - the | NupL2 and GPNMB | | -KH7->-NUPLZ→GPNMB |
| Family | The gang order is: | M. mus | -KLHL7->-NUPL2→ mi [symbol] 84-KCNH2 |
| Leporidae are | <-Fam126A- -KLM7-> [symbol] NUPL2-> [symbol] | | |
| rabbits and | -EVE [symbol] GPNM8-> [symbol] <IGF28P3 [symbol] | | |
| hares species | MALSU1 | | |
| of the | | | |
| Lagomorph | | | |
| Order. | | | |

[symbol]

ntragenic loci

| | | | |
|---|---|---|---|
| Cetacea | EVE integrated into an in [symbol] tron | H. sapiens | chromosome 9: |
| (Order) | of PAX5 | | (Pax5) 36,833,275-37,034,185 |
| | | M. mus | Chromosome 4: |
| | | | (Pax5) 44,531,506-44,710,440 |
| (Family - | Myotis EVE integrated into the Kif6 | H. sapiens | Chromosome 6 |
| Vespertilionidae, | gene, intronic or exonic | | (K [symbol] 5} 39,329,990-39,725,405 |
| Order - | | M. mus | Chromosome 17 |
| Chiroptera). | | | (K [symbol] 6} 49,754,497-50,049,172 |
| Myotis | | | |
| (Genus), | | | |
| Myo [symbol] ae | | | |
| (Subfamily) | | | |

[symbol]

indicates data missing or illegible when filed

### Integration to a Target Genome

Integration to a target genome may be driven by cellular processes, such as homologous recombination or non-homologous end-joining (NHEJ). Integration may also be initiated and/or facilitated by an exogenously introduced nuclease. In preferred embodiments, a nucleic acid packaged within recombinant virions described herein is integrated to a specific locus within the genome, e.g., GSH. In some embodiments, a GSH is any locus that permits sufficient transgene expression to yield desired levels of a transgene-encoded protein or non-coding RNA. A GSH also should not predispose cells to malignant transformation nor significantly alter normal cellular functions. Site-specific integration to a GSH may be mediated by a nucleic acid homologous to a GSH that is placed 5′ and 3′ to a nucleic acid to be integrated. Such homologous donor sequences may provide a template for homology-dependent repair that allows integration at a desired locus.

In preferred embodiments, a recombinant virion described herein comprises a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a nucleic acid sequence of a genomic safe harbor (GSH) of the target cell. In some embodiments, the said nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a GSH is placed 5′ and 3′ (homology arms) to a nucleic acid to be integrated, thereby allowing insertion (of a nucleic acid located between the homology arms) to a specific locus in the target genome by homologous recombination. In some embodiments, a nucleic acid to be integrated is a nucleic acid operably linked to a promoter described herein. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2.

In certain embodiments, a nucleic acid of a recombinant virion is integrated into the genome of a target cell upon transduction. In some embodiments, a nucleic acid is integrated into a GSH or EVE. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2. In some embodiments, a nucleic acid is integrated into the target genome by homologous recombination followed by a DNA break formation induced by an exogenously-introduced nuclease. In some embodiments, a nuclease is TALEN, ZFN, a meganuclease, a megaTAL, or a CRISPR endonuclease (e.g., a Cas9 endonuclease or a variant thereof). In some embodiments, a CRISPR endonuclease is in a complex with a guide RNA.

In some embodiments, provided herein are methods of integrating a heterologous nucleic acid into a GSH in a cell, comprising: (a) transducing a cell with one or more virions described herein comprising a heterologous nucleic acid flanked at the 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a target GSH nucleic acid; or (b) transducing a cell with one or more virions described herein comprising (i) a heterologous nucleic acid flanked at the 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a target GSH nucleic acid, and (ii) a nucleic acid encoding a nuclease (e.g., Cas9 or a variant thereof, ZFN, TALEN) and/or a guide RNA, wherein a nuclease or a nuclease/gRNA complex makes a DNA break at a GSH, which is repaired using a donor nucleic acid, thereby integrating a heterologous nucleic acid at GSH. In some embodiments, (i) a heterologous nucleic acid flanked by a donor nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a target GSH nucleic acid and (ii) a nucleic acid encoding a nuclease and/or the gRNA are transduced in separate virions. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2.

For integration of a nucleic acid located between the 5′ and 3′ homology arms, the 5′ and 3′ homology arms should be long enough for targeting to a GSH and allow (e.g., guide) integration into the genome by homologous recombination. To increase the likelihood of integration at a precise location and enhance the probability of homologous recombination, the 5′ and 3′ homology arms may include a sufficient number of nucleic acids. In some embodiments, the 5′ and 3′ homology arms may include at least 10 base pairs but no more than 5,000 base pairs, at least 50 base pairs but no more than 5,000 base pairs, at least 100 base pairs but no more than 5,000 base pairs, at least 200 base pairs but no more than 5,000 base pairs, at least 250 base pairs but no more than 5,000 base pairs, or at least 300 base pairs but no more than 5,000 base pairs. In some embodiments, the 5′ and 3′ homology arms include about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 base pairs. Detailed information regarding the length of homology arms and recombination frequency is art-known, see e.g., Zhang et al. “Efficient precise knock in with a double cut HDR donor after CRISPR/Cas9-mediated double-stranded DNA cleavage.” Genome biology 18.1 (2017): 35, which is incorporated herein in its entirety by reference.

The 5′ and 3′ homology arms may be any sequence that is homologous with a GSH target sequence in the genome of a host cell. In some embodiments, the 5′ and 3′ homology arms may be homologous to portions of a GSH described herein. Furthermore, the 5′ and 3′ homology arms may be non-coding or coding nucleotide sequences.

In some embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 75, 100, 125, 150, 175, 200, 225, 250, 275, 300, 325, 350, 375, 400, 425, 450, 475, 500 or more base pairs away from the integration or DNA cleavage site, or partially or completely overlapping with the DNA cleavage site (e.g., can be a DNA break induced by an exogenously-introduced nuclease). In some embodiments, the 3′ homology arm of the nucleotide sequence is proximal to an ITR.

### Gene-Editing Systems

In some embodiments, the methods and compositions described herein are used to integrate a nucleic acid delivered by a recombinant virion described herein into any specific locus (e.g., GSH) within a target genome. In some embodiments, integration is initiated and/or facilitated by an exogenously introduced nuclease, and the DNA break induced by a nuclease is repaired using the homology arms as a guide for homologous recombination, thereby inserting a nucleic acid flanked by the said homology arms into a target genome.

For example, a double-strand break (DSB) for can be created by a site-specific nuclease such as a zinc-finger nuclease (ZFN) or TAL effector domain nuclease (TALEN). See, for example, Urnov et al. (2010) Nature 435 (7042): 646-51; U.S. Pat. Nos. 8,586,526; 6,534,261; 6,599,692; 6,503,717; 6,689,558; 7,067,317; 7,262,054, the disclosures of which are incorporated by reference.

Another nuclease system involves the use of a so-called acquired immunity system found in bacteria and archaea known as the CRISPR/Cas system. CRISPR/Cas systems are found in 40% of bacteria and 90% of archaea and differ in the complexities of their systems. See, e.g., U.S. Pat. No. 8,697,359. The CRISPR loci (clustered regularly interspaced short palindromic repeat) are regions within an organism's genome where short segments of foreign DNA are integrated between short repeat palindromic sequences. These loci are transcribed and the RNA transcripts (“pre-crRNA”) are processed into short CRISPR RNAs (crRNAs). There are three types of CRISPR/Cas systems which all incorporate these RNAs and proteins known as “Cas” proteins (CRISPR associated). Types I and III both have Cas endonucleases that process the pre-crRNAs, that, when fully processed into crRNAs, assemble a multi-Cas protein complex that is capable of cleaving nucleic acids that are complementary to the crRNA.

In type II systems, crRNAs are produced using a different mechanism where a trans-activating RNA (tracrRNA) complementary to repeat sequences in the pre-crRNA, triggers processing by a double strand-specific RNase III in the presence of a Cas9 protein or a variant thereof. Cas9 is then able to cleave a target DNA that is complementary to the mature crRNA however cleavage by Cas9 is dependent both upon base-pairing between a crRNA and a target DNA, and on presence of a short motif in a crRNA referred to as a PAM sequence (protospacer adjacent motif) (see Qi et al (2013) Cell 152:1173). In addition, a tracrRNA must also be present as it base pairs with a crRNA at its 3′ end, and this association triggers Cas9 activity.

A Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The variants of Cas9 are art-recognized, e.g., Cas9 nickase mutant that reduces off-target activity (see e.g., Ran et al. (2014) (ell 154 (6): 1380-1389), nCas, Cas9-D10A.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). Thus, exogenously introduced CRISPR endonuclease (e.g., Cas9 or a variant thereof) and a guide RNA (e.g., sgRNA or gRNA) can induce a DNA break at a specific locus within the genome of a target cell. Non-limiting examples of single-guide RNA or guide RNA (sgRNA or gRNA) sequences suitable for targeting are shown in Table 1 in US Application 2015/0056705, which is incorporated herein in its entirety by reference. In addition, a sgRNA or gRNA may comprise a sequence of GSH loci described herein, including those in Table 2A and Table 2B.

In some embodiments, the gene editing nucleic acid sequence encodes a gene editing nucleic acid molecule selected from the group consisting of: a sequence specific nuclease, one or more guide RNA (gRNA), CRISPR/Cas, a ribonucleoprotein (RNP) or any combination thereof. In some embodiments, the sequence-specific nuclease comprises: a TAL-nuclease, a zinc-finger nuclease (ZFN), a meganuclease, a megaTAL, or an RNA guide endonuclease of a CRISPR/Cas system (e.g., Cas proteins e.g. CAS 1-9, Csy, Cse, Cpf1, Cmr, Csx, Csf, cpfl, nCAS, or others). These gene editing systems are known to those of skill in the art, See for example, TALENS described in International Patent Application No. PCT/US2013/038536, and U.S. Patent Publication No. 2017-0191078-A9 which are incorporated by reference in their entirety. CRISPR cas9 systems are known in the art and described in U.S. patent application Ser. No. 13/842,859 filed on March 2013, and U.S. Pat. Nos. 8,697,359, 8,771,945, 8,795,965, 8,865,406, 8,871,445. A recombinant virion described herein is also useful for deactivated nuclease systems, such as CRISPRi or CRISPRa dCas systems, nCas, or Cas13 systems.

Guide RNAs (gRNAS)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds to a target sequence and e.g., a CRISPR associated protein that can form a ribonucleoprotein (RNP), for example, a CRISPR/Cas complex.

In some embodiments, a guide RNA (gRNA) sequence comprises a targeting sequence that directs the gRNA sequence to a desired site in the genome, is fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq.

A guide sequence can be selected to target any target sequence. In some embodiments, the target sequence is a sequence within a genome of a cell or within a GSH as disclosed herein. In some embodiments, the guide RNA can be complementary to either strand of the targeted DNA sequence. It is appreciated by one of skill in the art that for the purposes of targeted cleavage by an RNA-guided endonuclease, target sequences that are unique in the genome are preferred over target sequences that occur more than once in a genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer et al. “E-CRISP: fast CRISPR target site identification” Nat. Methods 11:122-123 (2014); Bae et al. “Cas-OFFinder: a fast and versatile algorithm that searches for potential off-target sites of Cas9 RNA-guided endonucleases” Bioinformatics 30 (10): 1473-1475 (2014); Aach et al. “CasFinder: Flexible algorithm for identifying specific Cas9 targets in genomes” BioRxiv (2014)).

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to a target sequence as described herein, and (ii) a CRISPR repeat. Similarly, a tracrRNA (“transactivating CRISPR RNA”) portion of the fusion can be designed to comprise a secondary structure similar to the tracrRNA sequences in prokaryotes (e.g., a hairpin), to permit formation of an endonuclease complex. In some embodiments, a single transcript further includes a transcription termination sequence, such as a polyT sequence, for example six T nucleotides. In some embodiments, a guide RNA can comprise two RNA molecules and is referred to herein as a “dual guide RNA” or “dgRNA.” In some embodiments, a dgRNA may comprise a first RNA molecule comprising a crRNA, and a second RNA molecule comprising a tracrRNA. The first and second RNA molecules may form a RNA duplex via the base pairing between the flagpole on a crRNA and a tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, a sgRNA can comprise a crRNA covalently linked to a tracrRNA. In some embodiments, a crRNA and tracrRNA can be covalently linked via a linker. In some embodiments, a sgRNA can comprise a stem-loop structure via the base-pairing between the flagpole on a crRNA and a tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a nucleic acid vector as described herein for integration of a nucleic acid of interest into a GSH loci, or composition thereof comprises a nucleic acid that encodes at least 1 gRNA. For example, a second polynucleotide sequence may encode between 1 gRNA and 50 gRNAs, or any integer between 1-50. Each of the polynucleotide sequences encoding the different gRNAs can be operably linked to a promoter. In some embodiments, promoters that are operably linked to the different gRNAs may be the same promoter. Promoters that are operably linked to different gRNAs may be different promoters. A promoter may be a constitutive promoter, an inducible promoter, a repressible promoter, or a regulatable promoter.

In some embodiments, a nucleic acid for integration into a GSH locus encodes for a recombinant virion comprising the said nucleic acid is administered in conjunction with another virion comprising a nucleic acid that encodes a Cas nickase (nCas; e.g., Cas9 nickase or Cas9-D10A). It is contemplated herein that such an nCas enzyme is used in conjunction with a guide RNA that comprises homology to a GSH as described herein and can be used, for example, to release physically constrained sequences or to provide torsional release. Releasing physically constrained sequences can, for example, “unwind” the vector such that a homology directed repair (HDR) template homology arm(s) are exposed for interaction with the genomic sequence.

In some embodiments, zinc finger nuclease is used to induce a DNA break that facilitates integration of the desired nucleic acid. “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In some embodiments, a nucleic acid for integration described herein is integrated into a target genome in a nuclease-free homology-dependent repair systems, e.g., as described in Porro et al., Promoterless gene targeting without nucleases rescues lethality of a Crigler-Najjar syndrome mouse model, EMBO Molecular Medicine, (2017). In some embodiments, the in vivo gene targeting approaches are suitable for the insertion of a donor sequence, without the use of nucleases. In some embodiments, the donor sequence may be promoterless.

In some embodiments, a nuclease located between the restriction sites can be a RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to a selected target DNA sequence in a GSH identified herein.

### CRISPR CAS Systems

As art-recognized and described above, a CRISPR-CAS9 system includes a combination of protein and ribonucleic acid (“RNA”) that can alter a genetic sequence of an organism (see, e.g., US publication 2014/0170753). CRISPR-Cas9 provides a set of tools for Cas9-mediated genome editing via nonhomologous end joining (NHEJ) or homologous recombination in mammalian cells. One of ordinary skill in the art may select between a number of known CRISPR systems such as Type I, Type II, and Type III. In some embodiments, a nucleic acid described herein for integration of a nucleic acid of interest into a GSH loci can be designed to include sequences encoding one or more components of these systems such as a guide RNA, tracrRNA, or Cas (e.g., Cas9 or a variant thereof). In certain embodiments, a single promoter drives expression of a guide sequence and tracrRNA, and a separate promoter drives Cas (e.g., Cas9 or a variant thereof) expression. One of skill in the art will appreciate that certain Cas nucleases require the presence of a protospacer adjacent motif (PAM) adjacent to a target nucleic acid sequence.

RNA-guided nucleases including Cas (e.g., Cas9 or a variant thereof) are suitable for initiating and/or facilitating the integration of a nucleic acid delivered by a recombinant virion described herein. The guide RNAs can be directed to the same strand of DNA or the complementary strand.

In some embodiments, methods and compositions described herein can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9 or a variant thereof) that cannot generate a double strand break (DSB). This permits an endonuclease, in combination with guide RNAs, to bind specifically to a target sequence in a genome and provide RNA-directed reversible transcriptional control.

Accordingly, in some embodiments, a nucleic acid compositions and methods described herein for integration of a nucleic acid of interest into a GSH locus can comprise a deactivated endonuclease, e.g., RNA-guided endonuclease and/or Cas9 or a variant thereof, wherein the deactivated endonuclease lacks endonuclease activity, but retains the ability to bind DNA in a site-specific manner, e.g., in combination with one or more guide RNAs and/or sgRNAs. In some embodiments, a vector can further comprise one or more tracrRNAs, guide RNAs, or sgRNAs. In some embodiments, a de-activated endonuclease can further comprise a transcriptional activation domain.

In some embodiments, a nucleic acid compositions and methods described herein for integration of a nucleic acid of interest into a GSH locus can comprise a hybrid recombinase. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases include those described in Gaj et al. Enhancing the Specificity of Recombinase-Mediated Genome Engineering through Dimer Interface Redesign, Journal of the American Chemical Society, (2014).

Nucleases described herein can be altered, e.g., engineered to design sequence specific nuclease (see, e.g., U.S. Pat. No. 8,021,867). Nucleases can be designed using the methods described in e.g., Certo et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

### MegaTALs

In some embodiments, a nuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42 (4): 2591-601 and Boissel 2015 Methods Mol Biol 1239:171-196. Protocols for megaTAL-mediated gene knockout and gene editing are known in the art, see, e.g., Sather et al. Science Translational Medicine 2015 7 (307): ral56 and Boissel et al. 2014 Nucleic Acids Research 42 (4): 2591-601. MegaTALs can be used as an alternative endonuclease in any of the methods and compositions described herein.

### Marker/Reporter Genes

Exemplary marker genes include but not limited to any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluo-rescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyan1, Midoriishi-Cyan) red fluorescent proteins (e.g., mKate, mKate2, mPlum, DsRed monomer, mCherry, mRFP1, DsRed-Express, DsRed2, HcRed-Tandem, HcRed 1, AsRed2, eqFP61 1, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

Marker genes may also include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. In some embodiments, where the marker gene is green fluorescent protein or luciferase, the vector carrying the signal may be measured colorimetrically based on visible light absorbance or light production in a luminometer, respectively. Such reporters can, for example, be useful in verifying the tissue-specific targeting capabilities and tissue specific promoter regulatory activity of a nucleic acid.

Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase).

### Nucleic Acids

### Non-Coding RNA & Coding RNA

In some embodiments, a nucleic acid of interest encodes a receptor, toxin, a hormone, an enzyme, a marker protein encoded by a marker gene (see above), or a cell surface protein or a therapeutic protein, peptide or antibody or fragment thereof. In some embodiments, a nucleic acid of interest for use in vector compositions as disclosed herein encodes any polypeptide of which expression in a cell is desired, including, but not limited to antibodies, antigens, enzymes, receptors (cell surface or nuclear), hormones, lymphokines, cytokines, reporter polypeptides, growth factors, and functional fragments of any of the above.

In some embodiments, a nucleic acid of interest for use in a recombinant virion as disclosed herein encodes a polypeptide that is lacking or non-functional in a subject having a disease, including but not limited to any of the diseases described herein. In some embodiments, a disease is a genetic disease.

In some aspects, a nucleic acid of interest as defined herein encodes a nucleic acid for use in methods of preventing or treating one or more genetic deficiencies or dysfunctions in a mammal, such as for example, a polypeptide deficiency or polypeptide excess in a mammal, and particularly for preventing, treating, and/or reducing the severity or extent of deficiency in a human manifesting one or more of the disorders linked to a deficiency in such polypeptides in cells and tissues. The method involves administration of a nucleic acid of interest (e.g., a nucleic acid as described by the disclosure) that encodes one or more therapeutic peptides, polypeptides, siRNAs, microRNAs, antisense nucleotides, etc. packaged in a recombinant virion described herein, preferably in a pharmaceutically acceptable composition, to the subject in an amount and for a period of time sufficient to prevent or treat the deficiency or disorder in a subject suffering from such a disorder.

Thus, in some embodiments, nucleic acids of interest for use in vector compositions as disclosed herein can encode one or more peptides, polypeptides, or proteins, which are useful for treatment or prevention of a disease in a mammalian subject.

Exemplary nucleic acids of interest for use in the compositions and methods as disclosed herein include but not limited to: BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16, IL-17, IL-18, VEGF, FGF, SDF-1, connexin 40, connexin 43, SCN4a, HIFia, SERCa2a, ADCY1, and ADCY6.

In some embodiments, a nucleic acid may comprise a coding sequence or a fragment thereof selected from the group consisting of a mammalian β globin gene (e.g., HBA1, HBA2, HBB, HBG1, HBG2, HBD, HBE1, and/or HBZ), alpha-hemoglobin stabilizing protein (AHSP), a B-cell lymphoma/leukemia 11A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (HTT) gene, a rhodopsin (RHO) gene, a Cystic Fibrosis Transmembrane Conductance Regulator (CFTR) gene, F8 or a fragment thereof (e.g., fragment encoding B-domain deleted polypeptide (e.g., VIII SQ, p-VIII)), a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CUT A) gene, a dystrophin gene (DMD), a glucocorticoid receptor gene (GR), an IL2RG gene, an RFX5 gene, a FAD2 gene, a FAD3 gene, a ZP15 gene, a KASII gene, a MDH gene, and/or an EPSPS gene.

In some embodiments, a nucleic acid of interest for use in a recombinant virion disclosed herein can be used to restore the expression of genes that are reduced in expression, silenced, or otherwise dysfunctional in a subject. Similarly, in some embodiments, a nucleic acid of interest for use in a recombinant virion disclosed herein can also be used to knockdown the expression of genes that are aberrantly expressed in a subject.

In some embodiments, a dysfunctional gene is a tumor suppressor that has been silenced in a subject having cancer. In some embodiments, a dysfunctional gene is an oncogene that is aberrantly expressed in a subject having a cancer. Exemplary genes associated with cancer (oncogenes and tumor suppressors) include but not limited to: AARS, ABCB 1, ABCC4, ABI2, ABL1, ABL2, ACK1, ACP2, ACY1, ADSL, AK1, AKR1C2, AKT1, ALB, ANPEP, ANXAS, ANXA7, AP2M1, APC, ARHGAPS, ARHGEFS, ARID4A, ASNS, ATF4, ATM, ATPSB, ATPSO, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCTS, CCYR61, CD24, CD44, CD59, CDC20, CDC25, CDC25A, CDC25B, CDC2LS, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZDS, FZD9, G22P1, GAS6, GCNSL2, GDF1S, GNA13, GNAS, GNB2, GNB2L1, GPR39, GRB2, GSK3A, GSPT1, GTF21, HDAC1, HDGF, HMMR, HPRT1, HRB, HSPA4, HSPAS, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBPS, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB I, NFKB2, NFSF7, NID, NINJ1, NMBR, NME1, NME2, NME3, NOTCH 1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2RSA, PRDX2, PRDX4, PRKAR1A, PRKCBP1, PRNP, PRSS15, PSMA1, PTCH, PTEN, PTGS1, PTMA, PTN, PTPRN, RABSA, RAC1, RADSO, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6KB 1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBR1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYR03, UBC, UBE2L6, UCHL1, USP7, VDAC1, VEGF, VHL, VIL2, WEE1, WNT1, WNT2, WNT2B, WNT3, WNTSA, WT1, XRCC 1, YES 1, YWHAB, YWHAZ, ZAP70, and ZNF9.

In some embodiments, a dysfunctional gene is HBB. In some embodiments, HBB comprises at least one nonsense, frameshift, or splicing mutation that reduces or eliminates the β-globin production. In some embodiments, HBB comprises at least one mutation in the promoter region or polyadenylation signal of HBB. In some embodiments, an HBB mutation is at least one of c. 17A>T, c.−1360G, c.92+1G>A, c.92+6T>C, c.93-21G>A, c. 1180T, c.316-106OG, c.25 26delAA, c.27 28insG, c.92+5G>C, c.1180T, c. 135delC, c.315+1G>A, c.−78A>G, c.52A>T, c.59A>G, c.92+5G>C, c. 124_127delTTCT, c.316-1970T, c.−78A>G, c.52A>T, c. 124_127delTTCT, c.316-197C>T, C.−1380T, c.−79A>G, c.92+5G>C, c.75T>A, c.316-2A>G, and c.316-2A>C.

In certain embodiments, the sickle cell disease is improved by gene therapy (e.g., stem cell gene therapy) that introduces an HBB variant that comprises one or more mutations comprising anti-sickling activity. In some embodiments, an HBB variant may be a double mutant (βAS2; T87Q and E22A). In other embodiments, an HBB variant may be a triple-mutant β-globin variant (βAS3; T87Q, E22A, and G16D). A modification at β16, glycine to aspartic acid, serves a competitive advantage over sickle globin (βS, HbS) for binding to a chain. A modification at β22, glutamic acid to alanine, partially enhances axial interaction with α20 histidine. These modifications result in anti-sickling properties greater than those of the single T87Q-modified variant and comparable to fetal globin. In a SCD murine model, transplantation of bone marrow stem cells transduced with SIN lentivirus carrying βAS3 reversed the red blood cell physiology and SCD clinical symptoms. Accordingly, this variant is being tested in a clinical trial (Identifier no: NCT02247843), Cytotherapy (2018) 20 (7): 899-910.

In some embodiments, a dysfunctional gene is CFTR. In some embodiments, CFTR comprises a mutation selected from ΔF508, R553X, R74W, R668C, S977F, L997F, K1060T, A1067T, R1070Q, R1066H, T3381, R334W, G85E, A46D, 1336K, H1054D, M1V, E92K, V520F, H1085R, R560T, L927P, R560S, N1303K, M1101K, L1077P, R1066M, R1066C, L1065P, Y569D, A561E, A559T, S492F, L467P, R347P, S341P, 1507del, G1061R, G542X, W1282X, and 2184InsA.

In some embodiments, a nucleic acid of interest as defined herein encodes a small interfering nucleic acid (e.g., shRNAs, miRNAs) that inhibits the expression of a gene product associated with cancer (e.g., oncogenes) may be used to prevent or treat the cancer. In some embodiments, a nucleic acid of interest as defined herein encodes a gene product associated with cancer (or a functional RNA that inhibits the expression of a gene associated with cancer) for use, e.g., for research purposes, e.g., to study the cancer or to identify therapeutics that prevent or treat the cancer.

An ordinarily skilled artisan also appreciates that a nucleic acids of interest can comprise one or more mutations that result in conservative amino acid substitutions which may provide functionally equivalent variants, or homologs of a protein or polypeptide. Additionally contemplated in this disclosure is a nucleic acid of interest in a recombinant virion described herein, having a dominant negative mutation. For example, a nucleic acid of interest can encode a mutant protein that interacts with the same elements as a wild-type protein, and thereby blocks some aspects of the function of the wild-type protein.

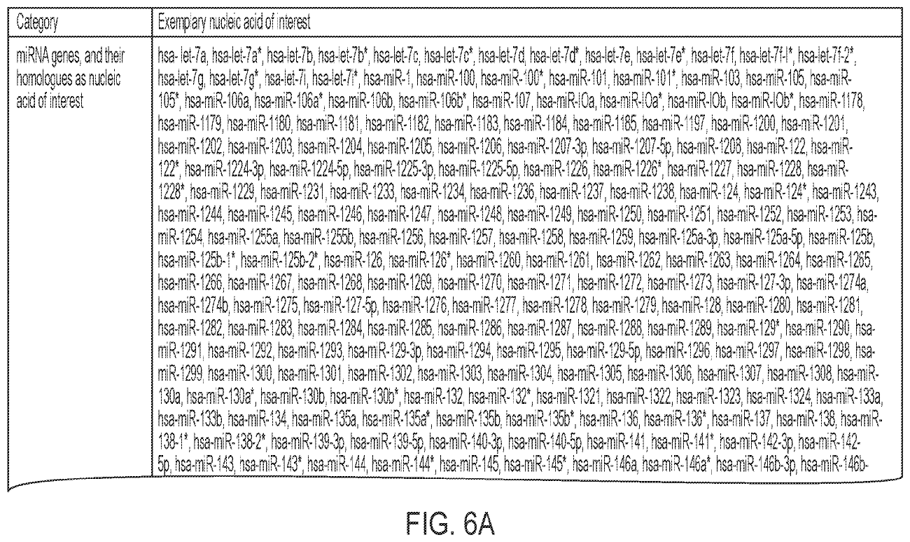

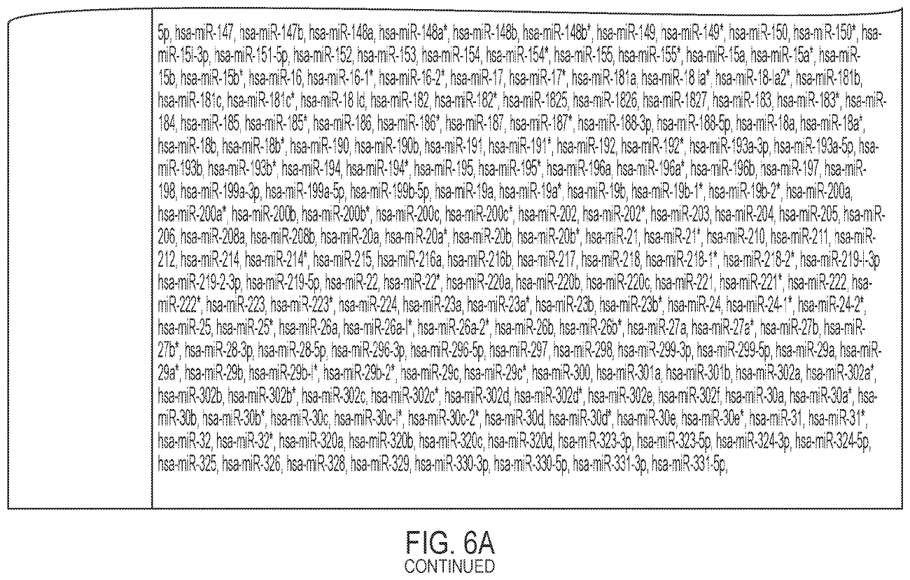

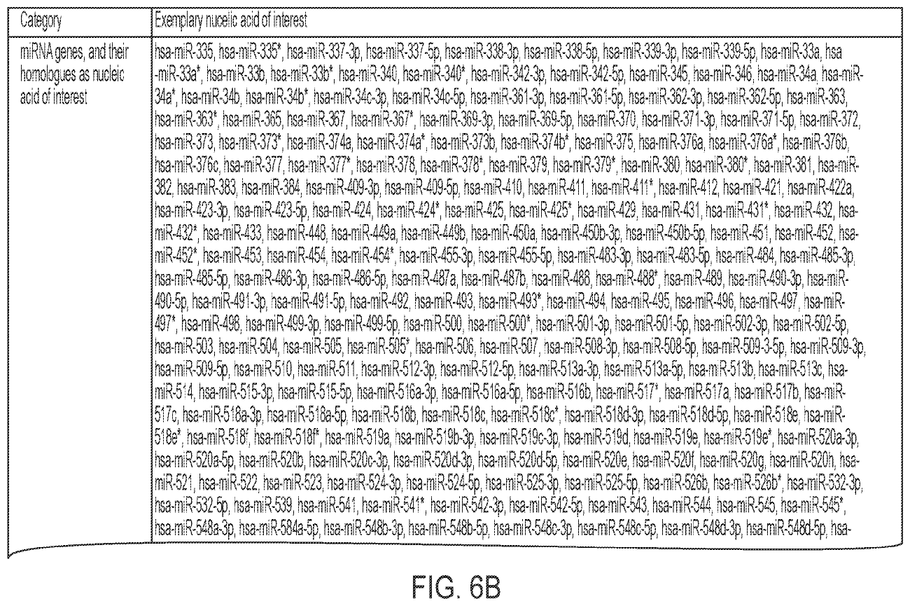

In some embodiments, a nucleic acid of interest in a recombinant virion disclosed herein includes miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA. FIG. 6A and FIG. 6B disclose a non-limiting list of miRNA genes, and their homologues, or as targets for small interfering nucleic acids encoded by a nucleic acid described herein (e.g., miRNA sponges, antisense oligonucleotides, TuD RNAs).

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (de-repress the polypeptide). In some embodiments, de-repression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

### Regulatory Sequences

A nucleic acid of a recombinant virion disclosed herein may also comprise transcriptional or translational regulatory sequences, for example, promoters, enhancers, insulators, internal ribosome entry sites, sequences encoding 2A peptides and/or polyadenylation signals.

In some embodiments, a regulatory sequence includes a suitable promoter sequence, being able to direct transcription of a gene operably linked to a promoter sequence, such as a nucleic acid of interest as described herein. In embodiments, an enhancer sequence is provided upstream of a promoter to increase the efficacy of a promoter. In some embodiments, a regulatory sequence includes an enhancer and a promoter, wherein a second nucleotide sequence includes an intron sequence upstream of a nucleotide sequence encoding a nuclease, wherein a intron includes one or more nuclease cleavage site(s), and wherein a promoter is operably linked to a nucleotide sequence encoding a nuclease.

Suitable promoters, including those described herein, can be derived from viruses and can therefore be referred to as viral promoters, or they can be derived from any organism, including prokaryotic or eukaryotic organisms. In some embodiments, promoters are derived from insect cells or mammalian cells. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (Miyagishi et al., Nature Biotechnology 20, 497-500 (2002)), an enhanced U6 promoter (e.g., Xia et al.,

Nucleic Acids Res. 2003 Sep. 1; 31 (17)), a human H1 promoter (H1), and the like. In some embodiments, these promoters are altered to include one or more nuclease cleavage sites.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate expression of a gene component constitutively, or differentially with respect to cell, a tissue or organ in which expression occurs or, with respect to a developmental stage at which expression occurs, or in response to external stimuli such as physiological stresses, pathogens, metal ions, or inducing agents. Representative examples of promoters include a bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as promoters listed below. Such promoters and/or enhancers can be used for expression of any gene of interest, e.g., the gene editing molecules, donor sequence, therapeutic proteins etc.). For example, a nucleic acid may comprise a promoter that is operably linked to a DNA endonuclease or CRISPR/Cas9-based system. A promoter operably linked to the CRISPR/Cas9-based system or a site-specific nuclease coding sequence may be a promoter from simian virus 40 (SV40), a CAG promoter, a mouse mammary tumor virus (MMTV) promoter, a human immunodeficiency virus (HIV) promoter such as a bovine immunodeficiency virus (BIV) long terminal repeat (LTR) promoter, a Moloney virus promoter, an avian leukosis virus (ALV) promoter, a cytomegalovirus (CMV) promoter such as a CMV immediate early promoter, Epstein Barr virus (EBV) promoter, or a Rous sarcoma virus (RSV) promoter. A promoter may also be a promoter from a human gene such as human ubiquitin C (hUbC), human actin, human myosin, human hemoglobin, human muscle creatine, or human metalothionein. A promoter may also be a tissue specific promoter, such as a liver specific promoter, natural or synthetic. In one embodiment, delivery to a liver can be achieved using endogenous ApoE specific targeting of the composition comprising a vector to hepatocytes via the low density lipoprotein (LDL) receptor present on the surface of the hepatocyte. In some embodiments, use is made of in silico designed synthetic promoters having an assembly of regulatory elements. These synthetic promoters are not naturally occurring and are designed either for optimal expression in a target tissue, regulated expression, or for accommodation in a virus capsid.

In some embodiments, a promoter may be selected from: (a) a promoter heterologous to a nucleic acid, (b) a promoter that facilitates the tissue-specific expression of a nucleic acid, preferably wherein the promoter facilitates hematopoietic cell-specific expression or erythroid lineage-specific expression, (c) a promoter that facilitates the constitutive expression of a nucleic acid, and (d) a promoter that is inducibly expressed, optionally in response to a metabolite or small molecule or chemical entity. Examples of inducible promoters include those regulated by tetracycline, cumate, rapamycin, FKCsA, ABA, tamoxifen, blue light, and riboswitch. Additional details are provided in e.g., Kallunki et al. (2019) Cells 8: E796, which is incorporated by reference. In some embodiments, a promoter is a human erythroparvovirus B19 promoter. In some embodiments, a promoter is not a human erythroparvovirus B19 promoter. In some embodiments, a promoter is selected from the CMV promoter, β-globin promoter, CAG promoter, AHSP promoter, MND promoter, Wiskott-Aldrich promoter, and PKLR promoter.

### Sequences

As used herein, coding region refers to regions of a nucleotide sequence comprising codons which are translated into amino acid residues, whereas noncoding region refers to regions of a nucleotide sequence that are not translated into amino acids. Transcribed non-coding sequences may be upstream (5′-UTR), downstream (3′-UTR), or intronic. Non-transcribed non-coding sequences may have cis-acting. regulatory functions, e.g., enhancer and promoter, or act as “spacers,” non-transcribed DNA used to separate functional groups in the DNA, e.g., polylinkers or “stuffer” DNA used to increase the size of a vector genome.

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

There is a known and definite correspondence between an amino acid sequence of a particular protein and nucleotide sequences that can code for the protein, as defined by the genetic code (shown below). Likewise, there is a known and definite correspondence between a nucleotide sequence of a particular nucleic acid and an amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its degeneracy, whereby, for most of the amino acids used to make proteins, more than one coding nucleotide triplet may be employed (illustrated above). Therefore, a number of different nucleotide sequences may code for a given amino acid sequence. The universality of the genetic code provides that such nucleotide sequences are considered functionally equivalent since they result in the production of the same amino acid sequence in all organisms, although mitochondria and plastids and similar symbiotic organelles have a slightly different genetic code. Although not all codons are utilized with similar translation efficiency, rare codons may lower the protein production due to limiting tRNA pools. Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (<RTI3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

It is also known in the art that a nucleic acid encoding a polypeptide can be codon-optimized for certain host cells, without altering the amino acid sequence. Codon-optimization describes gene engineering approaches that use synonymous codon changes to increase protein production. This is possible because most amino acids are encoded by more than one codon. Replacing rare codons with frequently used ones have shown to increase protein expression.

In view of the foregoing, a nucleotide sequence of a DNA or RNA encoding a nucleic acid (or any portion thereof) described herein (e.g., a therapeutic nucleic acid) can be used to derive a polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well-known in the art and readily available on publicly available databases, such as the National Center for Biotechnology Information (NCBI).

### Representative Sequences

SEQ ID NO: 1, 2, and 3—represent examples of AAV ITRs. Lower case font is non-ITR sequence; [symbol] terminal resolution site (trs); [symbol] —A and A′ stem; solid underline—B/B′ and C/C′ stems.

SEQ ID NO: 6, 7, and 8—open reading frame for B19 VP1 and VP2. The initiation codons are in bold and underlined. The minor capsid protein, VP1, utilizes a non-canonical start codon and the major coat protein, VP2, utilizes the conventional initiation ATG triplet.

AAV ITR “Flip” conformer nucleic acid sequence
SEQ ID NO: 1

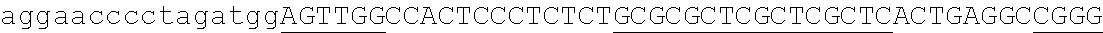

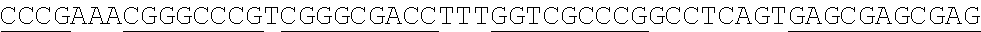

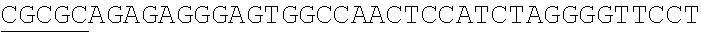

AAV ITR “Flip” conformer nucleic acid sequence
SEQ ID NO: 2
cctgcaggcagCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTG

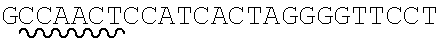

AAV ITR “Flop” conformer nucleic acid sequence
SEQ ID NO: 3

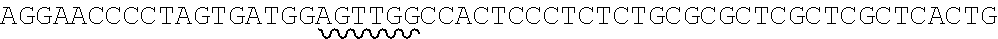

AGGCCGGGCGACCAAAGGTCGCCCGACGCCCGGGCTTTGCCCGGGCGGCCTCAGTG
AGCGAGCGAGCGCGCAGctgcctgcagg
B19 inverted terminal repeat (ITR) 5′ end nucleic acid sequence
SEQ ID NO: 4
CCAAATCAGATGCCGCCGGTCGCCGCCGGTAGGCGGGACTTCCGGTACAAGATGGC
GGACAATTACGTCATTTCCTGTGACGTCATTTCCTGTGACGTCACTTCCGGTGGGCG
GGACTTCCGGAATTAGGGTTGGCTCTGGGCCAGCGCTTGGGGTTGCCTTGACACTAA
GACAAGCGGCGCGCCGCTTGATCTTAGTGGCACGTCAACCCCAAGCAAGCTGGCCC
AGAGCCAACCCTAATTCCGGAAGTCCCGCCCACCGGAAGTGACGTCACAGGAAATG
ACGTCACAGGAAATGACGTAATTGTCCGCCATCTTGTACCGGAAGTCCCGCCTACCG
GCGGCGACCGGCGGCATCTGATTTGGTGTCTTCTTTTAAATTTT
B19 inverted terminal repeat (ITR) 3′ end nucleic acid sequence
SEQ ID NO: 5
AAAATTTAAAAGAAGACACCAAATCAGATGCCGCCGGTCGCCGCCGGTAGGCGGGA
CTTCCGGTACAAGATGGCGGACAATTACGTCATTTCCTGTGACGTCATTTCCTGTGA
CGTCACTTCCGGTGGGGGGACTTCCGGAATTAGGGTTGGCTCTGGGCCAGCTTGCT
TGGGGTTGACGTGCCACTAAGATCAAGCGGCGCGCCGCTTGTCTTAGTGTCAAGGCA
ACCCCAAGCGCTGGCCCAGAGCCAACCCTAATTCCGGAAGTCCCGCCCACCGGAAG
TGACGTCACAGGAAATGACGTCACAGGAAATGACGTAATTGTCCGCCATCTTGTACC
GGAAGTCCCGCCTACCGGCGGCGACCGGCGGCATCTGATTTGG
B19 VP1-TTG & VP2-ATG nucleic acid sequence
SEQ ID NO: 6
ACTCGACGAAGACTTGATCACCCGGGGGATCCTGTTAAATTGAGTAAAGAAAGTGG
CAAATGGTGGGAAAGTGATGATGAATTTGCTAAAGCTGTGTATCAGCAATTTGTGGA
ATTTTATGAAAAGGTTACTGGAACAGACTTAGAGCTTATTCAAATATTAAAAGATCA
TTATAATATTTCTTTAGATAATCCCCTAGAAAACCCATCCTCTCTGTTTGACTTAGTT
GCTCGCATTAAAAATAACCTTAAAAATTCTCCAGACTTATATAGTCATCATTTTCAA
AGTCATGGACAGTTATCTGACCACCCCCATGCCTTATCATCCAGTAGCAGTCATGCA
GAACCTAGAGGAGAAGATGCAGTATTATCTAGTGAAGACTTACACAAGCCTGGGCA
AGTTAGCGTACAACTACCCGGTACTAACTATGTTGGGCCTGGCAATGAGCTACAAGC
TGGGCCCCCGCAAAGTGCTGTTGACAGTGCTGCAAGGATTCATGACTTTAGGTATAG
CCAACTCGCTAAGCTCGGAATAAATCCATATACTCATTGGACTGTAGCAGATGAAGA
GCTTTTAAAAAATATAAAAAATGAAACTGGGTTTCAAGCACAAGTAGTAAAAGACT
ACTTTACTTTAAAAGGTGCAGCTGCCCCTGTGGCCCATTTTCAAGGAAGTTTGCCGG
AAGTTCCCGCTTACAACGCCTCAGAAAAATACCCAAGCATGACTTCAGTTAATTCTG
CAGAAGCCAGCACTGGTGCAGGAGGGGGGGGCAGTAATCCTGTCAAAAGCATGTGG
AGTGAGGGGGCCACTTTTAGTGCCAACTCTGTGACTTGTACATTTTCCAGGCAGTTTT
TAATTCCATATGACCCAGAGCACCATTATAAGGTGTTTTCTCCCGCAGCAAGTAGCT
GCCACAATGCCAGTGGAAAGGAGGCAAAGGTTTGCACCATTAGTCCCATAATGGGA
TACTCAACCCCATGGAGATATTTAGATTTTAATGCTTTAAACTTATTTTTTTCACCTTT
AGAGTTTCAGCACTTAATTGAAAATTATGGAAGTATAGCTCCTGATGCTTTAACTGT
AACCATATCAGAAATTGCTGTTAAGGATGTTACAGACAAAACTGGAGGGGGGGTGC
AGGTTACTGACAGCACTACAGGGCGCCTATGCATGTTAGTAGACCATGAATACAAG
TACCCATATGTGTTAGGGCAAGGTCAAGATACTTTAGCCCCAGAACTTCCTATTTGG
GTCTACTTTCCCCCTCAATATGCTTACTTAACAGTAGGAGATGTTAACACACAAGGA
ATTTCTGGAGACAGCAAAAAATTAGCAAGTGAAGAATCAGCATTTTATGTTTTGGAA
CACAGTTCTTTTCAGCTTTTAGGTACAGGAGGTACAGCAACTATGTCTTATAAGTTTC
CTCCAGTGCCCCCAGAAAATTTAGAGGGCTGCAGTCAACACTTTTATGAGATGTACA
ATCCCTTATACGGATCCCGCTTAGGGGTTCCTGACACATTAGGAGGTGACCCAAAAT
TTAGATCTTTAACACATGAAGACCATGCAATTCAGCCCCAAAACTTCATGCCAGGGC
CACTAGTAAACTCAGTGTCTACAAAGGAGGGAGACAGCTCTAATACTGGAGCTGGG
AAAGCCTTAACAGGCCTTAGCACAGGTACCTCTCAAAACACTAGAATATCCTTACGC
CCGGGGCCAGTGTCTCAGCCGTACCACCACTGGGACACAGATAAATATGTCACAGG
AATAAATGCTATTTCTCATGGTCAGACCACTTATGGTAACGCTGAAGACAAAGAGTA
TCAGCAAGGAGTGGGTAGATTTCCAAATGAAAAAGAACAGCTAAAACAGTTACAGG
GTTTAAACATGCACACCTACTTTCCCAATAAAGGAACCCAGCAATATACAGATCAAA
TTGAGCGCCCCCTAATGGTGGGTTCTGTATGGAACAGAAGAGCCCTTCACTATGAAA
GCCAGCTGTGGAGTAAAATTCCAAATTTAGATGACAGTTTTAAAACTCAGTTTGCAG
CCTTAGGAGGATGGGGTTTGCATCAGCCACCTCCTCAAATATTTTTAAAAATATTAC
CACAAAGTGGGCCAATTGGAGGTATTAAATCAATGGGAATTACTACCTTAGTTCAGT
ATGCCGTGGGAATTATGACAGTAACCATGACATTTAAATTGGGGCCCCGTAAAGCTA
CGGGACGGTGGAATCCTCAACCTGGAGTATATCCCCCGCACGCAGCAGGTCATTTAC
CATATGTACTATATGACCCTACAGCTACAGATGCAAAACAACACCACAGACATGGA
TATGAAAAGCCTGAAGAATTGTGGACAGCCAAAAGCCGTGTGCACCCATTGtaa
B19 VP1-CTG & VP2-ATG nucleic acid sequence
SEQ ID NO: 7
ACTCGACGAAGACTTGATCACCCGGGGGATCCTGTTAAACTGAGTAAAGAAAGTGG
CAAATGGTGGGAAAGTGATGATGAATTTGCTAAAGCTGTGTATCAGCAATTTGTGGA
ATTTTATGAAAAGGTTACTGGAACAGACTTAGAGCTTATTCAAATATTAAAAGATCA
TTATAATATTTCTTTAGATAATCCCCTAGAAAACCCATCCTCTCTGTTTGACTTAGTT
GCTCGCATTAAAAATAACCTTAAAAATTCTCCAGACTTATATAGTCATCATTTTCAA
AGTCATGGACAGTTATCTGACCACCCCCATGCCTTATCATCCAGTAGCAGTCATGCA
GAACCTAGAGGAGAAGATGCAGTATTATCTAGTGAAGACTTACACAAGCCTGGGCA
AGTTAGCGTACAACTACCCGGTACTAACTATGTTGGGCCTGGCAATGAGCTACAAGC
TGGGCCCCCGCAAAGTGCTGTTGACAGTGCTGCAAGGATTCATGACTTTAGGTATAG
CCAACTCGCTAAGCTCGGAATAAATCCATATACTCATTGGACTGTAGCAGATGAAGA
GCTTTTAAAAAATATAAAAAATGAAACTGGGTTTCAAGCACAAGTAGTAAAAGACT
ACTTTACTTTAAAAGGTGCAGCTGCCCCTGTGGCCCATTTTCAAGGAAGTTTGCCGG
AAGTTCCCGCTTACAACGCCTCAGAAAAATACCCAAGCATGACTTCAGTTAATTCTG
CAGAAGCCAGCACTGGTGCAGGAGGGGGGGGCAGTAATCCTGTCAAAAGCATGTGG
AGTGAGGGGGCCACTTTTAGTGCCAACTCTGTGACTTGTACATTTTCCAGGCAGTTTT
TAATTCCATATGACCCAGAGCACCATTATAAGGTGTTTTCTCCCGCAGCAAGTAGCT
GCCACAATGCCAGTGGAAAGGAGGCAAAGGTTTGCACCATTAGTCCCATAATGGGA
TACTCAACCCCATGGAGATATTTAGATTTTAATGCTTTAAACTTATTTTTTTCACCTTT
AGAGTTTCAGCACTTAATTGAAAATTATGGAAGTATAGCTCCTGATGCTTTAACTGT
AACCATATCAGAAATTGCTGTTAAGGATGTTACAGACAAAACTGGAGGGGGGGTGC
AGGTTACTGACAGCACTACAGGGCGCCTATGCATGTTAGTAGACCATGAATACAAG
TACCCATATGTGTTAGGGCAAGGTCAAGATACTTTAGCCCCAGAACTTCCTATTTGG
GTCTACTTTCCCCCTCAATATGCTTACTTAACAGTAGGAGATGTTAACACACAAGGA
ATTTCTGGAGACAGCAAAAAATTAGCAAGTGAAGAATCAGCATTTTATGTTTTGGAA
CACAGTTCTTTTCAGCTTTTAGGTACAGGAGGTACAGCAACTATGTCTTATAAGTTTC
CTCCAGTGCCCCCAGAAAATTTAGAGGGCTGCAGTCAACACTTTTATGAGATGTACA
ATCCCTTATACGGATCCCGCTTAGGGGTTCCTGACACATTAGGAGGTGACCCAAAAT
TTAGATCTTTAACACATGAAGACCATGCAATTCAGCCCCAAAACTTCATGCCAGGGC
CACTAGTAAACTCAGTGTCTACAAAGGAGGGAGACAGCTCTAATACTGGAGCTGGG
AAAGCCTTAACAGGCCTTAGCACAGGTACCTCTCAAAACACTAGAATATCCTTACGC
CCGGGGCCAGTGTCTCAGCCGTACCACCACTGGGACACAGATAAATATGTCACAGG
AATAAATGCTATTTCTCATGGTCAGACCACTTATGGTAACGCTGAAGACAAAGAGTA
TCAGCAAGGAGTGGGTAGATTTCCAAATGAAAAAGAACAGCTAAAACAGTTACAGG
GTTTAAACATGCACACCTACTTTCCCAATAAAGGAACCCAGCAATATACAGATCAAA
TTGAGCGCCCCCTAATGGTGGGTTCTGTATGGAACAGAAGAGCCCTTCACTATGAAA
GCCAGCTGTGGAGTAAAATTCCAAATTTAGATGACAGTTTTAAAACTCAGTTTGCAG
CCTTAGGAGGATGGGGTTTGCATCAGCCACCTCCTCAAATATTTTTAAAAATATTAC
CACAAAGTGGGCCAATTGGAGGTATTAAATCAATGGGAATTACTACCTTAGTTCAGT
ATGCCGTGGGAATTATGACAGTAACCATGACATTTAAATTGGGGCCCCGTAAAGCTA
CGGGACGGTGGAATCCTCAACCTGGAGTATATCCCCCGCACGCAGCAGGTCATTTAC
CATATGTACTATATGACCCTACAGCTACAGATGCAAAACAACACCACAGACATGGA
TATGAAAAGCCTGAAGAATTGTGGACAGCCAAAAGCCGTGTGCACCCATTGtaa
B19 VP1-ACG & VP2-ATG nucleic acid sequence
SEQ ID NO: 8
ACTCGACGAAGACTTGATCACCCGGGGGATCCTGTTAAAACGAGTAAAGAAAGTGG
CAAATGGTGGGAAAGTGATGATGAATTTGCTAAAGCTGTGTATCAGCAATTTGTGGA
ATTTTATGAAAAGGTTACTGGAACAGACTTAGAGCTTATTCAAATATTAAAAGATCA
TTATAATATTTCTTTAGATAATCCCCTAGAAAACCCATCCTCTCTGTTTGACTTAGTT
GCTCGCATTAAAAATAACCTTAAAAATTCTCCAGACTTATATAGTCATCATTTTCAA
AGTCATGGACAGTTATCTGACCACCCCCATGCCTTATCATCCAGTAGCAGTCATGCA
GAACCTAGAGGAGAAGATGCAGTATTATCTAGTGAAGACTTACACAAGCCTGGGCA
AGTTAGCGTACAACTACCCGGTACTAACTATGTTGGGCCTGGCAATGAGCTACAAGC
TGGGCCCCCGCAAAGTGCTGTTGACAGTGCTGCAAGGATTCATGACTTTAGGTATAG
CCAACTCGCTAAGCTCGGAATAAATCCATATACTCATTGGACTGTAGCAGATGAAGA
GCTTTTAAAAAATATAAAAAATGAAACTGGGTTTCAAGCACAAGTAGTAAAAGACT
ACTTTACTTTAAAAGGTGCAGCTGCCCCTGTGGCCCATTTTCAAGGAAGTTTGCCGG
AAGTTCCCGCTTACAACGCCTCAGAAAAATACCCAAGCATGACTTCAGTTAATTCTG
CAGAAGCCAGCACTGGTGCAGGAGGGGGGGGCAGTAATCCTGTCAAAAGCATGTGG
AGTGAGGGGGCCACTTTTAGTGCCAACTCTGTGACTTGTACATTTTCCAGGCAGTTTT
TAATTCCATATGACCCAGAGCACCATTATAAGGTGTTTTCTCCCGCAGCAAGTAGCT
GCCACAATGCCAGTGGAAAGGAGGCAAAGGTTTGCACCATTAGTCCCATAATGGGA
TACTCAACCCCATGGAGATATTTAGATTTTAATGCTTTAAACTTATTTTTTTCACCTTT
AGAGTTTCAGCACTTAATTGAAAATTATGGAAGTATAGCTCCTGATGCTTTAACTGT
AACCATATCAGAAATTGCTGTTAAGGATGTTACAGACAAAACTGGAGGGGGGGTGC
AGGTTACTGACAGCACTACAGGGCGCCTATGCATGTTAGTAGACCATGAATACAAG
TACCCATATGTGTTAGGGCAAGGTCAAGATACTTTAGCCCCAGAACTTCCTATTTGG
GTCTACTTTCCCCCTCAATATGCTTACTTAACAGTAGGAGATGTTAACACACAAGGA
ATTTCTGGAGACAGCAAAAAATTAGCAAGTGAAGAATCAGCATTTTATGTTTTGGAA
CACAGTTCTTTTCAGCTTTTAGGTACAGGAGGTACAGCAACTATGTCTTATAAGTTTC
CTCCAGTGCCCCCAGAAAATTTAGAGGGCTGCAGTCAACACTTTTATGAGATGTACA
ATCCCTTATACGGATCCCGCTTAGGGGTTCCTGACACATTAGGAGGTGACCCAAAAT
TTAGATCTTTAACACATGAAGACCATGCAATTCAGCCCCAAAACTTCATGCCAGGGC
CACTAGTAAACTCAGTGTCTACAAAGGAGGGAGACAGCTCTAATACTGGAGCTGGG
AAAGCCTTAACAGGCCTTAGCACAGGTACCTCTCAAAACACTAGAATATCCTTACGC
CCGGGGCCAGTGTCTCAGCCGTACCACCACTGGGACACAGATAAATATGTCACAGG
AATAAATGCTATTTCTCATGGTCAGACCACTTATGGTAACGCTGAAGACAAAGAGTA
TCAGCAAGGAGTGGGTAGATTTCCAAATGAAAAAGAACAGCTAAAACAGTTACAGG
GTTTAAACATGCACACCTACTTTCCCAATAAAGGAACCCAGCAATATACAGATCAAA
TTGAGCGCCCCCTAATGGTGGGTTCTGTATGGAACAGAAGAGCCCTTCACTATGAAA
GCCAGCTGTGGAGTAAAATTCCAAATTTAGATGACAGTTTTAAAACTCAGTTTGCAG
CCTTAGGAGGATGGGGTTTGCATCAGCCACCTCCTCAAATATTTTTAAAAATATTAC
CACAAAGTGGGCCAATTGGAGGTATTAAATCAATGGGAATTACTACCTTAGTTCAGT
ATGCCGTGGGAATTATGACAGTAACCATGACATTTAAATTGGGGCCCCGTAAAGCTA
CGGGACGGTGGAATCCTCAACCTGGAGTATATCCCCCGCACGCAGCAGGTCATTTAC
CATATGTACTATATGACCCTACAGCTACAGATGCAAAACAACACCACAGACATGGA
TATGAAAAGCCTGAAGAATTGTGGACAGCCAAAAGCCGTGTGCACCCATTGtaa
Human erythroparvovirus B19 VP1 amino acid sequence (GenBank:
AAQ91879.1)
SEQ ID NO: 9
MSKESGKWWESDDKFAKAVYQQFVEFYEKVTGTDLELIQILKDHYNISLDNPLENPSSL
FDLVARIKNNLKNSPDLYSHHFQSHGQLSDHPHALSSSSSHAEPRGENAVLSSEDLHKPG
QVSVQLPGTNYVGPGNELQAGPPQSAVDSAARIHDFRYSQLAKLGINPYTHWTVADEE
LLKNIKNETGFQAQVVKDYFTLKGAAAPVAHFQGSLPEVPAYNASEKYPSMTSVNSAE
ASTGAGGGGSNPVKSMWSEGATFSANSVTCTFSRQFLIPYDPEHHYKVFSPAASSCHNA
SGKEAKVCTISPIMGYSTPWRYLDFNALNLFFSPLEFQHLIENYGSIAPDALTVTISEIAVK
DVTDKTGGGVQVTDSTTGRLCMLVDHEYKYPYVLGQGQDTLAPELPIWVYFPPQYAY
LTVGDVNTQGISGDSKKLASEESAFYVLEHSSFQLLGTGGTATMSYKFPPVPPENLEGCS
QHFYEMYNPLYGSRLGVPDTLGGDPKFRSLTHEDHAIQPQNFMPGPLVNSVSTKEGDSS
NTGAGKALTGLSTGTSQNTRISLRPGPVSQPYHHWDTDKYVTGINAISHGQTTYGNAED
KEYQQGVGRFPNEKEQLKQLQGLNMHTYFPNKGTQQYTDQIERPLMVGSVWNRRALH
YESQLWSKIPNLDDSFKTQFAALGGWGLHQPPPQIFLKILPQSGPIGGIKSMGITTLVQYA
VGIMTVTMTFKLGPRKATGRWNPQPGVYPPHAAGHLPYVLYDPTATDAKQHHRHGYE
KPEELWTAKSRVHPL

SEQ ID NO: 10 Human erythroparvovirus B19 VP2 nucleic acid sequence
(GenBank: AY386330.1; CDS 3305..4969)
ATGACTTCAGTTAATTCTGCAGAAGCCAGCACTGGTGCAGGAGGGGGGGGCAGTAA
TCCTGTCAAAAGCATGTGGAGTGAGGGGGCCACTTTTAGTGCCAACTCTGTGACTTG
TACATTTTCTAGACAGTTTTTAATTCCATATGACCCAGAGCACCATTATAAGGTGTTT
TCTCCCGCAGCAAGTAGCTGCCACAATGCCAGTGGAAAGGAGGCAAAGGTTTGCAC
CATTAGTCCCATAATGGGATACTCAACCCCATGGAGATATTTAGATTTTAATGCTTT
AAACTTATTTTTTTCACCTTTAGAGTTTCAGCACTTAATTGAAAATTATGGAAGTATA
GCTCCTGATGCTTTAACTGTAACCATATCAGAAATTGCTGTTAAGGATGTTACAGAC
AAAACTGGAGGGGGGGTGCAGGTTACTGACAGCACTACAGGGCGCCTATGCATGTT
AGTAGACCATGAATACAAGTACCCATATGTGTTAGGGCAAGGTCAAGATACTTTAG
CCCCAGAACTTCCTATTTGGGTATACTTTCCCCCTCAATATGCTTACTTAACAGTAGG
AGATGTTAACACACAAGGAATTTCTGGAGACAGCAAAAAATTAGCAAGTGAAGAAT
CAGCATTTTATGTTTTGGAACACAGTTCTTTTCAGCTTTTAGGTACAGGAGGTACAGC
AACTATGTCTTATAAGTTTCCTCCAGTGCCCCCAGAAAATTTAGAGGGCTGCAGTCA
ACACTTTTATGAGATGTACAATCCCTTATACGGATCCCGCTTAGGGGTTCCTGACAC
ATTAGGAGGTGACCCAAAATTTAGATCTTTAACACATGAAGACCATGCAATTCAGCC
CCAAAACTTCATGCCAGGGCCACTAGTAAACTCAGTGTCTACAAAGGAGGGAGACA
GCTCTAATACTGGAGCTGGGAAAGCCTTAACAGGCCTTAGCACAGGTACCTCTCAAA
ACACTAGAATATCCTTACGCCCGGGGCCAGTGTCTCAGCCGTACCACCACTGGGACA
CAGATAAATATGTCACAGGAATAAATGCTATTTCTCATGGTCAGACCACTTATGGTA
ACGCTGAAGACAAAGAGTATCAGCAAGGAGTGGGTAGATTTCCAAATGAAAAAGA
ACAGCTAAAACAGTTACAGGGTTTAAACATGCACACCTACTTTCCCAATAAAGGAA
CCCAGCAATATACAGATCAAATTGAGCGCCCCCTAATGGTGGGTTCTGTATGGAACA
GAAGAGCCCTTCACTATGAAAGCCAGCTGTGGAGTAAAATTCCAAATTTAGATGAC
AGTTTTAAAACTCAGTTTGCAGCCTTAGGAGGATGGGGTTTGCATCAGCCACCTCCT
CAAATATTTTTAAAAATATTACCACAAAGTGGGCCAATTGGAGGTATTAAATCAATG
GGAATTACTACCTTAGTTCAGTATGCCGTGGGAATTATGACAGTAACCATGACATTT
AAATTGGGGCCCCGTAAAGCTACGGGACGGTGGAATCCTCAACCTGGAGTATATCC
CCCGCACGCAGCAGGTCATTTACCATATGTACTATATGACCCTACAGCTACAGATGC
AAAACAACACCACAGACATGGATATGAAAAGCCTGAAGAATTGTGGACAGCCAAA
AGCCGTGTGCACCCATTGTAA
SEQ ID NO: 11 Human erythroparvovirus B19 VP2 amino acid sequence
(GenBank: AAQ91880.1)
MTSVNSAEASTGAGGGGSNPVKSMWSEGATFSANSVTCTFSRQFLIPYDPEHHYKVFSP
AASSCHNASGKEAKVCTISPIMGYSTPWRYLDFNALNLFFSPLEFQHLIENYGSIAPDALT
VTISEIAVKDVTDKTGGGVQVTDSTTGRLCMLVDHEYKYPYVLGQGQDTLAPELPIWV
YFPPQYAYLTVGDVNTQGISGDSKKLASEESAFYVLEHSSFQLLGTGGTATMSYKFPPV
PPENLEGCSQHFYEMYNPLYGSRLGVPDTLGGDPKFRSLTHEDHAIQPQNFMPGPLVNS
VSTKEGDSSNTGAGKALTGLSTGTSQNTRISLRPGPVSQPYHHWDTDKYVTGINAISHG
QTTYGNAEDKEYQQGVGRFPNEKEQLKQLQGLNMHTYFPNKGTQQYTDQIERPLMVG
SVWNRRALHYESQLWSKIPNLDDSFKTQFAALGGWGLHQPPPQIFLKILPQSGPIGGIKS
MGITTLVQYAVGIMTVTMTFKLGPRKATGRWNPQPGVYPPHAAGHLPYVLYDPTATD
AKQHHRHGYEKPEELWTAKSRVHPL
SEQ ID NO: 12 Human erythroparvovirus B19 ph NS ATG nucleic acid
sequence
TCCGGAATATTAATAGATCATGGAGATAATTAAAATGATAACCATCTCGCAAATAA
ATAAGTATTTTACTGTTTTCGTAACAGTTTTGTAATAAAAAAACCTATAAATATTCCG
GATTATTCATACCGTCCCACCATCGGGCGCGGATCTGCCGCCATGGAGCTATTTAGA
GGGGTGCTTCAAGTTTCTTCTAATGTTCTGGACTGTGCTAACGATAACTGGTGGTGCT
CTTTACTAGATTTAGACACTTCTGACTGGGAACCACTAACTCATACTAACAGACTAA
TGGCAATATACTTAAGCAGTGTGGCTTCTAAGCTTGACCTTACCGGGGGGCCACTAG
CAGGGTGCTTGTACTTTTTTCAAGCAGAATGTAACAAATTTGAAGAAGGCTATCATA
TTCATGTGGTTATTGGGGGGCCAGGGTTAAACCCCAGAAACCTCACAGTGTGTGTAG
AGGGGTTATTTAATAATGTACTTTATCACTTTGTAACTGAAAATGTGAAGCTAAAAT
TTTTGCCAGGAATGACTACAAAAGGCAAATACTTTAGAGATGGAGAGCAGTTTATA
GAAAACTATTTAATGAAAAAAATACCTTTAAATGTTGTATGGTGTGTTACTAATATT
GATGGATATATAGATACCTGTATTTCTGCTACTTTTAGAAGGGGAGCTTGCCATGCC
AAGAAACCCCGCATTACCACAGCCATAAATGATACTAGTAGCGATGCTGGGGAGTC
TAGCGGCACAGGGGCAGAGGTTGTGCCATTTAATGGGAAGGGAACTAAGGCTAGCA
TAAAGTTTCAAACTATGGTAAACTGGTTGTGTGAAAACAGAGTGTTTACAGAGGATA
AGTGGAAACTAGTTGACTTTAACCAGTACACTTTACTAAGCAGTAGTCACAGTGGAA
GTTTTCAAATTCAAAGTGCACTAAAACTAGCAATTTATAAAGCAACTAATTTAGTGC
CTACTAGCACATTTTTATTGCATACAGACTTTGAGCAGGTTATGTGTATTAAAGACA
ATAAAATTGTTAAATTGTTACTTTGTCAAAACTATGACCCCCTATTGGTGGGGCAGC
ATGTGTTAAAGTGGATTGATAAAAAATGTGGCAAGAAAAATACACTGTGGTTTTATG
GGCCGCCAAGTACAGGAAAAACAAACTTGGCAATGGCCATTGCTAAAAGTGTTCCA
GTATATGGCATGGTTAACTGGAATAATGAAAACTTTCCATTTAATGATGTAGCAGGA
AAAAGCTTGGTGGTCTGGGATGAAGGTATTATTAAGTCTACAATTGTAGAAGCTGCA
AAAGCCATTTTAGGCGGGCAACCCACCAGGGTAGATCAAAAAATGCGTGGAAGTGT
AGCTGTGCCTGGAGTACCTGTGGTTATAACCAGCAATGGTGACATTACTTTTGTTGT
AAGCGGGAACACTACAACAACTGTACATGCTAAAGCCTTAAAAGAGCGCATGGTAA
AGTTAAACTTTACTGTAAGATGCAGCCCTGACATGGGGTTACTAACAGAGGCTGATG
TACAACAGTGGCTTACATGGTGTAATGCACAAAGCTGGGACCACTATGAAAACTGG
GCAATAAACTACACTTTTGATTTCCCTGGAATTAATGCAGATGCCCTCCACCCAGAC
CTCCAAACCACCCCAATTGTCACAGACACCAGTATCAGCAGCAGTGGTGGTGAAAG
CTCTGAAGAACTCAGTGAAAGCAGCTTTTTTAACCTCATCACCCCAGGCGCCTGGAA
CACTGAAACCCCGCGCTCTAGTACGCCCATCCCCGGGACCAGTTCAGGAGAATCATT
TGTCGGAAGCCCAGTTTCCTCCGAAGTTGTAGCTGCATCGTGGGAAGAAGCCTTCTA
CACACCTTTGGCAGACCAGTTTCGTGAACTGTTAGTTGGGGTTGATTATGTGTGGGA
CGGTGTAAGGGGTTTACCTGTGTGTTGTGTGCAACATATTAACAATAGTGGGGGAGG
CTTGGGACTTTGTCCCCATTGCATTAATGTAGGGGCTTGGTATAATGGATGGAAATT
TCGAGAATTTACCCCAGATTTGGTGCGATGTAGCTGCCATGTGGGAGCTTCTAATCC
CTTTTCTGTGCTAACCTGCAAAAAATGTGCTTACCTGTCTGGATTGCAAAGCTTTGTA
GATTATGAGTAA
SEQ ID NO: 13 Human erythroparvovirus B19 Non-structural protein NS1
amino acid sequence (GenBank: AAQ91878.1)
MELFRGVLQVSSNVLDCANDNWWCSLLDLDTSDWEPLTHTNRLMAIYLSSVASKLDLT
GGPLAGCLYFFQAECNKFEEGYHIHVVIGGPGLNPRNLTVCVEGLENNVLYHFVTENVK
LKFLPGMTTKGKYFRDGEQFIENYLMKKIPLNVVWCVTNIDGYIDTCISATFRRGACHA
KKPRITTAINDTSSDAGESSGTGAEVVPFNGKGTKASIKFQTMVNWLCENRVFTEDKWK
LVDFNQYTLLSSSHSGSFQIQSALKLAIYKATNLVPTSTFLLHTDFEQVMCIKDNKIVKLL
LCQNYDPLLVGQHVLKWIDKKCGKKNTLWFYGPPSTGKTNLAMAIAKSVPVYGMVN
WNNENFPFNDVAGKSLVVWDEGIIKSTIVEAAKAILGGQPTRVDQKMRGSVAVPGVPV
VITSNGDITFVVSGNTTTTVHAKALKERMVKLNFTVRCSPDMGLLTEADVQQWLTWCN
AQSWDHYENWAINYTFDFPGINADALHPDLQTTPIVTDTSISSSGGESSEELSESSFFNLIT
PGAWNTETPRSSTPIPGTSSGESFVGSPVSSEVVAASWEEAFYTPLADQFRELLVGVDYV
WDGVRGLPVCCVQHINNSGGGLGLCPHCINVGAWYNGWKFREFTPDLVRCSCHVGAS
NPFSVLTCKKCAYLSGLQSFVDYE
SEQ ID NO: 14 Primate Erythroparvovirus 2 VP1 amino acid sequence
(NCBI Ref: YP_009507369.1)
MSEPASKKQKWWDEENAYSDAWLSEFKDIIKDAISLIGEEEEVPVLALKFLQSHLKLDL
KYGVDALSGSDALRFLTEHALPLNAVNKDTKGDSTLGIYLKQHLQDYIDNPDKYTLDLS
HGPLPDFRETEAEHKSFNEPRGDDAVLTKEDLHEGGGVSLTLPFSNYIGPGNQLQAGNP
QSVVDAAARIHDFRYSELIKLGINPYTHWSVADDELLHNIKNEEGFQAQVVRDFFTLKG
LFTSTAHFKGELPAVPEYSASENYPNMASVTSTEGTTGAGGGGSNPVHGVWREGAVFS
DSSVTCTFSRVFVVPYTAEHAYRVFSPPAENCHSAATGESKVCAVSPVMAYATPWHYID
VNCASLYFSPLEFQRLLENYGSIKPSSMSVTLSEVCIKDVTDKPGGGVQVTDSTTGKLCF
LVDDEYQFPYVLGQGQDTLAPELPIWTYLLPQYAYLTVGEVNTKGLTSSTRKQPSEESA
FYVLEHANCLLLGTGSSISTAYTFPPLTAESLEGASQHFYEMYNPLYSSRLAVPSALGGQ
PKVRFVQPTDHAIQPQNFMPGPLVNTVTTAEGDSSSTGAAKALTGISTGSSQNTRISFRP
GPRSQPYHYYDEINQKYINGIDSISYGVTTFGNTAKPQEASQAVGRYPNDKEQSKQLQG
LDIKTFYSNKGDQKYTEEINRPLMVGSIWNRRAFHYETQLWTKLPNLDEGFKTEFSALG
GWALPKPPPMIFLKMQPAPGPEGFASITNSTLAQYATGVLTVTLTFALGPRKHTGRWNP
QPACIPPHAAGHLPYILYDTEVTKNSQNHRHGYEKPEECWSAKKRVHPL
SEQ ID NO: 15 Primate Erythroparvovirus 2 VP2 amino acid sequence
(NCBI Ref: YP_009507370.1)
MASVTSTEGTTGAGGGGSNPVHGVWREGAVFSDSSVTCTFSRVFVVPYTAEHAYRVFS
PPAENCHSAATGESKVCAVSPVMAYATPWHYIDVNCASLYFSPLEFQRLLENYGSIKPSS
MSVTLSEVCIKDVTDKPGGGVQVTDSTTGKLCFLVDDEYQFPYVLGQGQDTLAPELPIW
TYLLPQYAYLTVGEVNTKGLTSSTRKQPSEESAFYVLEHANCLLLGTGSSISTAYTFPPLT
AESLEGASQHFYEMYNPLYSSRLAVPSALGGQPKVRFVQPTDHAIQPQNFMPGPLVNTV
TTAEGDSSSTGAAKALTGISTGSSQNTRISFRPGPRSQPYHYYDEINQKYINGIDSISYGVT
TFGNTAKPQEASQAVGRYPNDKEQSKQLQGLDIKTFYSNKGDQKYTEEINRPLMVGSIW
NRRAFHYETQLWTKLPNLDEGFKTEFSALGGWALPKPPPMIFLKMQPAPGPEGFASITN
STLAQYATGVLTVTLTFALGPRKHTGRWNPQPACIPPHAAGHLPYILYDTEVTKNSQNH
RHGYEKPEECWSAKKRVHPL
SEQ ID NO: 16 Primate Erythroparvovirus 2 NS1 amino acid sequence
(NCBI Ref: YP_009507368.1)
MEMYRGVIQVNANFTDFANDNWWCCFFQLDVDDWPELRGPERLMAHYICKVAALLD
TPSGPFLGCKYFLQVEGNHFDNGFHIHVVIGGPFLTPRNVCSAVEGGFNKVLADFTSPTI
TVQFKPAVSKKGKYHRDGFDFVTYYLMPKLYPNVIYSVTNLEEYQYVCNSLCYRRTMH
KRQQPCNGGSVEQSSVSLYSDGEPANKKSKVVTVRGEKFCSLVDSLIERNIFNENKWKE
TDFKEYAALSASVAGVHQIKTALTLAVSKCNSPAYLGEILTRPNTINFNIRENRIANIFLSN
NYCPLYAGKMFLAWVQKQLGKRNTIWLFGPPSTGKTNIAMSLASAVPTYGMVNWNNE
NFPFNDVPYKSIILWDEGLIKSTVVEAAKSILGGQPCRVDQKNKGSVEVSGTPVLITSNSD
MTRVVCGNTVTLVHQRALKDRMVRFDLTVRCSNALGLIPADEAKQWLWWAQNNACD
AFTQWHLSSDHVAWKVDRTTLCHDFQSEPEPDSELPSSGESVESFDRSDLSTSWLDVQD
QSSSPENSDVEWDIADLLSNEHWIDDLQEDSCSPPRCSTPVAVAEPVEVPTGTGGGLKW
EKNYSVHDTNELRWPMFSVDWVWGTNVKRPVCCLEHDKEFGVHCSLCLSLEVLPMLI
EKSILVPDTLRCSAHGDCTNPFDVLTCKKCRDLSGLMSFLEHE

The representative nucleic acid sequences encoding the VP1, VP2, and NS1 proteins of Primate Erythroparvovirus 2 are available at the NCBI website (World Wide Web at ncbi.nlm.nih.gov) under the NCBI reference sequence: NC_038540.1.

Primate Erythroparvovirus 3 VP (Capsid) amino acid sequence
(NCBI Ref: YP_009507372.1)
SEQ ID NO: 17
MTSSPAKKKPRKWWDDEDAYSEAWFAEFKDILNDVVFAATSEDEGLVFVLKLLQQYY
KLDLTHGLDALSMSDAVDFLTNNALGVSSVNKKSDNTSVLGEYLQKQIENYKNNPNKY
TLQLSHGPLPDFRESEAKHESSNEPRGDDAVLSKKDLHEGGGFSVQLPFSHYIGPGNELQ
AGAPESVVDAAARSHDFRYSELIKLGINPYTQWTVADDELLHNIKNEHGFQAQVVRDY
FTLKGLFTSTAHFKGELPAVPQYSSSENYPSMASVTATEGATGSGGGGSNAVQAVWRE
GAIFTDSSVTCTFSRIFVVPYTAEHAYRFFLLLLKNCHSAATGESKVCAVSPVMGYATP
WHYIDVNCASLYFSPLEFQRLIENYGSIKPSSMQVTLSEICIKDVTDKPGGGVQVTDSTT
GRLCYLVDDEYQFPYVIGQGQDTLAPELPIWTYLLPQYAYLTVGEVNTKGITSATRKQP
SEESAFYVLEHANCLLLGTGSSISSSYQFPSVQAESLEGASQHFYEMYNPLYPSRLAVPS
ALGGQPKVRFVQPTDHAIQPQNFMPGPLVNTITTADGDSSNTGAAKHLQAFLQGSSQNT
RISFRPGPRSQPYHYYDEVNQKYVHGIDSISYGMTTYGFTQKPTEGSQAVGRYPNDKEQ
NKQLQGLNIKTYFNNKGDQKYTEEINRPLMVGSIWNRRAFHYETQLWTKLPNLDEGFK
TEFSALGGWALPKPPPMIFLKMNPAPGPEGFASITSSTLAQYATGILTVTLTFALGPRKHT
GRWNPQPACTPPHAAGHLPYVLYDPEVTKNSQNHRHGYEKPEECWSAKKRVHLL
Primate Erythroparvovirus 3 NS amino acid sequence (NCBI Ref:
YP_009507371.1)
SEQ ID NO: 18
MDMFRGVIQLTANITDFANDSWWCSFLQLDSDDWPELRGVERLVAIFICKVAAVLDNP
SGTSLGCKYFLQAEGNHYDAGFHVHIVIGGPFINARNVCNAVETTFNKVLGDLTDPSMS
VQFKPAVSKKGEYYRDGFDFVTNYLMPKLYPNVIYSVTNLEEYQYVCNSLCYRKNMH
KQHMVSTVDASSSSFMNDMYEPATKRSKSCTVKGEKFRNLVDSLIERNIFSESKWKEVD
FNEFARLSASVAGVHQIKTAITLAVSKCNSPDYLFQILTRPSTIHENIKENRIAQIFLNNNY
CPLYAGEVFLFWIQKQLGKRNTVWLYGPPSTGKTNVAMSLASAVPTYGMVNWNNENF
PFNDVPYKSLILWDEGLIKSTVVEAAKSILGGQPCRVDQKNKGSVEVTGTPVLITSNSDM
TRVVWYTVTLVHQRALKDRMVRFDLTVRCSNALGLIPADEAKQWLWWAQSQPCDAF
TQWHQVSEHVAWKADRTGLFHDFSTKPEQESNAKSSGKSNDSFAGSDLANLSWLDVE
DTSSSSESDLSGDIAELVSNDNWLQSGCPPTRCSTPVTVVEPKQVSPGTGGGLTKWEKN
YSVHQENELAWPMFSVDWVWGSHVKRPVCCVEHDKDLVLPHCNLCLSLEVLPMLIEK
SINVPDTLRCSAHGDCTNPFDVLTCKKCRDLSGLMSFLEHDQ

The representative nucleic acid sequences encoding the capsid and NS proteins of Primate Erythroparvovirus 3 are available at the NCBI website (World Wide Web at ncbi.nlm.nih.gov) under the NCBI reference sequence: NC_038541.1.

Primate Erythroparvovirus 4 VP (Capsid) amino acid sequence
(NCBI Ref: YP_009507374.1)
SEQ ID NO: 19
MTDEKPKEKKWWETGDPFREAWYNQFVKIFTDLVGNDLDLAEILWRHYGINLDNPFSN
PAALPDLVNRIKKNLKDNPDIYTDSLSHGALPDFRESKAEHEKSNEPRGADAILTSKDLH
DGGSISLTLPLTHYIGPGNPLQAGSPTDVVDAAARIHDYRYSELIKLGINPYTHWTVADD
ELLHNVQNVGGFEAQVVKDFFTLKGLFTSTAHFKGELPPVPSYSATEQYPNMATVTATE
GTSGSGGGGSNPVHGVWREGAVFSEDSVSCTFSRVFVVPYAAEHSYRVFSPPAENCHSA
AAGESRVCAVSPVMGYATPWHYIDVNCASLYFSPLEFQRLLENYGSIKPSSMSVTLSEIC
VKDVTDKPGGGVQVTDSTTGRLCFLVDDTYQYPYVLGQGQDTLAPELPIWTYLLPQYA
YLTVGDVNTKGITSSSRKQPTEETAFYVLEHSSCMLLGTGSSISTSYAFPELPYESLEGAA
QHFYEMYNPLYSSRLAVPSALGGQPKVRFVQPTDHALQPQNFMPGPMVNTVTTKEGDS
SNTGAAKALTGFSTGTSQNTRISFRPGPNSQPYHYYDEAEQKYVNSIDSISHGVTTFGDR
QKPNEASESVGRYPNDKEQQKQLQALNIKTYYSNKGDQKYTEEINRPLMVGAVWNRRS
FHYETQLWTKLPNLDENFMAEFSALGGWALKTPPPMIFLKMQPAPGPEGFSGITNTTLA
QYATGTLTVTLTFSLGPRKHTGRWNPQPAVYPPHAAGHLPYVLYDPEVTKTSQTHRHG
YEKPEELWSAKKRVHPL
Primate Erythroparvovirus 4 NS amino acid sequence (NCBI Ref:
YP_009507373.1)
SEQ ID NO: 20
MEMFRGVVHVSANFINFVNDNWWCCFYQLEEDDWPRLQGWERLIAHLIVKVAGEFAV
PGGSTLGLQYFLQAEHNHFDEGFHVHVVVGGPFVTPRNVCNIVETGENKVLRELTEPTY
EVSFKPAISKKGKYARDGFDFVTNYLMPKLYPNVVYSVTNFSEYEYVCNSLAYRRNMH
KKALTNTADEGEGTSTNSEWGPEPKKQKTGTVRGEKFVSLVDSLIERGIFTENKWKQVD
WLKEYACLSGSVAGVHQIKTALTLAISKCNSPEYLCELLTRPSTINFNIKENRICKIFLQN
DYDPLYAGKVFLAWLGKELGKRNTIWLFGPPTTGKTNIAMSLATAVPSYGMVNWNNE
NFPFNDVPHKSIILWDEGLIKSTVVEAAKAILGGQNCRVDQKNKGSVEVQGTPVLITSNN
DMTRVVSGNTVTLIHQRALKDRMVEFDLTVRCSNALGLIPAEECKQWLFWSQHTPCDV
FSRWKEVCEFVAWKSDRTGICYDFSENEDLPGTQTPLLNSPVTSKTSALKKTIAALATA
AVGTLQTSLTNNNWESSEDSGSPPRSSTPLASPERGEVPPGQQWELNTSVNSVNALNWP
MYTVDWVWGSKAQRPVCCLEHDTESSVHCSLCLSLEVLPMLIENSINQPDVIRCSAHAE
CTNPFDVLTCKKCRELSALWSFVKYD

Representative nucleic acid sequences encoding the capsid and NS proteins of Primate Erythroparvovirus 4 are available at the NCBI website (World Wide Web at ncbi.nlm.nih.gov) under the NCBI reference sequence: NC_038542.1.

Rodent Erythroparvovirus 1 VP1 amino acid sequence (NCBI Ref:
YP_009507377.1)
SEQ ID NO: 21
MPKRKGAGEAFRVLLDELFGGILSVGGDAFDDPVSELAEHLTLSGIGDADTFKKWQEK
DLRHIAQLVAEFETQYNKKELDTLVVDEVKKVANKVVPGLGETGAAVANTAKRLKTD
EDPLSFGAPPLTENAPVPVAEPDVAIVSEPNRDTAAEQLERGLAEPDHGGIHLPADRYLG
PGNPLENGPPVDPVDAVARIHDFRYADLEKQGINPYTTYTIADEELLKNLEHKTGGRAAI
ARAFFNFKKLTFPHAHLQGPLPAVKSWKTEQLGLAGMQQASAVSGAGGDHTPAALWA
QGAKFSGDSVTCFMTRRCYLPFDEDPTYRAIAHSESDRSNFTKIMVNTGTHTVMGYTTP
WHYVDYNNMALFFSPQEFQYLLENYEEIAPKSLTTVLSDLVVKDVSIQDQKTQVTDSGT
GGVAIFADESYTYPYVLGNGQRTLPSDIPIQVYELPKYAYLTCGKRTDVGMKGGSLPTH
DSDFFFLEHAMFKIYKTGDFFVSPYSFPSLRPRSLMGASQHFFMMQNPLYDYGMDVLTE
IGTHGQWSSLDKWEYHGRPQNFFPGPKIPSHVAAEGDRGGKAELQKVATGTSVGDDW
YSRYTFRPMPSCQAYSHADPKDPDSDIPVVSIDAVAAGQQSEKPKPPHAKESKFPYKQG
RLPNDIEMAKQLQGVNDKMYLVQTLAGQNTTPAQIIPLMPGSVWNERALHYESQIWTK
IPNLDKGFMTDHPALGGWGMSTPPPQIFIKMIPTPAPSVEGGGTTSTLHQYAIFNMTVKL
EFTLKKRGLAGRWNPQPPVNPPSAVGHLPYVLYDNGQLTGVSSDVQSQNGYERSDELW
TAKSRVRHL
Rodent Erythroparvovirus 1 VP2 amino acid sequence (NCBI Ref:
YP_009507378.1)
SEQ ID NO: 22
MQQASAVSGAGGDHTPAALWAQGAKFSGDSVTCFMTRRCYLPFDEDPTYRAIAHSESD
RSNFTKIMVNTGTHTVMGYTTPWHYVDYNNMALFFSPQEFQYLLENYEEIAPKSLTTVL
SDLVVKDVSIQDQKTQVTDSGTGGVAIFADESYTYPYVLGNGQRTLPSDIPIQVYELPKY
AYLTCGKRTDVGMKGGSLPTHDSDFFFLEHAMFKIYKTGDFFVSPYSFPSLRPRSLMGA
SQHFFMMQNPLYDYGMDVLTEIGTHGQWSSLDKWEYHGRPQNFFPGPKIPSHVAAEGD
RGGKAELQKVATGTSVGDDWYSRYTFRPMPSCQAYSHADPKDPDSDIPVVSIDAVAAG
QQSEKPKPPHAKESKFPYKQGRLPNDIEMAKQLQGVNDKMYLVQTLAGQNTTPAQIIPL
MPGSVWNERALHYESQIWTKIPNLDKGFMTDHPALGGWGMSTPPPQIFIKMIPTPAPSV
EGGGTTSTLHQYAIFNMTVKLEFTLKKRGLAGRWNPQPPVNPPSAVGHLPYVLYDNGQ
LTGVSSDVQSQNGYERSDELWTAKSRVRHL
Rodent Erythroparvovirus 1 NS1 amino acid sequence (NCBI Ref:
YP_009507375.1)
SEQ ID NO: 23
MAQACLSLSWADCFAAVIKLPCPLEEVLSNSQFWQYYVLCKDPLDWPALQVTELAHG
WEVGAYCAFADALYLYLVGRLADEFSAYLLFFQLEPGVENPHIHVVAQATQLSAFNWR
RILTQACHDMALGFLKPDYLGWAKNCVNIKKDKSGRILRSDWQFVETYLLPKVPLSKV
WYAWTNKPEFEPIALSAAARDRLMRGNALCNQPGPGPSFGDRAEIQGPPIKKTKASDEF
YTLCHWLAQEGILTEPAWRQRDLDGYVRMHTSTQGRQQVVSALAMAKNIILDSIPNSV
FATKAEVVTELCFESNRCVRLLRTQGYDPVQFGCWVLRWLDRKTGKKNTIWFYGVAT
TGKTNLANAIAHSLPCYGCVNWTNENFPFNDAPDKCVLFWDEGRVTAKIVESVKAVLG
GQDIRVDQKCKGSSFLRATPVIITSNGDMTVVRDGNTTTFAHRPAFKDRMVRLNFDVRL
PNDFGLITPTEVREWLRYCKEQGDDYEFPDQMYQFPRDVVSVPAPPALPQPGPVTNAPE
EEILDLLTQTNFVTQPGLSIEPAVGPEEEPDVADLGGSPAPAVSSTTESSADEDEDDDTSS
SGDHRGGGGGVMGDLHASSSSFFTSSDSGLPTSVNTSDTPFSFSPVPVHHHGPPTLLPTS
RPTRDLARGRPSFRQYEPLKGRCADSTTFGRPSWAAPCAVYNTAELTRRGAGVRVVKG
SRPGAISGK
Rodent Erythroparvovirus 1 NS2 amino acid sequence (NCBI Ref:
YP_009507376.1)
SEQ ID NO: 24
MPRKKRSLISLPKQTSSLNLGSLLSRPLDLKKNLMSQILEGLQHQQSAAPQSPVPTRTRTT
TPPPLATTEEEEEGSWEIYTLLLPPSLLPVTQDSPLPSTPATPLSPSAPYQCTTTDPQRFSRP
HARHAIWPVGARLSASTSH

Representative nucleic acid sequences encoding the VP1, VP2, NS1 and NS2 proteins of Rodent Erythroparvovirus 1 are available at the NCBI website (World Wide Web at nebi.nlm.nih.gov) under the NCBI reference sequence: NC_038543.1.

Ungulate Erythroparvovirus 1 VP (Capsid) amino acid sequence
(NCBI Ref: YP_009465714.1)
SEQ ID NO: 25
MAFNPLTMSSRLLVPVTPVSKLDLLKKKWFAFPDVSKILLEALSHSGFGDPKKWKEAD
ADIIEALLDEALRLGPRLEKPAWFYDLQRAIGLARFSASLEQTVFLNEMLIKLTRGPVVP
KYPEPDIVIRDPAPLTPEVEAPTSTPENSPDQSSVASDPVEMEEGSSTPIPDPVPQSEDMET
EETTIPDQPPPPSPQIVDEVEDMAMGVEDLSIVEDASEQHQSPAGEPTPDITSSVGNRDDE
SREESREADLQDLSAGLGAAGGSAIAALGSGLIPAATVATAYPRPDQFLRDYLARYDQM
YPSGSRYPPRWEQLKSLYDKGMTVKEVWDLLNKNSNNSNLQAKDTDKKQTAPSSSSAP
QESAAAMASGDKSGVNPSGGSAPLSATVWASGAQFEADHVITHMSRTVFIPFQQAHRY
EPIVWRGRRTADGWLSFWPDHPVIGYKTPWFYLDVNAINRHFSPGEWQEVLERYGSIVP
ESMEIILSDFCIKDVSVVDGKTTVTDSSTGGVCVFVDDGYKFPYVLGHSQNTLPGPLPTD
IYSPPQYAYLTTGKKTKVAAYASGEGPMPMDSIAIPSQETAFYVLENSFYTIQRAGGGFA
HSYNFPSLKPISLEGFSQHWMLMDNPLYPSRLWVPEKVGGASKWGAVKNDDYGKKPL
NWMPGPNIPSHTIEQSDQAGQRVELDRDVEGQKVWTGTSFGSRPENRWSMRPLGVNQP
YAYDAYEDETDKIVTVDAIGYGTAKASAALGQDTGEVPENASVGRVPDDTECNKQGG
GGNHLFQVKSLAHNNFTEQMKNQTVPLMPGSVWQNRALHYESQIWAKIPNVDGEFMC
ERPALGGWGMHDPPPQIFMKMQPVPAPKSLNSTTEAGFPSEHYLHQYAYCVMTVRMR
WKTTTRTGPTRWNPQPTFGPPEATDHIPYILYDRLSTIHKTRGQFTNAYYEEPESVWTAR
GRVRHL
Ungulate Erythroparvovirus 1 NS amino acid sequence (NCBI Ref:
YP_009465713.1)
SEQ ID NO: 26
MESYSRAVIRLPWENIYEAIQEAAWPSLAAVEPQRPGDLPYDWPLLYDEDRRYVVACD
ALWSILQRRAAVFGRWAGYLQLEPSQAGGPGRHLHLLLSAPGIRGRSWTAFLRNAVAE
WARTTVHLNYVDAIDIPRNTHGRILEADADFVFRYLAPKLPLREVTWAWTNEDQFKPF
ALCEPKRRELIQRATTQDRANGLDGPPAKRSRAADEFHQLVHFLADKGIVDPDKWMAL
FPDSYITWSSSAQGRQQVNSACELALQIILTRGVLSRFLAPNPSNIFPENNRAVELLRMQG
HDPVSFGQLVLAWADKQLGKRNTLWFWGPPSTGKTNLALAIARALPRFGMVNWTNEN
FPFNDAPHKCVLVWDEGRITAKIVEAVKSILGGQAVRVDQKCKGSVSLSPTPVLITSNAD
IRYVRDGNIVTGDHVKALSERMVIVHFSTPCPANFGLLKAEEIVDWLNYVKSCPGSITAD
TVQATWGTRSAPNLFEIKRKAPQTASPLGPQAEEQEEAAAYRCPSSPASSRSSSPDIFGIT
KSPAPLEDLSSDSSSECSLPFTPSNAAWFTPMPPARPLQPPLFGVDWIYSTQWKQPVCCL
DHETEPCNLCIDIAERCVLFRVSEPDLLRCPDHRHEENPFDVLLCRHCQALSGLETLQSA

Representative nucleic acid sequences encoding the NS and VP proteins of Ungulate Erythroparvovirus 1 are available at the NCBI website (World Wide Web at ncbi.nlm.nih.gov) under the NCBI reference sequence: NC_037053.1.

### Representative GSH Sequences

PAX5 Genomic safe harbor sequence
>NG_033894.1:184716-186382 Homo sapiens paired box 5 (PAX5),
RefSeqGene (LRG_1384) on chromosome 9
SEQ ID NO: 27
CCCAGCAATGGATCGATGCACGGCTGTCGGGGCCGACAGGCTGACCTTTACTGAGC
TCAGGTTTTCATCTCCCTGTTGGGAGCCCAGGAAGGTCTTGCTGTGGAGAGAGGAAC
GGTGAGAAGCCTTGGCCTGCGAGGGGGAGAGGCTTGGCGTGGGTGCAGTGAAGACA
GCTTCTGAGAGCTGAAAGCCCTTGGAGGTCACTTATTTCAATTTCTTCCAAACACAC
CACTTTTACAGATGAGAAAACTGAGACTTGGTGAGAAATGACTTGTCCAAGGTCACT
CTAAGAGGCTTTGACACAGCTCCAGAATCCAGTGTGTGTGTATGTGTGTGTGTACAC
ATCCAACATACATACATCTGTATATTATAAATATTATACATATTATTACGTATACATA
CACAGATACATTATATGCATGTGTACGTGTATATCGGGAGTGTCTATACATGTGTAT
TATGAAAGCCTGGCTGTGGCTACGTGTGATGCCGTGCCTGCGCTCACTCTGGTCGTC
AACAGTTTGGTCCCGCAACATCCCGGGTAGCCGCCGATCCCTGAGCCACCAGGCATT
TCATGCAGTTCTGCAAAGCCATGGAGAGGAGCTGAAGAAACCTCATGGTCCTTTTCA
AATCGTTTCTTCCTCCTCCTCCTCTGCAAAGATTTTCTCTAAGCCCAGTTTGAATCCTT
CAGAAACAGAACTTGGCTGCGAAGTCACTTTGAAAGACTTTCCATATGTTAATTGCA
GCCGGCCAAGGTCTGGAGCAGAGGTGGGAGCCCACCATCTGCAGACGGGGTCGGCC
CCCAGTGCGCTCTGCAAATCCCCGTCATCTGGCAGGTGTCGTTTTGGGTTAATTAAG
AGCTATACTGAGCCCGTTTACCTGTCACTTCTGAGAATTTTAGGAAACTTTGACTTTC
TTGCCATCTCTGAGCTTTGAGCGAAGGGGAAGCTGAAAACACCTCTGAATCTGGTGA
TGTTTCTGCCTCTGGGATCTCCAGGACAGCTGCATTAAGTGCATCTTATCATAACCCC
TTTTTAAACTTTTTATTTTAATCAGTGTTCTCTAGTTAGTGCATTGGTTTTTACAGTCA
CGTCTTCTATATTGGAAGACAGTACTGTTTGGGGGAAACCCACCATTTGTCTGAAAT
TTCTTAAGGCTCTGCTTTCTCTCTGTGTCTTTGAGGAAACAGCATACATTCCTCTAGC
TTTGTTCTGTGTAATGGCTTTGGAGAAACTTTGAATTTGCAGGTCAGGGGCTCTTTCA
CCCATTGGGGTTTGGGGCTGTCAGTGCTAACCTCAGAGCTCTATGTTCATGGAGGGA
TGACTCAGTTACATCCCCAGATAGCTGGGTTCTCGGTTGGTCAATAGGCCCCCTTCTT
CAGTATGAGAGAATTTTCTCTCTGTGCTGTTGACAATGTTCTATTAATATATCTTGGT
AGGGGTTTGGGTCACACAGATCTATGCATTTGTCAAAACACAGCAATAGCACATTTA
AGATTTGTGTGTTTCATTATGTGTAAATTTTGTATCCAAAGAAAAAACTAGTAAACA
AGTAATGAACTTCAGTTAATTGTATGCATGCTGAAGTACTTAGGGGAAAGTGTACTG
ATGTTTGCATTTACTTGGAAATGAAATACACATTAAGGTGAAAGAAAGGCTAGAGG
GATGAAG
KIF6 Genomic Safe Harbor Sequence
>NG_054928.1:303712-305348 Homo sapiens kinesin family member 6
(KIF6), RefSeqGene on chromosome 6
SEQ ID NO: 28
AGTGGTGTGATCATGGCTTACTGCAGCCTCAACCTCCCAGATTCAAGTGATCCTCCT
GCCTCAGCCTACCGAGTAGCTGGGACTACAGATGCATGCCATCACGCCTGACTAATT
TTACCTTTTGTAGAGATGAGGTCCCTCTGTGTTGCCAAAGGTGGTCTAGAACTCCTG
GGCTCAAATGATCCTCCCCCCTCCCTGGGCCTTCCAAAGTACTGGGATTACAGGTGT
AAGCCAATGCACTCAGCCCCATGTTACTTAATAGAAAGGTTTTTCTTCCCCTTTTTCC
TGCACCCTTTGCTGCTCTCACGGGGAATTTCTAGCATCTCTAAGCTCTGGTCTCCAGT
CTGAGGAAGTTGTGCTGCCTGTATGTGACAAGAGAAATAAGATGTTGGCACATGAA
TAGGATGTTCGCCCTTTGGTGAACTAGAGCATGTGAGCCAATTCTTAAGCCAGATTT
TTCAGCAGAGAACAATTGCAATTCACAATCACATTTTCCAGGCATGACTCATCCCTA
TAGTATACAATAATATGAAGAGAGGCTGGAAACCCCATGCTTGGCAAATACCAGTG
CCCAGGCACTGCAAGCTTTCTTTTGTGGCAGATTTTTCATACAAACTGAGTCCATCA
GTCTCAGAGTCCCATTCAATAACAAAAGAAGAAATAAATGGGGAGATTAACTGCTA
TTGGAAATGAAGGTGTTGAAAATGTAAACTAAACAAAGCAAAGCACCCCTTCACTC
AGTTGGATCCTTCTAACATAGAATCAAACAGCCATCTAAAACCAACAGGAAAACCG
GACCGAGGGTGGAGAGAAACCGTGTGGCACCATCAGGAGGTAACTCCCATGGTGAG
GAGGAGTTTCCTGGCTCCCATTAGACTTTCATTTTAACCCTTATGATTTTTCCTTTTTT
TTTTTTTTTTGTATTTACATCTTTTCTGCTGAACTAAAATGATAAACTCTGAGAGCAG
AGATTAGATTAGAATCTTCTCAGCCTCTCCACAATTTTAAAAGCAGTGCTGGCCACA
GGAAAAAAAAAAAAAGGTACTCAAAAAACACTTTTTTTGTTTGTGAATGACAATTTG
AAATTGACTTTGAGAAATCTTGGCAGCCAAGAAAATGGCTGGAGAAGACTTTACAG
CTTCCGAGAAGTAGGAGGATGCAGCAGGCTTCTGGAGGGTCAGGGGAGGAGCTGAT
CAACTGGAGGCGGGAGAGGGAGGCCATAGTGGGAGAGATGAAACGGGAAAGGAAT
ACTAGCATTTTTTAAAAAGCATAAGGGGAACAAAGGGTGGATCTTTATTACAATAA
AGTGGAGGCAGCCAGGGTACAAGGTACAAGTTTATGGAGGAAAAAAATGGCAAAA
TATAGGCCCAGTCTTCTGTCCTCCTCTCTGACAGGGAAGGGTATTGGATGTTCACTCT
ATGAAAAAGCAACATATTAAGTTAGTTGTTCTAGACAAGAAAAGTAGGAAAGATAT
TGTAGGAACCCTTTGCCCTCAAACACATATTGGCCCACCATTCTCAGAAGGCAATCT
CAGCTGGCATGACAGAGCATCTGGTTGCAGAGGCTCTTGGGGACTGAGTGGCTGCT
GAACGAACACCAGCCCCTCTCTTTGGCCCATGGGTAAAAGCAGCCACTGC

Included above are cDNA, ssDNA, and RNA nucleic acid molecules (e.g., thymidines replaced with uridines), nucleic acid molecules encoding orthologs or variants of the encoded proteins, as well as nucleic acid sequences comprising a nucleic acid sequence having at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of any SEQ ID NO listed above, or a portion thereof. Such nucleic acid molecules can have a function of the full-length nucleic acid as described further herein.

Included above are orthologs or variants of proteins, as well as polypeptide molecules comprising an amino acid sequence having at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed above, or a portion thereof. Such polypeptides can have a function of a full-length polypeptide as described further herein.

### Methods of Preventing or Treating Diseases

In some embodiments, provided herein are methods of preventing or treating a disease using a recombinant virion or pharmaceutical compositions described herein. In some embodiments, recombinant virions disclosed herein provide to the subject a nucleic acid of interest (e.g., those encoding a therapeutic protein or a fragment thereof) transiently, e.g., a nucleic acid transduced by recombinant virions is eventually lost after a certain period of expression. In preferred embodiments, a nucleic acid transduced by recombinant virions integrates stably inside cells.

In some embodiments, provided herein are methods of preventing or treating a disease, comprising administering to a subject in need thereof an effective amount of a recombinant virion or pharmaceutical composition of the present disclosure. In some embodiments, a nucleic acid encodes a protein. In some embodiments, a nucleic acid decreases or eliminates the expression of an endogenous gene. In some embodiments, provided herein are methods of preventing or treating a disease, comprising: (a) administering to a subject in need thereof an effective amount of a recombinant virion described herein comprising a nucleic acid that increases or restores the expression of a gene whose endogenous expression is aberrantly lower than the expression in a healthy subject; or (b) administering to a subject in need thereof an effective amount of a virion described herein comprising a nucleic acid that decreases or eliminates expression of a gene whose endogenous expression is aberrantly higher than expression in a healthy subject.

In some embodiments, provided herein are methods of preventing or treating a disease, comprising: (a) obtaining a plurality of cells from a subject with a disease, (b) transducing cells with a virion described herein, optionally further selecting or screening for transduced cells, and (c) administering an effective amount of transduced cells to a subject. There are advantages of preparing transduced cells in vitro or ex vivo. First, the existence and location of a transgene in the target cell genome can be verified before administering them to a patient, thereby avoiding interfering with cell functions or off target effects. This improves safety, even without use of GSH. Second, transduced cells can be administered to a subject in need thereof without recombinant virions. This eliminates any concern for triggering immune response or inducing neutralizing antibodies that inactivate recombinant virions. Accordingly, transduced cells can be safely redosed or a dose can be titrated without any adverse effect.

In some embodiments, a recombinant virion, pharmaceutical composition, or transduced cells of the present disclosure are administered via intravascular, intracerebral, parenteral, intraperitoneal, intravenous, epidural, intraspinal, intrasternal, intra-articular, intra-synovial, intrathecal, intra-arterial, intracardiac, intramuscular, intranasal, intrapulmonary, skin graft, or oral administration.

In some embodiments, a recombinant virion comprises a nucleic acid that encodes a hemoglobin subunit. In some embodiments, transduced cells are erythroid-lineage cells or bone marrow cells In some embodiments, transduced cells are autologous or allogeneic to a subject.

In some embodiments, diseases includes those described herein, e.g., endothelial dysfunction, cystic fibrosis, cardiovascular disease, diabetes, renal disease, cancer, hemoglobinopathy, anemia, hemophilia, myeloproliferative disorder, coagulopathy, and hemochromatosis. In some embodiments, a disease is selected from sickle cell disease, alpha-thalassemia, beta-thalassemia, hemophilia (e.g. hemophilia A), Fanconi anemia, cystic fibrosis, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), and hematologic cancer.

In some embodiments, provided herein are methods of preventing or treating a hemoglobinopathy, comprising: (a) administering to a subject in need thereof an effective amount of a virion described herein, comprising a nucleic acid that encodes a hemoglobin subunit, or (b) obtaining erythroid-lineage cells or bone marrow cells from a subject in need thereof, transducing cells with a virion described herein, comprising a nucleic acid that encodes a hemoglobin subunit, optionally further selecting or screening for the transduced cells; and administering an effective amount of cells to a subject. In some embodiments, a hemoglobinopathy is beta-thalassemia or sickle cell disease.

In some embodiments, methods of preventing or treating a disease further comprise re-administering at least one additional amount of a virion, pharmaceutical composition, or transduced cells. In some embodiments, re-administering an additional amount is performed after an attenuation in a treatment subsequent to administering an initial effective amount of a virion, pharmaceutical composition, or transduced cells. In some embodiments an additional amount is the same as an initial effective amount. In some embodiments, an additional amount is more than an initial effective amount. In some embodiments, an additional amount is less than an initial effective amount. In certain embodiments, an additional amount is increased or decreased based on expression of an endogenous gene and/or a nucleic acid of a recombinant virion. An endogenous gene includes a biomarker gene whose expression is, e.g., indicative of or relevant to diagnosis and/or prognosis of a disease.

In some embodiments, further provided herein are methods of modulating (i) gene expression, or (ii) function and/or structure of a protein in a cell, the method comprising transducing a cell with a virion or pharmaceutical composition described herein comprising a nucleic acid that modulates gene expression, or function and/or structure of a protein in a cell. In some embodiments, such nucleic acid comprises the sequence encoding CRISPRi or CRISPRa agents. In some embodiments, gene expression, or function and/or structure of a protein is increased or restored. In some embodiments, gene expression, or function and/or structure of a protein is decreased or eliminated.

### Exemplary Diseases

In some embodiments, methods, recombinant virions, and/or pharmaceutical compositions described herein may be used for prevention and/or treatment of various diseases. Recombinant virions and/or pharmaceutical compositions comprising at least one capsid protein of erythroparvovirus have an affinity for hematologic cells, thus rendering them particularly powerful in delivering a nucleic acid (e.g., a therapeutic nucleic acid) to a hematologic cells in vivo (e.g., administering directly to a subject), or in vitro or ex vivo (obtaining a plurality of cells from a subject, transducing said cells using recombinant virions, and administering a subject an effective number of transduced cells). Thus, virion compositions and methods provided herein are particularly effective in preventing or treating a hematologic disease. However, recombinant virions described herein can also bind and transduce certain non-hematologic cells, e.g., endothelial cells, such as myocardial endothelial cells or hepatocytes. Accordingly, the use of recombinant virions is not limited to but extends beyond hematologic diseases.

In some embodiments, in addition to the hematologic diseases described below, recombinant virions described herein can be used for prevention or treatment of a disease such as endothelial dysfunction, cystic fibrosis, cardiovascular disease, peripheral vascular disease, stroke, heart disease (e.g., including congenital heart disease), diabetes, insulin resistance, chronic kidney failure, atherosclerosis, tumor growth (e.g., including those of endothelial cells), metastasis, hypertension (e.g., pulmonary arterial hypertension, other forms of pulmonary hypertension), atherosclerosis, restenosis, Hepatitis C, liver cirrhosis, hyperlipidemia, hypercholesterolemia, metabolic syndrome, renal disease, inflammation, and venous thrombosis.

In some embodiments, a hematologic disease includes any one of the following: hemoglobinopathy (e.g., sickle cell disease, thalassemia, methemoglobinemia), anemia (iron-deficiency anemia, megaloblastic anemia, hemolytic anemias, myelodysplastic syndrome, myelofibrosis, neutropenia, agranulocytosis, Glanzmann's thrombasthenia, thrombocytopenia, Wiskott-Aldrich syndrome, myeloproliferative disorders (e.g., polycythemia vera, erythrocytosis, leukocytosis, thrombocytosis), coagulopathies, a hematologic cancer, hemochromatosis, asplenia, hypersplenism (e.g., Gaucher's disease), hemophagocytic lymphohistiocytosis, tempi syndrome, and AIDS.

In some embodiments, exemplary hemolytic anemia includes: Hereditary spherocytosis, Hereditary elliptocytosis, Congenital dyserythropoietic anemia, Glucose-6-phosphate dehydrogenase deficiency (G6PD), pyruvate kinase deficiency, autoimmune hemolytic anemia (e.g., idiopathic anemia, Systemic lupus erythematosus (SLE), Evans syndrome, Cold agglutinin disease, Paroxysmal cold hemoglobinuria, Infectious mononucleosis), alloimmune hemolytic anemia (e.g., hemolytic disease of the newborn, such as Rh disease, ABO hemolytic disease of the newborn, anti-Kell hemolytic disease of the newborn, Rhesus c hemolytic disease of the newborn, Rhesus E hemolytic disease of the newborn), Paroxysmal nocturnal hemoglobinuria, Microangiopathic hemolytic anemia, Fanconi anemia, Diamond-Blackfan anemia, and Acquired pure red cell aplasia.

In some embodiments, an exemplary coagulopathy includes: thrombocytosis, disseminated intravascular coagulation, hemophilia (e.g., hemophilia A, hemophilia B, hemophilia C), von Willebrand disease, and antiphospholipid syndrome.

In some embodiments, an exemplary hematologic cancer includes: Hodgkin's disease, Non-Hodgkin's lymphoma, Burkitt's lymphoma, Anaplastic large cell lymphoma, Splenic marginal zone lymphoma, T-cell lymphoma (e.g., Hepatosplenic T-cell lymphoma, Angioimmunoblastic T-cell lymphoma, Cutaneous T-cell lymphoma), Multiple myeloma, Waldenström macroglobulinemia, Plasmacytoma, Acute lymphocytic leukemia (ALL), Chronic lymphocytic leukemia (CLL), Acute myelogenous leukemia (AML), Acute megakaryoblastic leukemia, Chronic Idiopathic Myelofibrosis, Chronic myelogenous leukemia (CML), T-cell prolymphocytic leukemia, B-cell prolymphocytic leukemia, Chronic neutrophilic leukemia, Hairy cell leukemia, T-cell large granular lymphocyte leukemia, AIDS-related lymphoma, Sezary syndrome, Waldenström Macroglobulinemia, Chronic Myeloproliferative Neoplasms, Langerhans Cell Histiocytosis, Myelodysplastic Syndromes, and Aggressive NK-cell leukemia.

As used herein, hemoglobinopathy includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

As used herein, thalassemia refers to a hereditary disorder characterized by defective production of hemoglobin. Examples of thalassemias include α- and β-thalassemia. β-thalassemias are caused by a mutation in the beta globin chain, and can occur in a major or minor form. In the major form of β-thalassemia, children are normal at birth, but develop anemia during the first year of life. A mild form of β-thalassemia produces small red blood cells and the thalassemias are caused by deletion of a gene or genes from the globin chain, α-thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles. Hb Bart syndrome, the most severe form of a thalassemia, results from the loss of all four α-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. Substitution of Hb Bart or HbH for normal hemoglobin causes anemia and the other serious health problems associated with a thalassemia.

As used herein, sickle cell disease refers to a group of autosomal recessive genetic blood disorders, which results from mutations in a globin gene and which is characterized by red blood cells that under hypoxic conditions, convert from the typical biconcave form into an abnormal, rigid, sickle shape that cannot course through capillaries, thereby exacerbating the hypoxia. They are defined by the presence of βs-gene coding for a β-globin chain variant in which glutamic acid is substituted by valine at amino acid position 6 of the peptide, and second β-gene that has a mutation mat allows for the crystallization of HbS leading to a clinical phenotype. Sickle cell anemia refers to a specific form of sickle cell disease in patients who are homozygous for the mutation that causes HbS. Other common forms of sickle cell disease include HbS/β-thalassemia, HbS/HbC and HbS/HbD.

In certain embodiments, methods and compositions are provided herein to treat, prevent, or ameliorate a hemoglobinopathy that is selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In some embodiments, a hemoglobinopathy is β-thalassemia. In some embodiments, the hemoglobinopathy is sickle cell anemia. In various embodiments, recombinant virions described herein are administered in vivo by direct injection to a cell, tissue, or organ of a subject in need of gene therapy. In various other embodiments, cells are transduced in vitro or ex vivo with recombinant virions described herein. Transduced cells are then administered to a subject in need of gene therapy, e.g., within a pharmaceutical formulation disclosed herein.

As described above, provided herein are methods of preventing or treating a hemoglobinopathy in a subject. In various embodiments, the method comprises administering an effective amount of a cell transduced with recombinant virions described herein or a population of the said transduced cells (e.g., HSCs, CD34+ or CD36 cells, erythroid lineage cells, embryonic stem cells, or iPSCs) to the subject. For prevention or treatment, the amount administered can be an amount effective in producing the desired clinical benefit. An effective amount can be provided in one or a series of administrations. An effective amount can be provided in a bolus or by continuous perfusion. An effective amount can be administered to a subject in one or more doses. In terms of prevention or treatment, an effective amount is an amount that is sufficient to palliate, ameliorate, stabilize, reverse or slow the progression of a disease, or otherwise reduce the pathological consequences of a disease. The effective amount is generally determined by a physician on a case-by-case basis and is within the ordinary skill of one in the art. Several factors are typically taken into account when determining an appropriate dosage to achieve an effective amount. These factors include age, sex and weight of the subject, the condition being prevented or treated, the severity of the condition.

In some embodiments, a disease prevented or treated includes one selected from those presented in Table 3.

TABLE 3

| | | | | |
|---|---|---|---|---|
| | Defective | Materials | Major Organ | CNS |
| Disease | Protein | Stored | Systems Affected | Pathology |

Sphingolipidoses

| | | | | |
|---|---|---|---|---|
| Fabry | α-Galactosidase A | Globotriasylceramide | Kidney, heart | + |
| | | and blood Group B | | |
| | | substances | | |
| Gaucher | β-Glucosidase | Glucosylceramide | Spleen, liver, | + (types |
| | | glucosylsphingosine | bone marrow | 2 and 3) |
| Nieman-Pick A | Sphingomyclinase | Sphingomyclin | Spleen, liver, bone | + (type A) |
| and B | | | marrow, lung (type B) | |
| GM1 Gangliosidosis | β-Galactosidase | GM1 ganglioside | Skeleton, heart | + |

Mucopolysaccharidoses

| | | | | |
|---|---|---|---|---|
| MPS I (Hurler, Scheie, | α-Iduronidase | Dermatan sulphate and | Organomegaly, | + |
| Hurler/Scheie) | | heparan sulphate | skeleton, eye | |
| MPS II (Hunter) | Iduronate-2-sulphatase | Dermatan sulphate and | Organomegaly, heart | + |
| | | heparan sulphate | | |
| MPS VI (Maroteaux- | acetylgalaetosamine-4- | Dermatan sulphate | Spleen, liver, heart, | − |
| L [symbol] y) | sulphatase (arylsulpha- | | macrocephaly, | |
| | tase B) | | | |

[symbol]

indicates data missing or illegible when filed

In some embodiments, following administration of one or more of the presently disclosed transduced cells, peripheral blood of the subject is collected and hemoglobin level is measured. A therapeutically relevant level of hemoglobin is produced following administration of recombinant virions or cells transduced with recombinant virions. Therapeutically relevant level of hemoglobin is a level of hemoglobin that is sufficient (1) to improve anemia, (2) to improve or restore the ability of a subject to produce red blood cells containing normal hemoglobin, (3) to improve or correct ineffective erythropoiesis in the subject, (4) to improve or correct extra-medullary hematopoiesis (e.g., splenic and hepatic extra-medullary hematopoiesis), and/or(S) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dl, Hb, from about 11.5 g/dl. Hb to about 12 g/dl, Hb, from about 12 g/dl. Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb. In certain embodiments, the therapeutically relevant level of hemoglobin is maintained in the subject for at least 3 days, for at least 1 week, for at least 2 weeks, for at least 1 month, for at least 2 months, for at least 4 months, for at least about 6 months, for at least about 12 months (or 1 year), for at least about 24 months (or 2 years). In certain embodiments, the therapeutically relevant level of hemoglobin is maintained in the subject for up to about 6 months, for up to about 12 months (or 1 year), for up to about 24 months (or 2 years). In certain embodiments, the therapeutically relevant level of hemoglobin is maintained in the subject for about 3 days, for about 1 week, for about 2 weeks, for about 1 month, for about 2 months, for about 4 months, for about 6 months, for about 12 months (or 1 year), for about 24 months (or 2 years). In certain embodiments, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain embodiments, a transduced cell is autologous to a subject being administered with a cell. In some embodiments, a transduced cell is from bone marrow or mobilized cells in peripheral circulation, autologous to a subject being administered with a cell. In some embodiments, a transduced cell is allogeneic to a subject being administered with a cell. In some embodiments, a transduced cell is from bone marrow autologous to a subject being administered with a cell.

The present disclosure also provides a method of increasing the proportion of red blood cells or erythrocytes compared to white blood cells or leukocytes in a subject. In various embodiments, the method comprises administering an effective amount of recombinant virions described herein or cells transduced with recombinant virions (e.g., HSCs, CD34+ or CD36 cells, erythroid lineage cells, embryonic stem cells, or iPSCs) to a subject, wherein the proportion of red blood cell progeny cells of the hematopoietic stem cells are increased compared to white blood cell progeny cells of the hematopoietic stem cells in a subject.

The quantity of transduced cells to be administered will vary for the subject and/or the disease being prevented or treated. In some embodiments, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed transduced cells are administered to a subject. Depending on the needs, the subject may need multiple doses of the transduced cells. The precise determination of what would be considered an effective dose may be based on factors individual to each subject, including their size, age, sex, weight, and condition of the particular subject. Dosages can be readily ascertained by those skilled in the art from this disclosure and the knowledge in the art.

Without being bound to any particular theory, an important advantage provided by compositions and methods described herein is an efficient way of treating a subject afflicted with any disease (e.g., a hemoglobinopathy) or preventing any disease in a subject, e.g., those at risk of developing such disease. Such at risk subjects can be identified by certain genetic mutations they carry, and/or environmental or physical factors (e.g., sex, age of the subject). The highly efficient and safe gene therapy is achieved by using the compositions and methods described herein (e.g., recombinant virions comprising at least one capsid protein of an erythroparvovirus). For example, the targeted integration of a nucleic acid (e.g., therapeutic nucleic acid) to a GSH reduces the chances of deleterious mutation, transformation, or oncogene activation of cellular genes in transduced cells. In addition, the specific tropism of a recombinant virion allows targeting to a specific cell type.

### Hemophilia A

Hemophilia A is an inherited bleeding disorder in which the blood does not clot normally. People with hemophilia A bleed more than normal after an injury, surgery, or dental procedure. This disorder can be severe, moderate, or mild. In severe cases, heavy bleeding occurs after minor injury or even when there is no injury (spontaneous bleeding). Bleeding into the joints, muscles, brain, or organs can cause pain and other serious complications. In milder forms, there is no spontaneous bleeding, and the disorder might only be diagnosed after a surgery or serious injury. Hemophilia A is caused by having low levels of a protein called factor VIII. Factor VIII is needed to form blood clots. The disorder is inherited in an X-linked recessive manner and is caused by changes (mutations) in the F8 gene. The diagnosis of hemophilia A is made through clinical symptoms and specific laboratory tests to measure the amount of clotting factors in the blood. The main prevention or treatment is replacement therapy, during which clotting factor VIII is dripped or injected slowly into a vein. Hemophilia A mainly affects males. With prevention or treatment, most people with this disorder do well. Some people with severe hemophilia A may have a shortened lifespan due to the presence of other health conditions and rare complications of the disorder.

Patients afflicted with hemophilia A stands to benefit from gene therapy that introduces a F8 transgene encoding a full length factor VIII (FVIII) or a B-domain-deleted FVIII (e.g., FVIII-SQ, p-VIII, p-VIII-LMW; Sandberg et al. (2001) Thromb Haemost 85:93-100), which retains activity necessary to provide therapeutic benefits in human (Rangarajan et al. (2017) N Engl J Med 377:2519-30). Recombinant virions, pharmaceutical compositions, and methods of the present disclosure provide improved viral vectors and prevention/treatment methods for patients afflicted with hemophilia A, in part due to the ability of recombinant virions to package larger genes compared with AAV, and low immunogenicity.

### Compositions and Methods for Producing a Recombinant Virion.

In some embodiments, provided herein are methods of producing a recombinant virion described herein. The number of vectors described below may be consolidated by incorporating structural and/or nonstructural genes into one or more vectors. Certain erythroparvovirus genomic sequence may also be integrated into the baculovirus genome to contain structural (e.g., encoding VP protein(s)) and/or nonstructural genes.

In some embodiments, the methods of producing a recombinant virion comprises: (1) providing at least one vector comprising (i) a nucleotide sequence comprising at least one ITR nucleotide sequence, optionally further comprising a heterologous nucleic acid operably linked to a promoter for expression in a target cell, (ii) a nucleotide sequence comprising at least one gene encoding an erythroparvovirus (e.g., B19) VP1 capsid protein and/or VP2 capsid protein operably linked to at least one expression control sequence for expression in a host cell (e.g., an insect cell, e.g., a mammalian cell), and (iii) a nucleotide sequence comprising (A) at least one replication protein of erythroparvovirus (e.g., B19) operably linked to at least one expression control sequence for expression in a host cell, (B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or (C) a combination of (A) and (B), (2) introducing said at least one vector into a host cell, and (3) maintaining said host cell under conditions such that a recombinant virion described herein is produced. In preferred embodiments, the vector is a host cell-compatible vector that comprises a promoter that facilitates the expression of a nucleic acid in host cells.

In some embodiments, two vectors are provided: (a) a first vector comprising a nucleotide sequence comprising at least one ITR nucleotide sequence, optionally further comprising a heterologous nucleic acid operably linked to a promoter for expression in a target cell, and (b) a second vector comprising (i) a nucleotide sequence comprising at least one gene encoding an erythroparvovirus (e.g., B19) VP1 capsid protein and/or VP2 capsid protein operably linked to at least one expression control sequence for expression in a host cell (e.g., an insect cell, e.g., a mammalian cell), and (ii) a nucleotide sequence comprising (A) at least one replication protein of erythroparvovirus (e.g., B19) operably linked to at least one expression control sequence for expression in a host cell, (B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or (C) a combination of (A) and (B).

In some embodiments, three vectors are provided: (a) a first vector comprising a nucleotide sequence comprising at least one ITR nucleotide sequence, optionally further comprising a heterologous nucleic acid operably linked to a promoter for expression in a target cell, (b) a second vector comprising a nucleotide sequence comprising a gene encoding an erythroparvovirus (e.g., B19) VP1 capsid protein and/or VP2 capsid protein operably linked to at least one expression control sequence for expression in a host cell (e.g., an insect cell, e.g., a mammalian cell), and (c) a third vector comprising a nucleotide sequence comprising (A) at least one replication protein of erythroparvovirus (e.g., B19) operably linked to at least one expression control sequence for expression in a host cell, (B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or (C) a combination of (A) and (B).

In some embodiments, provided herein are methods of producing a recombinant virion described herein in a host cell (e.g., an insect cell, e.g., a mammalian cell), the method comprising: (1) providing a host cell comprising (i) a nucleotide sequence comprising at least one ITR nucleotide sequence, optionally further comprising a heterologous nucleic acid operably linked to a promoter for expression in a target cell, (ii) a nucleotide sequence comprising at least one gene encoding erythroparvovirus (e.g., B19) VP1 capsid protein and/or VP2 capsid protein operably linked to at least one expression control sequence for expression in a host cell, and (iii) a nucleotide sequence comprising (A) at least one replication protein of erythroparvovirus (e.g., B19) operably linked to at least one expression control sequence for expression in a host cell, (B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or (C) a combination of (A) and (B), optionally, at least one vector, wherein at least one of (i), (ii), (iii) (A), (iii) (B), and (iii) (C) is/are stably integrated in the host cell genome, and the at least one vector, when present, comprises the remainder of the (i), (ii), (iii) (A), (iii) (B), and (iii) (C) nucleotide sequences which is/are not stably integrated in the host cell genome, and (2) maintaining the host cell under conditions such that a recombinant virion is produced.

In some embodiments, provided herein are methods of producing a recombinant virion having at least one capsid protein of erythroparvovirus (e.g., B19) or a genotypic variant thereof. In some embodiments, the at least one replication protein is a nonstructural protein (e.g., NS, NS1, and/or NS2), of the human erythroparvovirus (e.g., B19) or a genotypic variant thereof.

In some embodiments, provided herein are methods of producing a recombinant virion having at least one capsid protein of an erythroparvovirus, wherein an erythroparvovirus is selected from primate erythroparvovirus 1 (human erythroparvovirus B19), primate erythroparvovirus 4 (pig-tailed macaque parvovirus), primate erythroparvovirus 3 (rhesus macaque parvovirus), primate erythroparvovirus 2 (simian parvovirus), rodent erythroparvovirus 1, ungulate erythroparvovirus 1, and a genotypic variant thereof. In some embodiments, the at least one replication protein is a nonstructural protein (e.g., NS, NS1, and/or NS2) of an erythroparvovirus or a genotypic variant thereof.

In some embodiments, vectors, compositions, recombinant virions, or populations of recombinant virions comprise a nucleotide sequence comprising at least one gene encoding an erythroparvovirus VP1 capsid protein. In some embodiments, an exemplary nucleotide sequence is at least 85%, 90%, 95%, 98% or 99% identical to the sequences described herein. One skilled in the art would recognize that nucleotide sequences may undergo additional modifications including codon-optimization, introduction of novel but functionally equivalent (e.g., silent mutations), addition of reporter sequences, and/or other routine modification.

Among other things, the present disclosure includes exemplary nucleotide sequences encoding an erythroparvovirus VP1 capsid protein described herein as shown in Table 4.

Table 4 shows exemplary nucleotide sequences comprising at least one gene encoding an erythroparvovirus VP1 capsid protein described herein.

TABLE 4

| Exemplary | | SEQ ID |
|---|---|---|
| Construct | Sequence | NO: |
| Exemplary B19 | ATCATGGAGATAATTAAAATGATAACCATCTCGCAA | SEQ ID |
| Construct 1 | ATAAATAAGTATTTTACTGTTTTCGTAACAGTTTTGTA | NO: 29 |
| comprising an | ATAAAAAAACCTATAAATATTCCGGATTATTCATACC | |
| erythroparvovirus | GTCCCACCATCGGGCGCGGATCTGCCACCACGAGTA | |
| B19 VP1 capsid | AAGAAAGTGGCAAGTGGTGGGAAAGTGACGACGAAT | |
| nucleotide | TTGCTAAAGCTGTGTATCAGCAATTTGTGGAATTTTA | |
| sequence | CGAAAAGGTTACTGGAACAGACTTAGAGCTTATTCA | |
| | AATATTAAAAGATCATTATAATATTTCTTTAGATAAT | |
| | CCCCTAGAAAACCCATCCTCTCTGTTTGACTTAGTTG | |
| | CTCGCATTAAAAATAACCTTAAAAATTCTCCAGACTT | |
| | ATATAGTCATCATTTTCAAAGTCACGGACAGTTATCT | |
| | GACCACCCCCACGCCTTATCATCCAGTAGCAGTCACG | |
| | CAGAACCTAGAGGAGAAGACGCAGTATTATCTAGTG | |
| | AAGACTTACACAAGCCTGGGCAAGTTAGCGTACAAC | |
| | TACCCGGTACTAACTACGTTGGGCCTGGCAACGAGCT | |
| | ACAAGCTGGGCCCCCGCAAAGTGCTGTTGACAGTGCT | |
| | GCAAGGATTCACGACTTTAGGTATAGCCAACTCGCTA | |
| | AGCTCGGAATAAATCCATATACTCATTGGACTGTAGC | |
| | AGACGAAGAGCTTTTAAAAAATATAAAAAACGAAAC | |
| | TGGGTTTCAAGCACAAGTAGTAAAAGACTACTTTACT | |
| | TTAAAAGGTGCAGCTGCCCCTGTGGCCCATTTTCAAG | |
| | GAAGTTTGCCGGAAGTTCCCGCTTACAACGCCTCAGA | |
| | AAAATACCCAAGCATGACTTCAGTTAATTCTGCAGAA | |
| | GCCAGCACTGGTGCAGGAGGGGGGGGCAGTAATCCT | |
| | GTCAAAAGCATGTGGAGTGAGGGGGCCACTTTTAGT | |
| | GCCAACTCTGTGACTTGTACATTTTCCAGGCAGTTTTT | |
| | AATTCCATATGACCCAGAGCACCATTATAAGGTGTTT | |
| | TCTCCCGCAGCAAGTAGCTGCCACAATGCCAGTGGA | |
| | AAGGAGGCAAAGGTTTGCACCATTAGTCCCATAATG | |
| | GGATACTCAACCCCATGGAGATATTTAGATTTTAATG | |
| | CTTTAAACTTATTTTTTTCACCTTTAGAGTTTCAGCAC | |
| | TTAATTGAAAATTATGGAAGTATAGCTCCTGATGCTT | |
| | TAACTGTAACCATATCAGAAATTGCTGTTAAGGATGT | |
| | TACAGACAAAACTGGAGGGGGGGTGCAGGTTACTGA | |
| | CAGCACTACAGGGCGCCTATGCATGTTAGTAGACCAT | |
| | GAATACAAGTACCCATATGTGTTAGGGCAAGGTCAA | |
| | GATACTTTAGCCCCAGAACTTCCTATTTGGGTCTACTT | |
| | TCCCCCTCAATATGCTTACTTAACAGTAGGAGATGTT | |
| | AACACACAAGGAATTTCTGGAGACAGCAAAAAATTA | |
| | GCAAGTGAAGAATCAGCATTTTATGTTTTGGAACACA | |
| | GTTCTTTTCAGCTTTTAGGTACAGGAGGTACAGCAAC | |
| | TATGTCTTATAAGTTTCCTCCAGTGCCCCCAGAAAAT | |
| | TTAGAGGGCTGCAGTCAACACTTTTATGAGATGTACA | |
| | ATCCCTTATACGGATCCCGCTTAGGGGTTCCTGACAC | |
| | ATTAGGAGGTGACCCAAAATTTAGATCTTTAACACAT | |
| | GAAGACCATGCAATTCAGCCCCAAAACTTCATGCCA | |
| | GGGCCACTAGTAAACTCAGTGTCTACAAAGGAGGGA | |
| | GACAGCTCTAATACTGGAGCTGGGAAAGCCTTAACA | |
| | GGCCTTAGCACAGGTACCTCTCAAAACACTAGAATAT | |
| | CCTTACGCCCGGGGCCAGTGTCTCAGCCGTACCACCA | |
| | CTGGGACACAGATAAATATGTCACAGGAATAAATGC | |
| | TATTTCTCATGGTCAGACCACTTATGGTAACGCTGAA | |
| | GACAAAGAGTATCAGCAAGGAGTGGGTAGATTTCCA | |
| | AATGAAAAAGAACAGCTAAAACAGTTACAGGGTTTA | |
| | AACATGCACACCTACTTTCCCAATAAAGGAACCCAGC | |
| | AATATACAGATCAAATTGAGCGCCCCCTAATGGTGG | |
| | GTTCTGTATGGAACAGAAGAGCCCTTCACTATGAAAG | |
| | CCAGCTGTGGAGTAAAATTCCAAATTTAGATGACAGT | |
| | TTTAAAACTCAGTTTGCAGCCTTAGGAGGATGGGGTT | |
| | TGCATCAGCCACCTCCTCAAATATTTTTAAAAATATT | |
| | ACCACAAAGTGGGCCAATTGGAGGTATTAAATCAAT | |
| | GGGAATTACTACCTTAGTTCAGTATGCCGTGGGAATT | |
| | ATGACAGTAACCATGACATTTAAATTGGGGCCCCGTA | |
| | AAGCTACGGGACGGTGGAATCCTCAACCTGGAGTAT | |
| | ATCCCCCGCACGCAGCAGGTCATTTACCATATGTACT | |
| | ATATGACCCTACAGCTACAGATGCAAAACAACACCA | |
| | CAGACATGGATATGAAAAGCCTGAAGAATTGTGGAC | |
| | AGCCAAAAGCCGTGTGCACCCATTGTAACTCGAGGC | |
| | ATGCGGTACCAAGCTTGTCGAGAAGTACTAGAGGAT | |
| | CATAATCAGCCATACCACATTTGTAGAGGTTTTACTT | |
| | GCTTTAAAAAACCTCCCACACCTCCCCCTGAACCTGA | |
| | AACATAAAATGAATGCAATTGTTGTTGTTAACTTGTT | |
| | TATTGCAGCTTATAATGGTTACAAATAAAGCAATAGC | |
| | ATCACAAATTTCACAAATAAAGCATTTTTTTCACTGC | |
| | ATTCTAGTTGTGGTTTGTCCAAACTCATCAATGTATCT | |
| | TATCATGTCTGGATC | |
| Exemplary B19 | ATCATGGAGATAATTAAAATGATAACCATCTCGCAA | SEQ ID |
| Construct 2 | ATAAATAAGTATTTTACTGTTTTCGTAACAGTTTTGTA | NO: 30 |
| comprising an | ATAAAAAAACCTATAAATATTCCGGATTATTCATACC | |
| erythroparvovirus | GTCCCACCATCGGGCGCGGATCTGCCACCCTGAGTAA | |
| B19 VP1 capsid | AGAAAGTGGCAAGTGGTGGGAAAGTGACGACGAATT | |
| nucleotide | TGCTAAAGCTGTGTATCAGCAATTTGTGGAATTTTAC | |
| sequence | GAAAAGGTTACTGGAACAGACTTAGAGCTTATTCAA | |
| | ATATTAAAAGATCATTATAATATTTCTTTAGATAATC | |
| | CCCTAGAAAACCCATCCTCTCTGTTTGACTTAGTTGCT | |
| | CGCATTAAAAATAACCTTAAAAATTCTCCAGACTTAT | |
| | ATAGTCATCATTTTCAAAGTCACGGACAGTTATCTGA | |
| | CCACCCCCACGCCTTATCATCCAGTAGCAGTCACGCA | |
| | GAACCTAGAGGAGAAGACGCAGTATTATCTAGTGAA | |
| | GACTTACACAAGCCTGGGCAAGTTAGCGTACAACTA | |
| | CCCGGTACTAACTACGTTGGGCCTGGCAACGAGCTAC | |
| | AAGCTGGGCCCCCGCAAAGTGCTGTTGACAGTGCTGC | |
| | AAGGATTCACGACTTTAGGTATAGCCAACTCGCTAAG | |
| | CTCGGAATAAATCCATATACTCATTGGACTGTAGCAG | |
| | ACGAAGAGCTTTTAAAAAATATAAAAAACGAAACTG | |
| | GGTTTCAAGCACAAGTAGTAAAAGACTACTTTACTTT | |
| | AAAAGGTGCAGCTGCCCCTGTGGCCCATTTTCAAGGA | |
| | AGTTTGCCGGAAGTTCCCGCTTACAACGCCTCAGAAA | |
| | AATACCCAAGCATGACTTCAGTTAATTCTGCAGAAGC | |
| | CAGCACTGGTGCAGGAGGGGGGGGCAGTAATCCTGT | |
| | CAAAAGCATGTGGAGTGAGGGGGCCACTTTTAGTGC | |
| | CAACTCTGTGACTTGTACATTTTCCAGGCAGTTTTTAA | |
| | TTCCATATGACCCAGAGCACCATTATAAGGTGTTTTC | |
| | TCCCGCAGCAAGTAGCTGCCACAATGCCAGTGGAAA | |
| | GGAGGCAAAGGTTTGCACCATTAGTCCCATAATGGG | |
| | ATACTCAACCCCATGGAGATATTTAGATTTTAATGCT | |
| | TTAAACTTATTTTTTTCACCTTTAGAGTTTCAGCACTT | |
| | AATTGAAAATTATGGAAGTATAGCTCCTGATGCTTTA | |
| | ACTGTAACCATATCAGAAATTGCTGTTAAGGATGTTA | |
| | CAGACAAAACTGGAGGGGGGGTGCAGGTTACTGACA | |
| | GCACTACAGGGCGCCTATGCATGTTAGTAGACCATGA | |
| | ATACAAGTACCCATATGTGTTAGGGCAAGGTCAAGA | |
| | TACTTTAGCCCCAGAACTTCCTATTTGGGTCTACTTTC | |
| | CCCCTCAATATGCTTACTTAACAGTAGGAGATGTTAA | |
| | CACACAAGGAATTTCTGGAGACAGCAAAAAATTAGC | |
| | AAGTGAAGAATCAGCATTTTATGTTTTGGAACACAGT | |
| | TCTTTTCAGCTTTTAGGTACAGGAGGTACAGCAACTA | |
| | TGTCTTATAAGTTTCCTCCAGTGCCCCCAGAAAATTT | |
| | AGAGGGCTGCAGTCAACACTTTTATGAGATGTACAAT | |
| | CCCTTATACGGATCCCGCTTAGGGGTTCCTGACACAT | |
| | TAGGAGGTGACCCAAAATTTAGATCTTTAACACATGA | |
| | AGACCATGCAATTCAGCCCCAAAACTTCATGCCAGG | |
| | GCCACTAGTAAACTCAGTGTCTACAAAGGAGGGAGA | |
| | CAGCTCTAATACTGGAGCTGGGAAAGCCTTAACAGG | |
| | CCTTAGCACAGGTACCTCTCAAAACACTAGAATATCC | |
| | TTACGCCCGGGGCCAGTGTCTCAGCCGTACCACCACT | |
| | GGGACACAGATAAATATGTCACAGGAATAAATGCTA | |
| | TTTCTCATGGTCAGACCACTTATGGTAACGCTGAAGA | |
| | CAAAGAGTATCAGCAAGGAGTGGGTAGATTTCCAAA | |
| | TGAAAAAGAACAGCTAAAACAGTTACAGGGTTTAAA | |
| | CATGCACACCTACTTTCCCAATAAAGGAACCCAGCAA | |
| | TATACAGATCAAATTGAGCGCCCCCTAATGGTGGGTT | |
| | CTGTATGGAACAGAAGAGCCCTTCACTATGAAAGCC | |
| | AGCTGTGGAGTAAAATTCCAAATTTAGATGACAGTTT | |
| | TAAAACTCAGTTTGCAGCCTTAGGAGGATGGGGTTTG | |
| | CATCAGCCACCTCCTCAAATATTTTTAAAAATATTAC | |
| | CACAAAGTGGGCCAATTGGAGGTATTAAATCAATGG | |
| | GAATTACTACCTTAGTTCAGTATGCCGTGGGAATTAT | |
| | GACAGTAACCATGACATTTAAATTGGGGCCCCGTAA | |
| | AGCTACGGGACGGTGGAATCCTCAACCTGGAGTATA | |
| | TCCCCCGCACGCAGCAGGTCATTTACCATATGTACTA | |
| | TATGACCCTACAGCTACAGATGCAAAACAACACCAC | |
| | AGACATGGATATGAAAAGCCTGAAGAATTGTGGACA | |
| | GCCAAAAGCCGTGTGCACCCATTGTAACTCGAGGCAT | |
| | GCGGTACCAAGCTTGTCGAGAAGTACTAGAGGATCA | |
| | TAATCAGCCATACCACATTTGTAGAGGTTTTACTTGC | |
| | TTTAAAAAACCTCCCACACCTCCCCCTGAACCTGAAA | |
| | CATAAAATGAATGCAATTGTTGTTGTTAACTTGTTTA | |
| | TTGCAGCTTATAATGGTTACAAATAAAGCAATAGCAT | |
| | CACAAATTTCACAAATAAAGCATTTTTTTCACTGCAT | |
| | TCTAGTTGTGGTTTGTCCAAACTCATCAATGTATCTTA | |
| | TCATGTCTGGATC | |
| Exemplary B19 | ATCATGGAGATAATTAAAATGATAACCATCTCGCAA | SEQ ID |
| Construct 3 | ATAAATAAGTATTTTACTGTTTTCGTAACAGTTTTGTA | NO: 31 |
| comprising an | ATAAAAAAACCTATAAATATTCCGGATTATTCATACC | |
| erythroparvovirus | GTCCCACCATCGGGCGCGGATCTGCCACCTTGAGTAA | |
| B19 VP1 capsid | AGAAAGTGGCAAGTGGTGGGAAAGTGACGACGAATT | |
| nucleotide | TGCTAAAGCTGTGTATCAGCAATTTGTGGAATTTTAC | |
| sequence | GAAAAGGTTACTGGAACAGACTTAGAGCTTATTCAA | |
| | ATATTAAAAGATCATTATAATATTTCTTTAGATAATC | |
| | CCCTAGAAAACCCATCCTCTCTGTTTGACTTAGTTGCT | |
| | CGCATTAAAAATAACCTTAAAAATTCTCCAGACTTAT | |
| | ATAGTCATCATTTTCAAAGTCACGGACAGTTATCTGA | |
| | CCACCCCCACGCCTTATCATCCAGTAGCAGTCACGCA | |
| | GAACCTAGAGGAGAAGACGCAGTATTATCTAGTGAA | |
| | GACTTACACAAGCCTGGGCAAGTTAGCGTACAACTA | |
| | CCCGGTACTAACTACGTTGGGCCTGGCAACGAGCTAC | |
| | AAGCTGGGCCCCCGCAAAGTGCTGTTGACAGTGCTGC | |
| | AAGGATTCACGACTTTAGGTATAGCCAACTCGCTAAG | |
| | CTCGGAATAAATCCATATACTCATTGGACTGTAGCAG | |
| | ACGAAGAGCTTTTAAAAAATATAAAAAACGAAACTG | |
| | GGTTTCAAGCACAAGTAGTAAAAGACTACTTTACTTT | |
| | AAAAGGTGCAGCTGCCCCTGTGGCCCATTTTCAAGGA | |
| | AGTTTGCCGGAAGTTCCCGCTTACAACGCCTCAGAAA | |
| | AATACCCAAGCATGACTTCAGTTAATTCTGCAGAAGC | |
| | CAGCACTGGTGCAGGAGGGGGGGGCAGTAATCCTGT | |
| | CAAAAGCATGTGGAGTGAGGGGGCCACTTTTAGTGC | |
| | CAACTCTGTGACTTGTACATTTTCCAGGCAGTTTTTAA | |
| | TTCCATATGACCCAGAGCACCATTATAAGGTGTTTTC | |
| | TCCCGCAGCAAGTAGCTGCCACAATGCCAGTGGAAA | |
| | GGAGGCAAAGGTTTGCACCATTAGTCCCATAATGGG | |
| | ATACTCAACCCCATGGAGATATTTAGATTTTAATGCT | |
| | TTAAACTTATTTTTTTCACCTTTAGAGTTTCAGCACTT | |
| | AATTGAAAATTATGGAAGTATAGCTCCTGATGCTTTA | |
| | ACTGTAACCATATCAGAAATTGCTGTTAAGGATGTTA | |
| | CAGACAAAACTGGAGGGGGGGTGCAGGTTACTGACA | |
| | GCACTACAGGGCGCCTATGCATGTTAGTAGACCATGA | |
| | ATACAAGTACCCATATGTGTTAGGGCAAGGTCAAGA | |
| | TACTTTAGCCCCAGAACTTCCTATTTGGGTCTACTTTC | |
| | CCCCTCAATATGCTTACTTAACAGTAGGAGATGTTAA | |
| | CACACAAGGAATTTCTGGAGACAGCAAAAAATTAGC | |
| | AAGTGAAGAATCAGCATTTTATGTTTTGGAACACAGT | |
| | TCTTTTCAGCTTTTAGGTACAGGAGGTACAGCAACTA | |
| | TGTCTTATAAGTTTCCTCCAGTGCCCCCAGAAAATTT | |
| | AGAGGGCTGCAGTCAACACTTTTATGAGATGTACAAT | |
| | CCCTTATACGGATCCCGCTTAGGGGTTCCTGACACAT | |
| | TAGGAGGTGACCCAAAATTTAGATCTTTAACACATGA | |
| | AGACCATGCAATTCAGCCCCAAAACTTCATGCCAGG | |
| | GCCACTAGTAAACTCAGTGTCTACAAAGGAGGGAGA | |
| | CAGCTCTAATACTGGAGCTGGGAAAGCCTTAACAGG | |
| | CCTTAGCACAGGTACCTCTCAAAACACTAGAATATCC | |
| | TTACGCCCGGGGCCAGTGTCTCAGCCGTACCACCACT | |
| | GGGACACAGATAAATATGTCACAGGAATAAATGCTA | |
| | TTTCTCATGGTCAGACCACTTATGGTAACGCTGAAGA | |
| | CAAAGAGTATCAGCAAGGAGTGGGTAGATTTCCAAA | |
| | TGAAAAAGAACAGCTAAAACAGTTACAGGGTTTAAA | |
| | CATGCACACCTACTTTCCCAATAAAGGAACCCAGCAA | |
| | TATACAGATCAAATTGAGCGCCCCCTAATGGTGGGTT | |
| | CTGTATGGAACAGAAGAGCCCTTCACTATGAAAGCC | |
| | AGCTGTGGAGTAAAATTCCAAATTTAGATGACAGTTT | |
| | TAAAACTCAGTTTGCAGCCTTAGGAGGATGGGGTTTG | |
| | CATCAGCCACCTCCTCAAATATTTTTAAAAATATTAC | |
| | CACAAAGTGGGCCAATTGGAGGTATTAAATCAATGG | |
| | GAATTACTACCTTAGTTCAGTATGCCGTGGGAATTAT | |
| | GACAGTAACCATGACATTTAAATTGGGGCCCCGTAA | |
| | AGCTACGGGACGGTGGAATCCTCAACCTGGAGTATA | |
| | TCCCCCGCACGCAGCAGGTCATTTACCATATGTACTA | |
| | TATGACCCTACAGCTACAGATGCAAAACAACACCAC | |
| | AGACATGGATATGAAAAGCCTGAAGAATTGTGGACA | |
| | GCCAAAAGCCGTGTGCACCCATTGTAACTCGAGGCAT | |
| | GCGGTACCAAGCTTGTCGAGAAGTACTAGAGGATCA | |
| | TAATCAGCCATACCACATTTGTAGAGGTTTTACTTGC | |
| | TTTAAAAAACCTCCCACACCTCCCCCTGAACCTGAAA | |
| | CATAAAATGAATGCAATTGTTGTTGTTAACTTGTTTA | |
| | TTGCAGCTTATAATGGTTACAAATAAAGCAATAGCAT | |
| | CACAAATTTCACAAATAAAGCATTTTTTTCACTGCAT | |
| | TCTAGTTGTGGTTTGTCCAAACTCATCAATGTATCTTA | |
| | TCATGTCTGGATC | |
| Exemplary B19 | GATCCAGACATGATAAGATACATTGATGAGTTTGGAC | SEQ ID |
| Construct 4 | AAACCACAACTAGAATGCAGTGAAAAAAATGCTTTA | NO: 32 |
| comprising an | TTTGTGAAATTTGTGATGCTATTGCTTTATTTGTAACC | |
| erythroparvovirus | ATTATAAGCTGCAATAAACAAGTTAACAACAACAAT | |
| B19 VP1 capsid | TGCATTCATTTTATGTTTCAGGTTCAGGGGGAGGTGT | |
| nucleotide | GGGAGGTTTTTTAAAGCAAGTAAAACCTCTACAAATG | |
| sequence | TGGTATGGCTGATTATGATCCTCTAGTACTTCTCGAC | |
| | AAGCTTGGTACCGCATGCCTCGAGTTACAATGGGTGC | |
| | ACACGGCTTTTGGCTGTCCACAATTCTTCAGGCTTTTC | |
| | ATATCCATGTCTGTGGTGTTGTTTTGCATCTGTAGCTG | |
| | TAGGGTCATATAGTACATATGGTAAATGACCTGCTGC | |
| | GTGCGGGGGATATACTCCAGGTTGAGGATTCCACCGT | |
| | CCCGTAGCTTTACGGGGCCCCAATTTAAATGTCATGG | |
| | TTACTGTCATAATTCCCACGGCATACTGAACTAAGGT | |
| | AGTAATTCCCATTGATTTAATACCTCCAATTGGCCCA | |
| | CTTTGTGGTAATATTTTTAAAAATATTTGAGGAGGTG | |
| | GCTGATGCAAACCCCATCCTCCTAAGGCTGCAAACTG | |
| | AGTTTTAAAACTGTCATCTAAATTTGGAATTTTACTCC | |
| | ACAGCTGGCTTTCATAGTGAAGGGCTCTTCTGTTCCA | |
| | TACAGAACCCACCATTAGGGGGCGCTCAATTTGATCT | |
| | GTATATTGCTGGGTTCCTTTATTGGGAAAGTAGGTGT | |
| | GCATGTTTAAACCCTGTAACTGTTTTAGCTGTTCTTTT | |
| | TCATTTGGAAATCTACCCACTCCTTGCTGATACTCTTT | |
| | GTCTTCAGCGTTACCATAAGTGGTCTGACCATGAGAA | |
| | ATAGCATTTATTCCTGTGACATATTTATCTGTGTCCCA | |
| | GTGGTGGTACGGCTGAGACACTGGCCCCGGGCGTAA | |
| | GGATATTCTAGTGTTTTGAGAGGTACCTGTGCTAAGG | |
| | CCTGTTAAGGCTTTCCCAGCTCCAGTATTAGAGCTGT | |
| | CTCCCTCCTTTGTAGACACTGAGTTTACTAGTGGCCCT | |
| | GGCATGAAGTTTTGGGGCTGAATTGCATGGTCTTCAT | |
| | GTGTTAAAGATCTAAATTTTGGGTCACCTCCTAATGT | |
| | GTCAGGAACCCCTAAGCGGGATCCGTATAAGGGATT | |
| | GTACATCTCATAAAAGTGTTGACTGCAGCCCTCTAAA | |
| | TTTTCTGGGGGCACTGGAGGAAACTTATAAGACATAG | |
| | TTGCTGTACCTCCTGTACCTAAAAGCTGAAAAGAACT | |
| | GTGTTCCAAAACATAAAATGCTGATTCTTCACTTGCT | |
| | AATTTTTTGCTGTCTCCAGAAATTCCTTGTGTGTTAAC | |
| | ATCTCCTACTGTTAAGTAAGCATATTGAGGGGGAAAG | |
| | TAGACCCAAATAGGAAGTTCTGGGGCTAAAGTATCTT | |
| | GACCTTGCCCTAACACATATGGGTACTTGTATTCATG | |
| | GTCTACTAACATGCATAGGCGCCCTGTAGTGCTGTCA | |
| | GTAACCTGCACCCCCCCTCCAGTTTTGTCTGTAACAT | |
| | CCTTAACAGCAATTTCTGATATGGTTACAGTTAAAGC | |
| | ATCAGGAGCTATACTTCCATAATTTTCAATTAAGTGC | |
| | TGAAACTCTAAAGGTGAAAAAAATAAGTTTAAAGCA | |
| | TTAAAATCTAAATATCTCCATGGGGTTGAGTATCCCA | |
| | TTATGGGACTAATGGTGCAAACCTTTGCCTCCTTTCC | |
| | ACTGGCATTGTGGCAGCTACTTGCTGCGGGAGAAAA | |
| | CACCTTATAATGGTGCTCTGGGTCATATGGAATTAAA | |
| | AACTGCCTGGAAAATGTACAAGTCACAGAGTTGGCA | |
| | CTAAAAGTGGCCCCCTCACTCCACATGCTTTTGACAG | |
| | GATTACTGCCCCCCCCTCCTGCACCAGTGCTGGCTTC | |
| | TGCAGAATTAACTGAAGTCATGCTTGGGTATTTTTCT | |
| | GAGGCGTTGTAAGCGGGAACTTCCGGCAAACTTCCTT | |
| | GAAAATGGGCCACAGGGGCAGCTGCACCTTTTAAAG | |
| | TAAAGTAGTCTTTTACTACTTGTGCTTGAAACCCAGT | |
| | TTCGTTTTTTATATTTTTTAAAAGCTCTTCGTCTGCTA | |
| | CAGTCCAATGAGTATATGGATTTATTCCGAGCTTAGC | |
| | GAGTTGGCTATACCTAAAGTCGTGAATCCTTGCAGCA | |
| | CTGTCAACAGCACTTTGCGGGGGCCCAGCTTGTAGCT | |
| | CGTTGCCAGGCCCAACGTAGTTAGTACCGGGTAGTTG | |
| | TACGCTAACTTGCCCAGGCTTGTGTAAGTCTTCACTA | |
| | GATAATACTGCGTCTTCTCCTCTAGGTTCTGCGTGACT | |
| | GCTACTGGATGATAAGGCGTGGGGGTGGTCAGATAA | |
| | CTGTCCGTGACTTTGAAAATGATGACTATATAAGTCT | |
| | GGAGAATTTTTAAGGTTATTTTTAATGCGAGCAACTA | |
| | AGTCAAACAGAGAGGATGGGTTTTCTAGGGGATTAT | |
| | CTAAAGAAATATTATAATGATCTTTTAATATTTGAAT | |
| | AAGCTCTAAGTCTGTTCCAGTAACCTTTTCGTAAAAT | |
| | TCCACAAATTGCTGATACACAGCTTTAGCAAATTCGT | |
| | CGTCACTTTCCCACCACTTGCCACTTTCTTTACTCGTA | |
| | TTTATAGGTTTTTTTATTACAAAACTGTTACGAAAAC | |
| | AGTAAAATACTTATTTATTTGCGAGATGGTTATCATT | |
| | TTAATTATCTCCATGATCTATTAATATTCCGGAGTATA | |
| | CGTATACTCCGGAATATTAATAGGCGAAACACGCAC | |
| | GGCGCGCGCACGCAGCTTAGCACAAACGCGTCGTTG | |
| | CACGCGCCCACCGCTAACCGCAGGCCAATCGGTCGG | |
| | CCGGCCTCATATCCGCTCACCAGCCGCGTCCTATCGG | |
| | GCGCGGCTTCCGCGCCCATTTTGAATAAATAAACGAT | |
| | AACGCCGTTGGTGGCGTGAGGCATGTAAAAGGTTAC | |
| | ATCATTATCTTGTTCGCCATCCGGTTGGTATAAATAG | |
| | ACGTTCATGTTGGTTTTTGTTTCAGTTGCAAGTTGGCT | |
| | GCGGCGCGCGCAGCACCTTTGCATTCCGGATTATTCA | |
| | TACCGTCCCACCATCGGGCGCGGATCTGCCGCCCTGG | |
| | CGGGGTTTTACGAGATTGTGATTAAGGTCCCCAGCGA | |
| | CCTTGACGAGCATCTGCCCGGCATTTCTGACAGCTTT | |
| | GTGAACTGGGTGGCCGAGAAGGAGTGGGAGTTGCCG | |
| | CCAGATTCTGACTTGGATCTGAATCTGATTGAGCAGG | |
| | CACCCCTGACCGTGGCCGAGAAGCTGCAGCGCGACT | |
| | TTCTGACGGAGTGGCGCCGTGTGAGTAAGGCCCCGG | |
| | AGGCCCTTTTCTTTGTGCAATTTGAGAAGGGAGAGAG | |
| | CTACTTCCACTTACACGTGCTCGTGGAAACCACCGGG | |
| | GTGAAATCCTTAGTTTTGGGACGTTTCCTGAGTCAGA | |
| | TTCGCGAAAAACTGATTCAGAGAATTTACCGCGGGAT | |
| | CGAGCCGACTTTGCCAAACTGGTTCGCGGTCACAAAG | |
| | ACCAGAAACGGCGCCGGAGGCGGGAACAAGGTGGTG | |
| | GACGAGTGCTACATCCCCAATTACTTGCTCCCCAAAA | |
| | CCCAGCCTGAACTCCAGTGGGCGTGGACTAATTTAGA | |
| | ACAGTATTTAAGCGCCTGTTTGAATCTCACGGAGCGT | |
| | AAACGGTTGGTGGCGCAGCATCTGACGCACGTGTCG | |
| | CAGACGCAGGAGCAGAACAAAGAGAATCAGAATCCC | |
| | AATTCTGACGCGCCGGTGATCAGATCAAAAACTTCAG | |
| | CCAGGTACATGGAGCTGGTCGGGTGGCTCGTGGACA | |
| | AGGGGATTACCTCGGAGAAGCAGTGGATCCAGGAGG | |
| | ACCAGGCCTCATACATCTCCTTCAATGCGGCCTCCAA | |
| | CTCGCGGTCCCAAATCAAGGCTGCCTTGGACAATGCG | |
| | GGAAAGATTATGAGCCTGACTAAAACCGCCCCCGAC | |
| | TACCTGGTGGGCCAGCAGCCCGTGGAGGACATTTCCA | |
| | GCAATCGGATTTATAAAATTTTGGAACTAAACGGGTA | |
| | CGATCCCCAATATGCGGCTTCCGTCTTTCTGGGATGG | |
| | GCCACGAAAAAGTTCGGCAAGAGGAACACCATCTGG | |
| | CTGTTTGGGCCTGCAACTACCGGGAAGACCAACATCG | |
| | CGGAGGCCATAGCCCACACTGTGCCCTTCTACGGGTG | |
| | CGTAAACTGGACCAATGAGAACTTTCCCTTCAACGAC | |
| | TGTGTCGACAAGATGGTGATCTGGTGGGAGGAGGGG | |
| | AAGATGACCGCCAAGGTCGTGGAGTCGGCCAAAGCC | |
| | ATTCTCGGAGGAAGCAAGGTGCGCGTGGACCAGAAA | |
| | TGCAAGTCCTCGGCCCAGATAGACCCGACTCCCGTGA | |
| | TCGTCACCTCCAACACCAACATGTGCGCCGTGATTGA | |
| | CGGGAACTCAACGACCTTCGAACACCAGCAGCCGTT | |
| | GCAAGACCGGATGTTCAAATTTGAACTCACCCGCCGT | |
| | CTGGATCATGACTTTGGGAAGGTCACCAAGCAGGAA | |
| | GTCAAAGACTTTTTCCGGTGGGCAAAGGATCACGTGG | |
| | TTGAGGTGGAGCATGAATTCTACGTCAAAAAGGGTG | |
| | GAGCCAAGAAAAGACCCGCCCCCAGTGACGCAGATA | |
| | TAAGTGAGCCCAAACGGGTGCGCGAGTCAGTTGCGC | |
| | AGCCATCGACGTCAGACGCGGAAGCTTCGATCAACT | |
| | ACGCAGACAGGTACCAAAACAAATGTTCTCGTCACG | |
| | TGGGCATGAATCTGATGCTGTTTCCCTGCAGACAATG | |
| | CGAGAGAATGAATCAGAATTCAAATATCTGCTTCACT | |
| | CACGGACAGAAAGACTGTTTAGAGTGCTTTCCCGTGT | |
| | CAGAATCTCAACCCGTTTCTGTCGTCAAAAAGGCGTA | |
| | TCAGAAACTGTGCTACATTCATCATATCATGGGAAAG | |
| | GTGCCAGACGCTTGCACTGCCTGCGATCTGGTCAATG | |
| | TGGATTTGGATGACTGCATCTTTGAACAATAAGAGCT | |
| | CACTAGTCGCGGCCGCTTTCGAATCTAGAGCCTGCAG | |
| | TCTCGACAAGCTTGTCGAGAAGTACTAGAGGATCATA | |
| | ATCAGCCATACCACATTTGTAGAGGTTTTACTTGCTTT | |
| | AAAAAACCTCCCACACCTCCCCCTGAACCTGAAACAT | |
| | AAAATGAATGCAATTGTTGTTGTTAACTTGTTTATTG | |
| | CAGCTTATAATGGTTACAAATAAAGCAATAGCATCAC | |
| | AAATTTCACAAATAAAGCATTTTTTTCACTGCATTCT | |
| | AGTTGTGGTTTGTCCAAACTCATCAATGTATCTTATC | |
| | ATGTCTGGATC | |
| Exemplary B19 | GACATTGATTATTGACTAGTTATTAATAGTAATCAAT | SEQ ID |
| Construct 5 | TACGGGGTCATTAGTTCATAGCCCATATATGGAGTTC | NO: 33 |
| comprising a | CGCGTTACATAACTTACGGTAAATGGCCCGCCTGGCT | |
| codon-optimized | GACCGCCCAACGACCCCCGCCCATTGACGTCAATAAT | |
| erythroparvovirus | GACGTATGTTCCCATAGTAACGCCAATAGGGACTTTC | |
| B19 VP1 capsid | CATTGACGTCAATGGGTGGACTATTTACGGTAAACTG | |
| nucleotide | CCCACTTGGCAGTACATCAAGTGTATCATATGCCAAG | |
| sequence | TACGCCCCCTATTGACGTCAATGACGGTAAATGGCCC | |
| | GCCTGGCATTATGCCCAGTACATGACCTTATGGGACT | |
| | TTCCTACTTGGCAGTACATCTACGTATTAGTCATCGCT | |
| | ATTACCATGGTGATGCGGTTTTGGCAGTACATCAATG | |
| | GGCGTGGATAGCGGTTTGACTCACGGGGATTTCCAAG | |
| | TCTCCACCCCATTGACGTCAATGGGAGTTTGTTTTGG | |
| | CACCAAAATCAACGGGACTTTCCAAAATGTCGTAAC | |
| | AACTCCGCCCCATTGACGCAAATGGGCGGTAGGCGT | |
| | GTACGGTGGGAGGTCTATATAAGCAGAGCTCTCTGGC | |
| | TAACTAGAGAACCCACTGCTTACTGGCTTATCGAAAT | |
| | TAATACGACTCACTATAGGGAGACCCAAGCTTGGTAC | |
| | CGGACTCTAGAGGATCCGGTACTCGAGGAACTGAAA | |
| | AACCAGAAAGTTAACTGGTAAGTTTAGTCTTTTTGTC | |
| | TTTTATTTCAGGTCCCGGATCCGGTGGTGGTGCAAAT | |
| | CAAAGAACTGCTCCTCAGTGGATGTTGCCTTTACTTC | |
| | TAGGCCTGTACGGAAGTGTTACTTCTGCTCTAAAAGC | |
| | TGCGGAATTGTACCCGCGGAAGCTTCCTAGGCCGCCA | |
| | CCATGAGCAAGGAGAGCGGCAAGTGGTGGGAGAGCG | |
| | ACGACGAGTTCGCCAAGGCCGTGTACCAGCAGTTCGT | |
| | GGAGTTCTACGAGAAGGTGACCGGCACCGACCTGGA | |
| | GCTGATCCAGATCCTGAAGGACCACTACAACATCAG | |
| | CCTGGACAACCCCCTGGAGAACCCCAGCAGCCTGTTC | |
| | GACCTGGTGGCCCGCATCAAGAACAACCTGAAGAAC | |
| | AGCCCCGACCTGTACAGCCACCACTTCCAGAGCCACG | |
| | GCCAGCTGAGCGACCACCCCCACGCCCTGAGCAGCA | |
| | GCAGCAGCCACGCCGAGCCCCGCGGCGAGGACGCCG | |
| | TGCTGAGCAGCGAGGACCTGCACAAGCCCGGCCAGG | |
| | TGAGCGTGCAGCTGCCCGGCACCAACTACGTGGGCC | |
| | CCGGCAACGAGCTGCAGGCCGGCCCCCCCCAGAGCG | |
| | CCGTGGACAGCGCCGCCCGCATCCACGACTTCCGCTA | |
| | CAGCCAGCTGGCCAAGCTGGGCATCAACCCCTACAC | |
| | CCACTGGACCGTGGCCGACGAGGAGCTGCTGAAGAA | |
| | CATCAAGAACGAGACCGGCTTCCAGGCCCAGGTGGT | |
| | GAAGGACTACTTCACCCTGAAGGGCGCCGCCGCCCC | |
| | CGTGGCCCACTTCCAGGGCAGCCTGCCCGAGGTGCCC | |
| | GCCTACAACGCCAGCGAGAAGTACCCCAGCATGACC | |
| | AGCGTGAACAGCGCCGAGGCCAGCACCGGCGCCGGC | |
| | GGCGGCGGCAGCAACCCCGTGAAGAGCATGTGGAGC | |
| | GAGGGCGCCACCTTCAGCGCCAACAGCGTGACCTGC | |
| | ACCTTCAGCCGCCAGTTCCTGATCCCCTACGACCCCG | |
| | AGCACCACTACAAGGTGTTCAGCCCCGCCGCCAGCA | |
| | GCTGCCACAACGCCAGCGGCAAGGAGGCCAAGGTGT | |
| | GCACCATCAGCCCCATCATGGGCTACAGCACCCCCTG | |
| | GCGCTACCTGGACTTCAACGCCCTGAACCTGTTCTTC | |
| | AGCCCCCTGGAGTTCCAGCACCTGATCGAGAACTACG | |
| | GCAGCATCGCCCCCGACGCCCTGACCGTGACCATCAG | |
| | CGAGATCGCCGTGAAGGACGTGACCGACAAGACCGG | |
| | CGGCGGCGTGCAGGTGACCGACAGCACCACCGGCCG | |
| | CCTGTGCATGCTGGTGGACCACGAGTACAAGTACCCC | |
| | TACGTGCTGGGCCAGGGCCAGGACACCCTGGCCCCC | |
| | GAGCTGCCCATCTGGGTGTACTTCCCCCCCCAGTACG | |
| | CCTACCTGACCGTGGGCGACGTGAACACCCAGGGCA | |
| | TCAGCGGCGACAGCAAGAAGCTGGCCAGCGAGGAGA | |
| | GCGCCTTCTACGTGCTGGAGCACAGCAGCTTCCAGCT | |
| | GCTGGGCACCGGCGGCACCGCCACCATGAGCTACAA | |
| | GTTCCCCCCCGTGCCCCCCGAGAACCTGGAGGGCTGC | |
| | AGCCAGCACTTCTACGAGATGTACAACCCCCTGTACG | |
| | GCAGCCGCCTGGGCGTGCCCGACACCCTGGGCGGCG | |
| | ACCCCAAGTTCCGCAGCCTGACCCACGAGGACCACG | |
| | CCATCCAGCCCCAGAACTTCATGCCCGGCCCCCTGGT | |
| | GAACAGCGTGAGCACCAAGGAGGGCGACAGCAGCA | |
| | ACACCGGCGCCGGCAAGGCCCTGACCGGCCTGAGCA | |
| | CCGGCACCAGCCAGAACACCCGCATCAGCCTGCGCC | |
| | CCGGCCCCGTGAGCCAGCCCTACCACCACTGGGACA | |
| | CCGACAAGTACGTGACCGGCATCAACGCCATCAGCC | |
| | ACGGCCAGACCACCTACGGCAACGCCGAGGACAAGG | |
| | AGTACCAGCAGGGCGTGGGCCGCTTCCCCAACGAGA | |
| | AGGAGCAGCTGAAGCAGCTGCAGGGCCTGAACATGC | |
| | ACACCTACTTCCCCAACAAGGGCACCCAGCAGTACA | |
| | CCGACCAGATCGAGCGCCCCCTGATGGTGGGCAGCG | |
| | TGTGGAACCGCCGCGCCCTGCACTACGAGAGCCAGC | |
| | TGTGGAGCAAGATCCCCAACCTGGACGACAGCTTCA | |
| | AGACCCAGTTCGCCGCCCTGGGCGGCTGGGGCCTGC | |
| | ACCAGCCCCCCCCCCAGATCTTCCTGAAGATCCTGCC | |
| | CCAGAGCGGCCCCATCGGCGGCATCAAGAGCATGGG | |
| | CATCACCACCCTGGTGCAGTACGCCGTGGGCATCATG | |
| | ACCGTGACCATGACCTTCAAGCTGGGCCCCCGCAAG | |
| | GCCACCGGCCGCTGGAACCCCCAGCCCGGCGTGTAC | |
| | CCCCCCCACGCCGCCGGCCACCTGCCCTACGTGCTGT | |
| | ACGACCCCACCGCCACCGACGCCAAGCAGCACCACC | |
| | GCCACGGCTACGAGAAGCCCGAGGAGCTGTGGACCG | |
| | CCAAGAGCCGCGTGCACCCCCTGTAATAACTCGAGC | |
| | ATGCATCTAGAGGTACATCTAGATAGAGCTCGCTGAT | |
| | CAGCCTCGACTGTGCCTTCTAGTTGCCAGCCATCTGT | |
| | TGTTTGCCCCTCCCCCGTGCCTTCCTTGACCCTGGAAG | |
| | GTGCCACTCCCACTGTCCTTTCCTAATAAAATGAGGA | |
| | AATTGCATCGCATTGTCTGAGTAGGTGTCATTCTATT | |
| | CTGGGGGGTGGGGTGGGGCAGGACAGCAAGGGGGA | |
| | GGATTGGGAAGACAATAGCAGGCATGCTGGGGA | |
| Exemplary B19 | GACATTGATTATTGACTAGTTATTAATAGTAATCAAT | SEQ ID |
| Construct 6 | TACGGGGTCATTAGTTCATAGCCCATATATGGAGTTC | NO: 34 |
| comprising a | CGCGTTACATAACTTACGGTAAATGGCCCGCCTGGCT | |
| codon-optimized | GACCGCCCAACGACCCCCGCCCATTGACGTCAATAAT | |
| erythroparvovirus | GACGTATGTTCCCATAGTAACGCCAATAGGGACTTTC | |
| B19 VP2 capsid | CATTGACGTCAATGGGTGGACTATTTACGGTAAACTG | |
| nucleotide | CCCACTTGGCAGTACATCAAGTGTATCATATGCCAAG | |
| sequence | TACGCCCCCTATTGACGTCAATGACGGTAAATGGCCC | |
| | GCCTGGCATTATGCCCAGTACATGACCTTATGGGACT | |
| | TTCCTACTTGGCAGTACATCTACGTATTAGTCATCGCT | |
| | ATTACCATGGTGATGCGGTTTTGGCAGTACATCAATG | |
| | GGCGTGGATAGCGGTTTGACTCACGGGGATTTCCAAG | |
| | TCTCCACCCCATTGACGTCAATGGGAGTTTGTTTTGG | |
| | CACCAAAATCAACGGGACTTTCCAAAATGTCGTAAC | |
| | AACTCCGCCCCATTGACGCAAATGGGCGGTAGGCGT | |
| | GTACGGTGGGAGGTCTATATAAGCAGAGCTCTCTGGC | |
| | TAACTAGAGAACCCACTGCTTACTGGCTTATCGAAAT | |
| | TAATACGACTCACTATAGGGAGACCCAAGCTTGGTAC | |
| | CGGACTCTAGAGGATCCGGTACTCGAGGAACTGAAA | |
| | AACCAGAAAGTTAACTGGTAAGTTTAGTCTTTTTGTC | |
| | TTTTATTTCAGGTCCCGGATCCGGTGGTGGTGCAAAT | |
| | CAAAGAACTGCTCCTCAGTGGATGTTGCCTTTACTTC | |
| | TAGGCCTGTACGGAAGTGTTACTTCTGCTCTAAAAGC | |
| | TGCGGAATTGTACCCGCGGTTGAGGAACCTGTTAAGA | |
| | TGACCAGCGTGAACAGCGCCGAGGCCAGCACCGGCG | |
| | CCGGCGGCGGCGGCAGCAACCCCGTGAAGAGCATGT | |
| | GGAGCGAGGGCGCCACCTTCAGCGCCAACAGCGTGA | |
| | CCTGCACCTTCAGCCGCCAGTTCCTGATCCCCTACGA | |
| | CCCCGAGCACCACTACAAGGTGTTCAGCCCCGCCGCC | |
| | AGCAGCTGCCACAACGCCAGCGGCAAGGAGGCCAAG | |
| | GTGTGCACCATCAGCCCCATCATGGGCTACAGCACCC | |
| | CCTGGCGCTACCTGGACTTCAACGCCCTGAACCTGTT | |
| | CTTCAGCCCCCTGGAGTTCCAGCACCTGATCGAGAAC | |
| | TACGGCAGCATCGCCCCCGACGCCCTGACCGTGACCA | |
| | TCAGCGAGATCGCCGTGAAGGACGTGACCGACAAGA | |
| | CCGGCGGCGGCGTGCAGGTGACCGACAGCACCACCG | |
| | GCCGCCTGTGCATGCTGGTGGACCACGAGTACAAGT | |
| | ACCCCTACGTGCTGGGCCAGGGCCAGGACACCCTGG | |
| | CCCCCGAGCTGCCCATCTGGGTGTACTTCCCCCCCCA | |
| | GTACGCCTACCTGACCGTGGGCGACGTGAACACCCA | |
| | GGGCATCAGCGGCGACAGCAAGAAGCTGGCCAGCGA | |
| | GGAGAGCGCCTTCTACGTGCTGGAGCACAGCAGCTTC | |
| | CAGCTGCTGGGCACCGGCGGCACCGCCACCATGAGC | |
| | TACAAGTTCCCCCCCGTGCCCCCCGAGAACCTGGAGG | |
| | GCTGCAGCCAGCACTTCTACGAGATGTACAACCCCCT | |
| | GTACGGCAGCCGCCTGGGCGTGCCCGACACCCTGGG | |
| | CGGCGACCCCAAGTTCCGCAGCCTGACCCACGAGGA | |
| | CCACGCCATCCAGCCCCAGAACTTCATGCCCGGCCCC | |
| | CTGGTGAACAGCGTGAGCACCAAGGAGGGCGACAGC | |
| | AGCAACACCGGCGCCGGCAAGGCCCTGACCGGCCTG | |
| | AGCACCGGCACCAGCCAGAACACCCGCATCAGCCTG | |
| | CGCCCCGGCCCCGTGAGCCAGCCCTACCACCACTGGG | |
| | ACACCGACAAGTACGTGACCGGCATCAACGCCATCA | |
| | GCCACGGCCAGACCACCTACGGCAACGCCGAGGACA | |
| | AGGAGTACCAGCAGGGCGTGGGCCGCTTCCCCAACG | |
| | AGAAGGAGCAGCTGAAGCAGCTGCAGGGCCTGAACA | |
| | TGCACACCTACTTCCCCAACAAGGGCACCCAGCAGTA | |
| | CACCGACCAGATCGAGCGCCCCCTGATGGTGGGCAG | |
| | CGTGTGGAACCGCCGCGCCCTGCACTACGAGAGCCA | |
| | GCTGTGGAGCAAGATCCCCAACCTGGACGACAGCTT | |
| | CAAGACCCAGTTCGCCGCCCTGGGCGGCTGGGGCCT | |
| | GCACCAGCCCCCCCCCCAGATCTTCCTGAAGATCCTG | |
| | CCCCAGAGCGGCCCCATCGGCGGCATCAAGAGCATG | |
| | GGCATCACCACCCTGGTGCAGTACGCCGTGGGCATCA | |
| | TGACCGTGACCATGACCTTCAAGCTGGGCCCCCGCAA | |
| | GGCCACCGGCCGCTGGAACCCCCAGCCCGGCGTGTA | |
| | CCCCCCCCACGCCGCCGGCCACCTGCCCTACGTGCTG | |
| | TACGACCCCACCGCCACCGACGCCAAGCAGCACCAC | |
| | CGCCACGGCTACGAGAAGCCCGAGGAGCTGTGGACC | |
| | GCCAAGAGCCGCGTGCACCCCCTGTAATAACTCGAG | |
| | CATGCATCTAGAGATCTAGATAGAGCTCGCTGATCAG | |
| | CCTCGACTGTGCCTTCTAGTTGCCAGCCATCTGTTGTT | |
| | TGCCCCTCCCCCGTGCCTTCCTTGACCCTGGAAGGTG | |
| | CCACTCCCACTGTCCTTTCCTAATAAAATGAGGAAAT | |
| | TGCATCGCATTGTCTGAGTAGGTGTCATTCTATTCTG | |
| | GGGGGTGGGGTGGGGCAGGACAGCAAGGGGGAGGA | |
| | TTGGGAAGACAATAGCAGGCATGCTGGGGA | |
| Exemplary AAV2 | ATCATGGAGATAATTAAAATGATAACCATCTCGCAA | SEQ ID |
| Construct 1 | ATAAATAAGTATTTTACTGTTTTCGTAACAGTTTTGTA | NO: 35 |
| comprising an | ATAAAAAAACCTATAAATATTCCGGATTATTCATACC | |
| AAV2 VP1 | GTCCCACCATCGGGCGCGGATCTCCTGTTAAGACGGC | |
| capsid nucleotide | TGCCGACGGTTATCTACCCGATTGGCTCGAGGACACT | |
| sequence | CTCTCTGAAGGAATAAGACAGTGGTGGAAGCTCAAA | |
| | CCTGGCCCACCACCACCAAAGCCCGCAGAGCGGCAT | |
| | AAGGACGACAGCAGGGGTCTTGTGCTTCCTGGGTAC | |
| | AAGTACCTCGGACCCTTCAACGGACTCGACAAGGGA | |
| | GAGCCGGTCAACGAGGCAGACGCCGCGGCCCTCGAG | |
| | CACGACAAAGCCTACGACCGGCAGCTCGACAGCGGA | |
| | GACAACCCGTACCTCAAGTACAACCACGCCGACGCG | |
| | GAGTTTCAGGAGCGCCTTAAAGAAGATACGTCTTTTG | |
| | GGGGCAACCTCGGACGAGCAGTCTTCCAGGCGAAAA | |
| | AGAGGGTTCTTGAACCTCTGGGCCTGGTTGAGGAACC | |
| | TGTTAAGACGGCTCCGGGAAAAAAGAGGCCGGTAGA | |
| | GCACTCTCCTGTGGAGCCAGACTCCTCCTCGGGAACC | |
| | GGAAAGGCGGGCCAGCAGCCTGCAAGAAAAAGATTG | |
| | AATTTTGGTCAGACTGGAGACGCAGACTCAGTACCTG | |
| | ACCCCCAGCCTCTCGGACAGCCACCAGCAGCCCCCTC | |
| | TGGTCTGGGAACTAATACGATGGCTACAGGCAGTGG | |
| | CGCACCAATGGCAGACAATAACGAGGGCGCCGACGG | |
| | AGTGGGTAATTCCTCGGGAAATTGGCATTGCGATTCC | |
| | ACATGGATGGGCGACAGAGTCATCACCACCAGCACC | |
| | CGAACCTGGGCCCTGCCCACCTACAACAACCACCTCT | |
| | ACAAACAAATTTCCAGCCAATCAGGAGCCTCGAACG | |
| | ACAATCACTACTTTGGCTACAGCACCCCTTGGGGGTA | |
| | TTTTGACTTCAACAGATTCCACTGCCACTTTTCACCAC | |
| | GTGACTGGCAAAGACTCATCAACAACAACTGGGGAT | |
| | TCCGACCCAAGAGACTCAACTTCAAGCTCTTTAACAT | |
| | TCAAGTCAAAGAGGTCACGCAGAATGACGGTACGAC | |
| | GACGATTGCCAATAACCTTACCAGCACGGTTCAGGTG | |
| | TTTACTGACTCGGAGTACCAGCTCCCGTACGTCCTCG | |
| | GCTCGGCGCATCAAGGATGCCTCCCGCCGTTCCCAGC | |
| | AGACGTCTTCATGGTGCCACAGTATGGATACCTCACC | |
| | CTGAACAACGGGAGTCAGGCAGTAGGACGCTCTTCA | |
| | TTTTACTGCCTGGAGTACTTTCCTTCTCAGATGCTGCG | |
| | TACCGGAAACAACTTTACCTTCAGCTACACTTTTGAG | |
| | GACGTTCCTTTCCACAGCAGCTACGCTCACAGCCAGA | |
| | GTCTGGACCGTCTCATGAATCCTCTCATCGACCAGTA | |
| | CCTGTATTACTTGAGCAGAACAAACACTCCAAGTGGA | |
| | ACCACCACGCAGTCAAGGCTTCAGTTTTCTCAGGCCG | |
| | GAGCGAGTGACATTCGGGACCAGTCTAGGAACTGGC | |
| | TTCCTGGACCCTGTTACCGCCAGCAGCGAGTATCAAA | |
| | GACATCTGCGGATAACAACAACAGTGAATACTCGTG | |
| | GACTGGAGCTACCAAGTACCACCTCAATGGCAGAGA | |
| | CTCTCTGGTGAATCCGGGCCCGGCCATGGCAAGCCAC | |
| | AAGGACGATGAAGAAAAGTTTTTTCCTCAGAGCGGG | |
| | GTTCTCATCTTTGGGAAGCAAGGCTCAGAGAAAACA | |
| | AATGTGGACATTGAAAAGGTCATGATTACAGACGAA | |
| | GAGGAAATCAGGACAACCAATCCCGTGGCTACGGAG | |
| | CAGTATGGTTCTGTATCTACCAACCTCCAGAGAGGCA | |
| | ACAGACAAGCAGCTACCGCAGATGTCAACACACAAG | |
| | GCGTTCTTCCAGGCATGGTCTGGCAGGACAGAGATGT | |
| | GTACCTTCAGGGGCCCATCTGGGCAAAGATTCCACAC | |
| | ACGGACGGACATTTTCACCCCTCTCCCCTCATGGGTG | |
| | GATTCGGACTTAAACACCCTCCTCCACAGATTCTCAT | |
| | CAAGAACACCCCGGTACCTGCGAATCCTTCGACCACC | |
| | TTCAGTGCGGCAAAGTTTGCTTCCTTCATCACACAGT | |
| | ACTCCACGGGACAGGTCAGCGTGGAGATCGAGTGGG | |
| | AGCTGCAGAAGGAAAACAGCAAACGCTGGAATCCCG | |
| | AAATTCAGTACACTTCCAACTACAACAAGTCTGTTAA | |
| | TGTGGACTTTACTGTGGACACTAATGGCGTGTATTCA | |
| | GAGCCTCGCCCCATTGGCACCAGATACCTGACTCGTA | |
| | ATCTGTAACTCGAGGCATGCGGTACCAAGCTTGTCGA | |
| | GAAGTACTAGAGGATCATAATCAGCCATACCACATTT | |
| | GTAGAGGTTTTACTTGCTTTAAAAAACCTCCCACACC | |
| | TCCCCCTGAACCTGAAACATAAAATGAATGCAATTGT | |
| | TGTTGTTAACTTGTTTATTGCAGCTTATAATGGTTACA | |
| | AATAAAGCAATAGCATCACAAATTTCACAAATAAAG | |
| | CATTTTTTTCACTGCATTCTAGTTGTGGTTTGTCCAAA | |
| | CTCATCAATGTATCTTATCATGTCTGGATC | |

In some embodiments, the host cell is derived from a species of lepidoptera, e.g., Spodoptera frugiperda, Spodoptera littoralis, Spodoptera exigua, or Trichoplusia ni. In some embodiments, the host cell is an insect cell. In some embodiments, the host cell is Sf9. In some embodiments, the at least one vector is a baculoviral vector, a viral vector, or a plasmid. In some embodiments, the at least one vector is a baculoviral vector. In some embodiments, subclones of lepidopteran cell lines that demonstrate enhanced vector yield on a per cell or per volume basis are used. In some embodiments, modified lepidopteran cell lines with an integrated copy of a nonstructural protein (e.g., NS, NS1, and/or NS2), Rep, VP, and/or vector genome, singly or in combinations, are used. A host cell line, in some embodiments, is “cured” of endogenous or contaminating or adventitious insect viruses such as the Spodoptera rhabdovirus.

In some embodiments, a VP1 capsid protein comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NO: 9. In some embodiments, a VP2 capsid protein comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NO: 11. In some embodiments, a capsid protein comprises a structural protein VP1 capsid protein, VP2 capsid protein, or combination thereof. VP2 may be present in excess of VP1.

In some embodiments, a VP1 capsid protein is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 6, SEQ ID NO: 7, or SEQ ID NO: 8. In some embodiments, a VP1 capsid protein is encoded by a nucleic acid that is codon-optimized for expression. In some embodiments, the VP2 is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 10. In some embodiments, the VP2 is encoded by a nucleic acid that is codon-optimized for expression.

In some embodiments, an ITR comprises (a) a dependoparvovirus ITR (b) an AAV ITR, optionally an AAV2 ITR, and/or (c) a human erythroparvovirus B19 ITR. In some embodiments, an ITR comprises (a) a dependoparvovirus ITR (b) an AAV ITR, optionally an AAV2 ITR, and/or (c) an erythroparvovirus ITR. In certain embodiments, an ITR is a terminal palindrome with Rep binding elements and trs that is structurally similar to a wild-type ITR (e.g., for B19). An ITR, in some embodiments, is from AAV1, 2, 3, etc. In certain embodiments, an ITR has the AAV2 RBE and trs. In some embodiments, an ITR is a chimera of different AAVs. In some embodiments, an ITR and Rep protein are from AAV5. In some embodiments, an ITR is synthetic and is comprised of RBE motifs and trs GGTTGG, AGTTGG, AGTTGA, RRTTRR. The typical T-shaped structure of the terminal palindrome consisting of the B/B′ and C/C′ stems may also be synthetically modified with substitutions and insertions that maintain the overall secondary structure based on folding prediction (available at URI, (http) of unafold.rna.albany.edu/?q=mfold/DNA-Folding-Form). The stability of an ITR secondary structure is designated by the Gibbs free energy, delta G, with lower values, i.e., more negative, indicating greater stability. The full-length, 145 nt ITR has a computed ΔG=−69.91 kcal/mol. The B and C stems: GCCCGGGCAAAGCCCGGGCGTCGGGCGACCTTTGGTCGCCCG have ΔG=−22.44 kcal/mol. Substitutions and insertions that result in a structure with ΔG=−15 kcal/mol to −30 kcal/mol are functionally equivalent and not distinct from the wild-type dependoparvovirus ITRs.

In some embodiments, the at least one expression control sequence for expression in a host cell (e.g., an insect cell, e.g., a mammalian cell) comprises: (a) a promoter, and/or (b) a Kozak-like expression control sequence. In some embodiments, the promoter comprises: (a) an immediate early promoter of an animal DNA virus, (b) an immediate early promoter of an insect virus, or (c) a host cell promoter. In some embodiments, the animal DNA virus is cytomegalovirus (CMV), erythroparvovirus, or AAV. In some embodiments, the animal DNA virus is erythroparvovirus B19. In some embodiments, the insect virus is a lepidopteran virus or a baculovirus, optionally wherein the baculovirus is Autographa californica multicapsid nucleopolyhedrovirus (AcMNPV). In some embodiments, the promoter is a polyhedrin (polh) or immediately early 1 gene (IE-1) promoter. In some embodiments, the nucleotide sequence comprising at least one replication protein of an AAV (e.g., AAV2) comprises a nucleotide sequence encoding Rep52 and/or Rep78.

In some embodiments, provided herein are host cells comprising at least one vector, comprising: (i) a nucleotide sequence comprising at least one ITR nucleotide sequence, (ii) a nucleotide sequence comprising at least one gene encoding erythroparvovirus B19 VP1 capsid protein and/or VP2 capsid protein operably linked to at least one expression control sequence for expression in a host cell (e.g., an insect cell, e.g., a mammalian cell), and (iii) a nucleotide sequence comprising (A) at least one replication protein of erythroparvovirus B19 operably linked to at least one expression control sequence for expression in a host cell, (B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or (C) a combination of (A) and (B). In some embodiments, the vector is a host cell-compatible vector that comprises a promoter that facilitates the expression of a nucleic acid in host cells. In some embodiments, at least one of (i), (ii), (iii) (A), (iii) (B), and (iii) (C) is stably integrated in the host cell genome.

In some embodiments, host cells comprise the at least one gene encoding at least one erythroparvovirus capsid protein, wherein the erythroparvovirus is selected from primate erythroparvovirus 4 (pig-tailed macaque parvovirus), primate erythroparvovirus 3 (rhesus macaque parvovirus), primate erythroparvovirus 2 (simian parvovirus), rodent erythroparvovirus 1, ungulate erythroparvovirus 1, or a genotypic variant thereof.

In some embodiments, host cells comprise the at least one gene encoding a capsid protein(s) of erythroparvovirus B19 or a genotypic variant thereof.

In some embodiments, the at least one replication protein is a nonstructural protein (e.g., NS, NS1, and/or NS2) of an erythroparvovirus or a genotypic variant thereof. In some embodiments, the at least one replication protein is a nonstructural protein (e.g., NS, NS1, and/or NS2) of a human erythroparvovirus B19 or a genotypic variant thereof. In some embodiments, a host cell is derived from a species of lepidoptera, e.g., Spodoptera frugiperda, Spodoptera littoralis, Spodoptera exigua, or Trichoplusia ni. In some embodiments, a host cell is Sf9. In some embodiments, the at least one vector is a baculoviral vector, a viral vector, or a plasmid. In some embodiments, the at least one vector is a baculoviral vector.

In some embodiments, a VP1 capsid protein comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NOs: 9, 14, 17, 19, 21, or 25. In some embodiments, the VP2 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the SEQ ID NOs: 11, 15, or 22. In some embodiments, the capsid protein comprises structural proteins VP1 and VP2. VP2 may be present in excess of VP1.

In some embodiments, a VP1 capsid protein is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 6, SEQ ID NO: 7, or SEQ ID NO: 8. In some embodiments, a VP1 capsid protein is encoded by a nucleic acid that is codon-optimized for expression. In some embodiments, the VP2 is encoded by a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO: 10. In some embodiments, the VP2 is encoded by a nucleic acid that is codon-optimized for expression.

In some embodiments, the ITR comprises (a) an AAV ITR, optionally an AAV2 ITR, and/or (b) an erythroparvovirus ITR. In some embodiments, the ITR comprises (a) an AAV ITR, optionally an AAV2 ITR, and/or (b) a human erythroparvovirus B19 ITR. In some embodiments, the at least one expression control sequence for expression in a host cell (e.g., an insect cell, e.g., a mammalian cell) comprises: (a) a promoter, and/or (b) a Kozak-like expression control sequence. In some embodiments, the promoter comprises: (a) an immediate early promoter of an animal DNA virus, (b) an immediate early promoter of an insect virus, or (c) a host cell promoter. In some embodiments, the animal DNA virus is cytomegalovirus (CMV), erythroparvovirus, or AAV. In some embodiments, the animal DNA virus is erythroparvovirus B19. In some embodiments, an insect virus is a lepidopteran virus or a baculovirus, optionally wherein the baculovirus is Autographa californica multicapsid nucleopolyhedrovirus (AcMNPV). In some embodiments, a promoter is a polyhedrin (polh) or immediately early 1 gene (IE-1) promoter. In some embodiments, a nucleotide sequence comprising at least one replication protein of an AAV (e.g., AAV2) comprises a nucleotide sequence encoding Rep52 and/or Rep78.

While less efficient than the methods described herein, a recombinant virion may also be produced using a mammalian cell, e.g., Grieger et al (2016) Mol Ther 24:287-297).

### EXAMPLES

### Example 1: Construction of Recombinant Virions Containing Erythroparvovirus Capsid Proteins

The present Example describes construction of recombinant virions comprising erythroparovirus capsid proteins. In some embodiments, erythroparovirus capsid proteins are human erythroparovirus B19 capsid proteins. In some embodiments, erythroparovirus capsid proteins are erythroparovirus capsid proteins described herein.

### A Nucleic Acid for a Recombinant Virion

A vector genome design consists of inverted terminal repeats (ITRs), e.g., the ITR conformers of the AAV terminal palindrome and an expression or transcription cassette. Generic expression cassettes comprise of regulatory elements, typically characterized as enhancer and promoter elements. A region transcribed by an RNA polymerase complex comprises of cis acting regulatory elements e.g., TATA-box, and 5′ untranslated exonic sequences, intronic sequences, translated exonic sequences, 3′ untranslated region, poly-adenylation signal sequence. Post-transcriptional elements include a Kozak motif for translational initiation and the woodchuck hepatitis virus post-transcriptional regulatory element. A specific vector is chemically synthesized using a commercial service provider and ligated into a plasmid for propagation in Escherichia coli. A plasmid minimally contains multiple cloning sites, at least one antibiotic resistance gene, a plasmid origin of replication, and sequences to facilitate recombination into a baculovirus genome. Two commonly used approaches are: 1. A bacterial system in which the E. coli harbors a baculovirus genome (bacmid) that uses transposase mediated recombination to transfer the plasmid genes into the bacmid. E. coli with the recombinant bacmid is detectable by growth on agar plates prepared with selective media. The “positive” colonies are expanded in suspension culture medium and the bacmid harvested after about 3 days post-inoculation. Sf9 cells are then transfected with the bacmid which in the permissive host cell, produce infectious, recombinant baculovirus particles. b2. Alternatively, the vector DNA is inserted into a shuttle plasmid that has several hundred basepairs of baculovirus DNA flanking the insert. Co-transfection of Sf9 cells with the shuttle plasmid and linearized baculovirus subgenomic DNA restores the deleted baculovirus elements producing infectious, recombinant baculovirus. The ≤6 kb vector DNA resides in the baculovirus genome (ca. 135 kb) and is propagated as baculovirus unless the Sf9 cell expresses the parvovirus non-structural or Rep proteins. The Rep protein then acts on the ITR allowing resolution of the vector and baculovirus genomes where the vector genome then replicates autonomously of the baculovirus genome (FIG. 1B).

### Nucleic Acid Composed of DNA

DNA can be either single-stranded or self-complimentary (i.e., intramolecular duplex). As illustrated in FIG. 1B, Rep-mediated replication of the vector DNA proceeds through several intermediates. These replicative intermediates are processed into single-stranded virion genomes, however, the fecundity of products may overwhelm processing into single-stranded virion genomes. In this case, the replicative intermediate consisting of an intramolecular duplex molecule, represented as the RFm (FIG. 1B), is packaged into the parvovirus capsid. Packaging of the self-complementary vector genomes occurs despite the presence of functional ITRs.

DNA can have a Rep protein-dependent origin of replication (ori). The ori can consist of Rep binding elements (RBEs), and within a terminal palindrome. The terminal palindrome, referred to as the inverted terminal repeats (ITRs), can consist of an overall palindromic sequence with two internal palindromes. The ITR can have cis-acting motifs required for replication and encapsidation in capsids.

RBE represents Rep binding elements canonical GCTC; RBE′ represents non-canonical RBE, unpaired TTT at the tip of the ITR cross-arm; and trs represents terminal resolution site 5′AGTTGG, GGTTGG, etc. The catalytic tyrosine of Rep (Y156) cleaves the trs and forms a covalent link with the scissile, 5′thymidine. Mutation of the trs leads to inefficient or loss of cleavage resulting in self-complimentary DNA. Alternatively, self-complementary virion genomes result from encapsidation of the incomplete processing of the RFm.

### DNA Replication of an Erythroparvovirus Recombinant Virion

Replication utilizing AAV ITR-Parvovirus DNA replication is referred to as “rolling hairpin” replication. As single-stranded virion DNA, the ITRs form an energetically stable, T-shaped structure (FIG. 1A) that serves as a primer for DNA extension by the host-cell DNA polymerase complex (FIG. 1B). DNA synthesis is leading strand, processive process resulting in a duplex intermediate where the complementary strands are covalently linked through the ITR (FIG. 1B). The p5 Rep protein binds are structurally related to rolling-circle replication (RCR) proteins, bind to the ITR forming a multi-subunit complex. The helicase activity of the Rep proteins unwinds the ITR creating a single-stranded bubble with the terminal resolution site (5′-GGT|TGA-3′). The phosphodiester bond between the thymidines is attacked by the hydroxyl group of the Rep protein catalytic tyrosine (AAV2=Y156) forming a tyrosine-thymidine diester with the 5′-thymidine. A cellular DNA polymerase complex extends the newly created 3-OH at the terminal resolution site restoring the terminal sequence to the template strand (FIG. 1B). Resolution of the nucleoprotein complex occurs through an unknown process.

Replication utilizing erythroparvovirus terminal repeats and non-structural (NS) protein(s)-Erythroparvovirus replication is similar to AAV DNA replication, although the terminal palindromes are unique and require a specific NS protein for processing the replication intermediates. A recombinant erythroparvovirus vector genome may consist of erythroparvovirus termini flanking the transgene cassette. NS1 dependent rolling-hairpin replication process is similar to AAV Rep-dependent replication and capsid contain single stranded genomes of either polarity.

### Encapsidation

Encapsidation or packaging of DNA into an icosahedral virus capsid is an active process requiring a source of energy to overcome the repulsive force created by back-pressure of compressing DNA into a confined volume. ATPase activities of the NS/Rep proteins translate the stored chemical energy of the trinucleotide by hydrolyzing the gamma phosphate. Backpressure generated determines the length of DNA that can be accommodated in the capsid, i.e., the motive force of the ATPase/helicase can “push” up to 12 pN, for example, which may be reached once 4,800 nucleotides are packaged. AAV p19 Rep proteins are monomeric, non-processive helicases that are necessary for efficient encapsidation. Although there are scant data that support physical interactions between Rep and capsid, the overcoming the backpressure requires that stable interactions form between the packaging helicase(s) and the capsid. The nature of these interactions are unknown and nuclear factors may stabilize or mediate the interactions between the non-structural proteins and capsids. The phylogenetically related erythroparvovirus and dependoparvovirus capsids are divergent at the sequence level, therefore, the interactions between NS proteins and capsids of heterologous genera may not result in efficient encapsidation.

To improve the packaging of genomes comprising AAV2 genes into erythroparvovirus capsids, cognate NS proteins are co-expressed with the AAV Rep proteins in a permissive cell: AAV Rep78 and Rep 52 are required for vector DNA replication and erythroparvovirus NS proteins are involved in packaging.

### Example 2: Producing Erythroparvovirus Recombinant Virions Using Insect Cells

The present Example describes construction of recombinant virions comprising erythroparovirus capsid proteins. In some embodiments, erythroparovirus capsid proteins are human erythroparovirus B19 capsid proteins. In some embodiments, erythroparovirus capsid proteins are erythroparovirus capsid proteins described herein.

Sf9 cells are grown in serum-free insect cell culture medium (HyClone SFX-Insect Cell Culture Medium) and transferred from an erlenmyer shake flask (Corning) to a Wave single-use bioreactor (GE Healthcare). Cells density and viability are determined daily using a Cellometer Autor 2000 (Nexelcom). Volume is adjusted to maintain a cell density of 2 to 5 million cells per ml. At the final volume (10 L) and density of 2.5 million cells per ml, the baculovirus infected insect cells (BIICs) are added (cryopreserved, 100× concentrated cell “plugs”) 1:10,000 (v:v). The highly diluted BIICs release Rep-VP-Bac, NS-Bac, and vg-Bac that are at very low multiplicity of infection (MOI) and virtually no cells are co-infected during the primary infection. However, subsequent infection cycles release large numbers of each of the requisite baculovirus achieving a very high MOI ensuring that each cell is infected with numerous virus particles. The cells are maintained in culture for four days or until viability drops to ≤30%.

### Example 3: Purification of Erythroparvovirus Recombinant Virions

The present Example describes construction of recombinant virions comprising erythroparovirus capsid proteins. In some embodiments, erythroparovirus capsid proteins are human erythroparovirus B19 capsid proteins. In some embodiments, erythroparovirus capsid proteins are erythroparovirus capsid proteins described herein.

Recombinant erythroparvovirus particles are partitioned in both the cellular and extracellular fractions. To recover the maximum number of particles, the entire biomass including cell culture medium is processed. To release the intracellular erythroparvovirus particles, Triton-X 100 (x %) is added to the bioreactor with continued agitation for 1 hr. The temperature is increased from 27° C. to 37° C. then Benzonase (EMD Merck) or Turbonuclease (Accelagen, Inc.) is added (2 u per ml) to the bioreactor with continued agitation. The biomass is clarified using a staged depth filter, then filter sterilized (0.2 μm) and collected in a sterile bioprocessing bag. Recombinant erythroparvovirus particles are recovered using sequential column chromatography using immune-affinity chromatography medium and Q-Sepharose anion exchange. Chromatograms displaying and recording UV absorption, pH, and conductivity are used to determine completion of the washing and elution steps. Relative efficiency of each step is determined by western blot analysis and quantitatively by ddPCR or qPCR analysis aliquots of the input material (“Load”), the flow-through, the wash, and the elution.

Immune-affinity chromatography uses a “nanobody,” the VhH region of a single-domain immunoglobulin produced in llamas and other camelid species. To produce the nanobody, an antibody provider immunizes llamas with erythroparvovirus virus-like particles, i.e., assembled capsids with no virion genome. Erythroparvovirus VLPs are prepared in Sf9 cells infected with the VP-Bac and purified using cesium chloride isopycnic gradients, followed by size exclusion chromatography (Superdex 200). Following a prime (1×)/boost (2×) immunization protocol the antibody service provider bleeds the llama and isolates peripheral blood mononuclear cells or mRNA extracted from nucleated blood cells. Reverse transcription using primers specific for the conserved VhH CDR flanking regions (FR1 and FR 4) produces cDNA that is cloned into plasmids used to generate the T7Select 10-3b phage display library (EMD-Millipore). Following several rounds of panning to enrich for phage that interact with erythroparvovirus capsids, phage clones are isolated from plaques. E. coli infected with the recombinant phage are mixed into agarose and applied as an overlay onto LB-agar plates. The E. coli grow to confluency establishing a “lawn” where lysed bacteria and appear as plaques on the plate. To identify phage that bind to erythroparvovirus, nitrocellulose filters placed on surface of the agar plates to transfer proteins from the plaques to the filter. The filters are incubated with erythroparvovirus capsids modified with a covalently linked horseradish peroxidase (HRP) (EZLink Plus Activated Peroxidase Kit, ThermoFisher) and washed with phosphate buffered saline. HRP activity can be detected with either a chromogenic (Novex HRP Chromogenic Substrate, ThermoFisher) or chemiluminescent substrate (Pierce ECL Western Blotting Substrate, ThermoFisher). The sequences of the cDNA in the phage are determined and ligated into a bacterial expression plasmid and expressed with a 6×His tag for purification. The chelating column-purified nanobody is covalently linked to chromatography medium, NHS-activated Sepharose 4 Fast Flow (GE Healthcare).

Erythroparvovirus particles are recovered from the clarified Sf9 cell lysate by binding, washing, and eluting from the nanobody-Sepharose column. The efficiency of binding is determined by western blotting the column load and flow through. The wash step is considered complete when the UV280 nm absorbance returns to baseline (i.e., pre-load) values. An acidic pH shift releases erythroparvovirus particles are eluted from the nanobody-Sepharose medium. The eluate is collected in 50 nM Tris-Cl, pH 7.2 to neutralize the elution medium.

The concentration of erythroparvovirus vector particles is determined using erythroparvovirus specific ELISA and qPCR which can be used to estimate the percentage of filled particles, i.e., vector genome-containing.

### Example 4: Purification of CD34+ Cells

CD34+ cells for use in the disclosed methods can be purified according to suitable methods, such as those described in the following articles: Hayakama et al., Busulfan produces efficient human cell engraftment in NOD/LtSz-scid IL2Ry null mice, Stem Cells 27 (1): 175-182 (2009); Ochi et al., Multicolor Staining of Globin Subtypes Reveals Impaired Globin Switching During Erythropoiesis in Human Pluripotent Stem Cells, Stem Cells Translational Medicine 3:792-800 (2014); and McIntosh et al., Nonirradiated NOD,B6.SCID I/2ry^{-/-} Kit^{W41/W41} (NBSGW) Mice Support Multilineage Engraftment of Human Hematopoietic Cells, Stem Cell Reports 4:171-180 (2015).

### Example 5: In Vitro or Ex Vivo Transduction of Erythroid Progenitor Cells Using Erythroparvovirus Recombinant Virions

The present Example describes in vitro or ex vivo transduction of erythroid progenitor cells using erythryoparvovirus recombinant virions. In some embodiments, erythryoparvovirus recombinant virions are erythryoparvovirus B19 recombinant virions. In some embodiments, erythryoparvovirus recombinant virions are other erythryoparvovirus recombinant virions described herein.

The capacity of erythroparvovirus is approximately 110% of the wild-type virion 5.6 kb genome, which is about 6,160 nt in length, of which, approximately 300 nt required for the ITRs, leaving 5,860 nt for “cargo.” This represents 1 kb greater capacity than conventional adeno-associated virus vectors.

A recombinant erythroparvovirus is used to transduce erythroid progenitor cells. The affinity of erythroparvovirus for an erythroid specific globoside, P-antigen, provides an improved method to deliver therapeutic transgenes to erythroid progenitor cells and that gene replacement may be accomplished by genomic editing. Transgene expression in genotypically corrected cells facilitates rescue of the phenotype of the differentiated cells and lead to clinical improvement.

Hemoglobinopathies caused by gain of function mutations are inherited as autosomal recessive traits. Heterozygous individuals tend to be either asymptomatic or mildly affected, whereas individuals with mutations in both alleles are severely affected. Thus, correcting or replacing a single allele is clinically beneficial.

Since both beta-thalassemia and sickle cell disease (SCD) are caused by different mutations in the genes that express hemoglobin beta (HbB), a gene replacement strategy benefits patients with either disease. There are clinical studies for SCD using lentivirus vector (LV) that deliver the HbB expression cassette. The b-globin open reading frame (ORF) is regulated by the globin allele locus control region (LCR) and b-globin promoter. In order to fit into the LV, the minimal LCR has been mapped to three DNAse hypersensitive sites (HS) that inhibit DNA methylation and the formation of heterochromatin. Randomly integrating LV may integrate into heterochromatin resulting in shut-off of b-globin expression in the erythrocyte progenitor cells (e.g., erythroblasts), and thus, no phenotypic correction.

The LCR elements, HS, maintain the open, euchromatin structure of LV DNA regardless of integration site. However, the minimized LCR, compared to the b-globin ORF (441 bp and 147 codons) is relatively large limiting the virus vector delivery options.

Inserting the HbB cassette into a genomic safe harbor (GSH) locus. In contrast to transposable elements which constitute approximately 45% of the mammalian genome, heritable integrated parvovirus genomes (or endogenous virus elements, EVEs) occur in very few loci across hundreds of species. The EVEs are genomic markers of sites that tolerate insertion of foreign DNA without affecting embryogenesis, development, maturation, etc. on the short time-line and evolution/speciation on a geologic time-line. Presumably due to the disruptive effects of foreign DNA insertion, there are very few EVE loci that have accumulated in many diverse species over 100 million years. Despite the many species among the highly diverse phylogenetic taxa that harbor EVEs, there appear to be a limited number of genomic loci affected facilitating an empirical analysis of EVEs as GSHs in model systems, e.g., mouse. The conservation of the EVE loci among mammalian species allows us to determine the homologous sites in the human and mouse genomes. However, it is likely that not all GSHs will support long-term, stable expression all tissue types. Using in silico analysis, including RNAseq and ATACseq databases, GSH loci can be mapped to subgenomic regions that are actively expressed in the target tissue. Thus, for beta-globinopathies, erythroblasts are particularly interesting.

Utilizing GSH loci that are actively chromatin regions actively expressed chromatin in erythroblasts, circumvents the necessity of using the LCR elements to ensure euchromatinization where the LV integrated.

The process of homology directed repair (HDR) with a targeting nuclease improves the efficiency and specificity of recombination. “Homology arms” flanking the therapeutic gene, directs the vector DNA to the targeted locus. Recombination either by cellular DNA repair pathway enzymes, or an artificial process, e.g., CRISPR/Cas9 nuclease, integrates the transgene into a GSH.

In addition to b-globin promoter, other promoters have been used for long-term, high-level expression in numerous cell types and also in transgenic mouse strains.

For example, hemoglobin is a heterotetramer composed of 2× HbA and 2× HbB chains. In the absence of HbB, the HbA chain self-associates and form cytotoxic aggregates. The alpha-hemoglobin stabilizing protein (AHSP) is co-expressed in pro-erythrocytes to prevent aggregation of α-globin subunits. The AHSP promoter is highly active in erythrocyte precursors and is well characterized.

As another example, the CAG promoter enhancer is a synthetic promoter engineered from the cytomegalovirus enhancer fused to the chicken beta-globin promoter and exon 1 and intron 1 and splice acceptor of exon 2.

As another example, the MND promoter is active hematopoietic cells

As another example, the Wiskott-Aldrich promoter is active in hematopoietic cells.

As another example, the PKLR promoter is active in hematopoietic cells

Peripheral blood stem cells (PBSCs) are isolated by leukopheresis.

Cryopreserved peripheral blood cells in Hemofreeze bags are recovered by rapid thawing in a 37° C. water bath. These thawed cells are suspended in 4% HSA at 4° C. and washed twice by centrifugation at 450 g for 5 min at 4° C. The platelets are removed twice by overlaying on 10% HSA and centrifugation at 450 g for 15 min at 4° C. The erythrocytes are removed by overlaying on Ficoll-Hypaque (FH; 1.077 g/cm³; Pharmacia Fine Chemicals, Piscataway, NJ, USA) and centrifugation at 400 g for 25 min at 4° C. The interface mononuclear cells (Pl-, FH cells) are collected, washed twice in washing solution and resuspended in 4% HSA at 4° C. (MN cells). A nylon-fiber syringe (NF-S) is used to remove adherent cells. Five grams of NF is packed into a 50 ml disposable syringe. The mono nuclear cells were transferred to an additional 50 ml syringe and gently infused into the NF-S, then were incubated at 4° C. for 5 min. The MN cells are then collected into a 50 ml syringe through a plunger of the NF-S, and the cells are pooled in 50 ml of a conical tube. These pooled cells are centrifuged at 400 g for 5 min at 4° C., and resuspended in 4% HSA at 4° C. (NF cells). The cell suspension is then immediately processed for CD34+ selection on the Isolex Magnetic Cell Separation System (Isolex 50; Baxter Healthcare, Immunotherapy Division, Newbury, UK) following the manufacturer's instructions. Briefly, cells are incubated with 9C5 murine immunoglobulin G1 (IgG1) anti-human CD34 antibody (10 m g/1×10⁸ NF cells) for 15 min at 4° C. with slow end-over-end rotation. After sensitization, the cells are washed with 4% HSA at 4° C. to remove any excess/unbound antibody. The Dynabeads (Oslo, Norway) are then added to the washed, sensitized cells at a final bead/cell ratio of 1:10. After mixing at 4° C. for 30 min, the cell-bound microspheres and free microspheres become attached to the wall via the magnet (Dynal MPC-1, Dynal, Fort Lee, NJ, USA) and any free cells that do not bind to the microspheres are removed. This washing procedure is repeated twice with 4% HSA at 4° C. The linkage between Dynabeads and CD34+ cells is cleaved by a PR34+ Stem Cell Releasing Agent for 30 min at 4° C. The free Dynabeads are removed from the CD34+ cells via the magnet. D-PBS containing 1% ACD-A and 1% HSA at 25° C. is used for collection of cells. The resulted cell product is controlled by Flow cytometry.

Isolated fresh or cryopreserved CD34+ cells are thawed and immediately transduced with erythroparvovirus vectors in serum free medium. Two hours post-transduction, cells are switched to the expansion medium (IMDM, FBS, SCF, IL3, Epo, Dexamethasone, β-estradiol, β-mercaptoethanol) and grown at 5×10⁵ cells/mL. At day 10, cells are switched to the erythroid differentiation medium (IMDM, BSA, Insulin, Transferrin, Epo). All transgene expressions are determined either by western blotting, fluorescence microscopy or by flow cytometry.

### Example 6: In Vivo Transduction of Erythroid Progenitor Cells Using Erythroparvovirus Recombinant Virions

The present Example describes in vivo transduction of erythroid progenitor cells using erythryoparvovirus recombinant virions. In some embodiments, erythryoparvovirus recombinant virions are erythryoparvovirus B19 recombinant virions. In some embodiments, erythryoparvovirus recombinant virions are other erythryoparvovirus recombinant virions described herein.

An erythroparvovirus recombinant virion described in Example 5 is prepared. A erythroparvovirus recombinant virion is administered to a human subject who is in need of the transgene. A subject is administered with a recombinant virion by intravenous infusion or by a localized injection (e.g., bone marrow).

### Example 7: Exemplary Nucleotide Sequences Encoding an Erythroparvovirus VP1 Capsid Protein Showed Improved Infection

The present Example confirms production of recombinant virions comprising an exemplary erythroparvovirus VP1 capsid protein and a heterologous nucleic acid encoding a green fluorescent protein (GFP) using methods described herein.

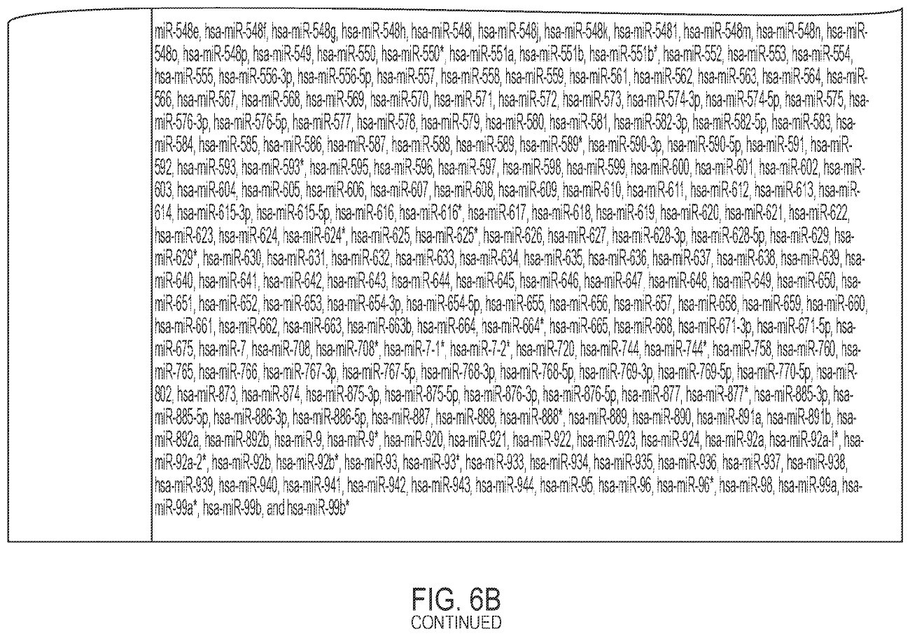

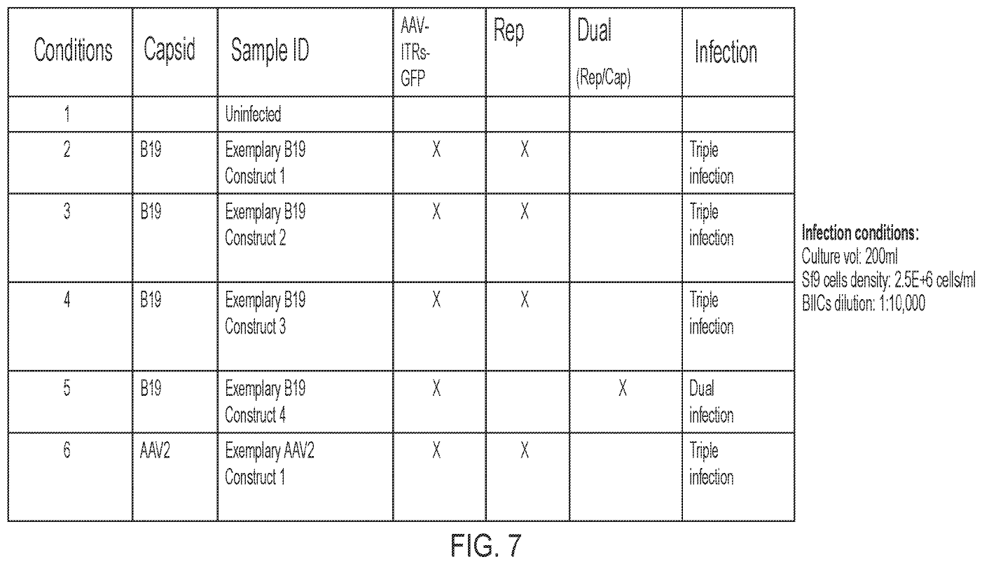

As shown in FIG. 7, in some embodiments, recombinant virion production methods include triple infection (e.g., AAV genome, cap, and rep). In some embodiments, recombinant virion production methods include double infection (e.g., AAV genome, rep/cap). In some embodiments, infection conditions comprise a culture volume of 200 ml, an Sf9 cells density of 2.5E+6 cells/ml, and a Baculovirus Infected Insect Cell (BIIC) dilution of 1:10,000.

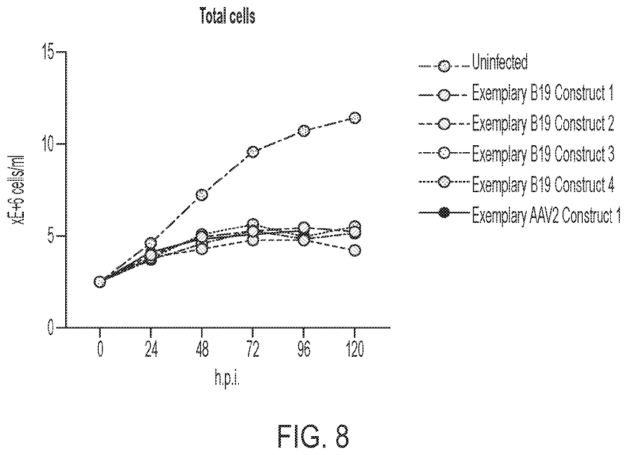

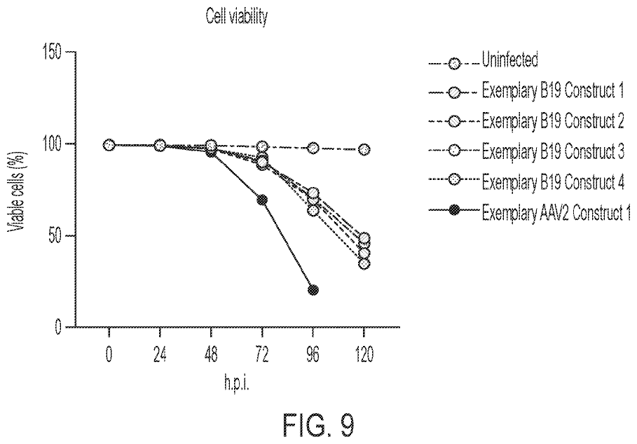

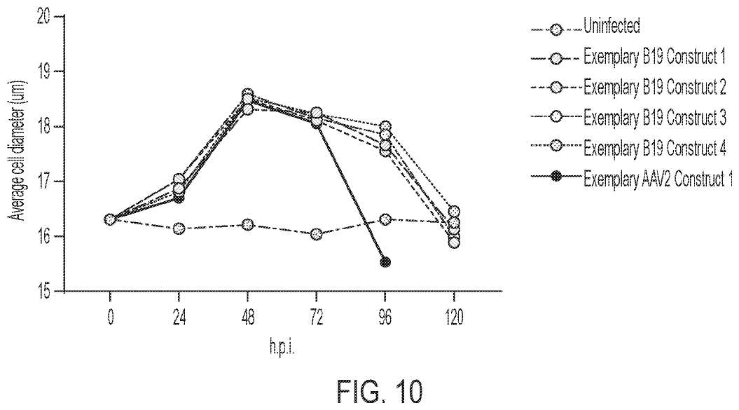

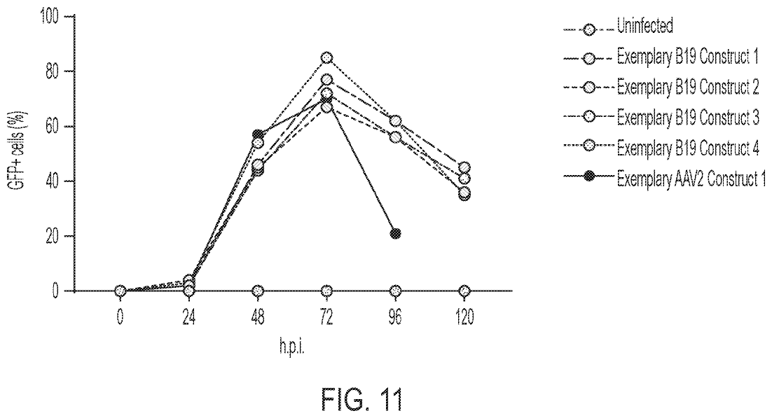

Infection kinetics of recombinant virions comprising exemplary erythroparvovirus B19 VP1 capsid proteins were evaluated in a BEV-Sf9 system and compared to formation of recombinant virions comprising an AAV2 VP1 capsid protein at 24, 48, 72, 96, and 120 hours post infection (hpi). Among other things, it is an insight of the present disclosure that baculovirus infection parameters show similar kinetics across different exemplary nucleotide sequences comprising at least one gene encoding an erythroparvovirus B19 VP1 capsid protein, as described herein. Recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) showed similar infection of total Sf9 cells over time relative to recombinant virions comprising an AAV2 VP1 capsid protein encoded by a nucleotide sequence according to SEQ ID NO: 35 (Exemplary AAV2 Construct 1), as shown in FIG. 8. Surprisingly, recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) showed improved cell viability in Sf9 cells, measured by percent viable cells, relative to recombinant virions comprising an AAV2 VP1 capsid protein encoded by a nucleotide sequence according to SEQ ID NO: 35 (Exemplary AAV2 Construct 1), as shown in FIG. 9. Moreover, recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) showed improved infection in Sf9 cells, as measured by average cell diameter, at 120 hours post infection (hpi) relative to recombinant virions comprising an AAV2 VP1 capsid protein encoded by a nucleotide sequence according to SEQ ID NO: 35 (Exemplary AAV2 Construct 1), as shown in FIG. 10. FIG. 11 shows that Sf9 cells comprising recombinant virions comprising an exemplary erythroparvovirus B19 VP1 capsid protein according to SEQ ID NOs: 29-32 (Exemplary B19 Construct 1, Exemplary B19 Construct 2, Exemplary B19 Construct 3, Exemplary B19 Construct 4, respectively) showed improved GFP expression relative to Sf9 cells comprising recombinant virions comprising an AAV2 VP1 capsid protein encoded by a nucleotide sequence according to SEQ ID NO: 35 (Exemplary AAV2 Construct 1). Without wishing to be bound to any theory, in some embodiments, it is believed recombinant virions comprising an AAV2 VP1 capsid protein show faster replication kinetics compared to recombinant virions comprising an exemplary erythroparvovirus B19 VP1 capsid protein.

It is an insight of the present Example that other cell types can be transduced using exemplary compositions, preparations, nucleotide sequences, recombinant virions, and population of cells comprising recombinant virions described herein.

Accordingly, the present Example confirms that recombinant virions comprising an exemplary erythroparvovirus VP1 capsid protein as described herein can be produced using methods as described by the present disclosure. Moreover, in some embodiments, the present Example confirms that an exemplary erythroparvovirus VP1 capsid protein described herein can exhibit improved cell viability, improved infection, and improved heterologous nucleic acid expression.

### Example 8: AAV2 Genome Rescue in Sf9 Cells Infected with Recombinant Virions Comprising an Erythroparvovirus VP1 Capsid Protein

Among other things, it is an insight of the present disclosure that amplification of AAV2 genomes is important for effective virion formation. The present Example confirms effective AAV2 genome rescue in cells comprising a recombinant virion comprising an exemplary erythroparvovirus VP1 capsid protein, an AAV replication (Rep) protein, and AAV ITRs via triple infection (e.g., AAV genome, capsid, rep) or double infection (e.g., AAV genome, rep/cap) according to methods described herein.

In some embodiments, Sf9 cells were co-infected with a baculovirus expression vector (BEV) comprising a nucleotide sequence encoding a functional AAV Rep protein, a BEV comprising a heterologous nucleic acid comprising AAV2 ITRs, and an exemplary nucleotide sequence encoding an exemplary erythroparvovirus B19 VP1 capsid protein according to SEQ ID NOs: 29-31 (Exemplary B19 Construct 1, Exemplary B19 Construct 2, Exemplary B19 Construct 3, respectively). In some embodiments, Sf9 cells were co-infected with a BEV comprising an exemplary dual nucleotide sequence encoding an exemplary erythroparvovirus B19 VP1 capsid protein and an AAV2 Rep protein according to SEQ ID NO: 32 (Exemplary B19 Construct 4), and a BEV comprising a heterologous nucleic acid comprising AAV2 ITRs.

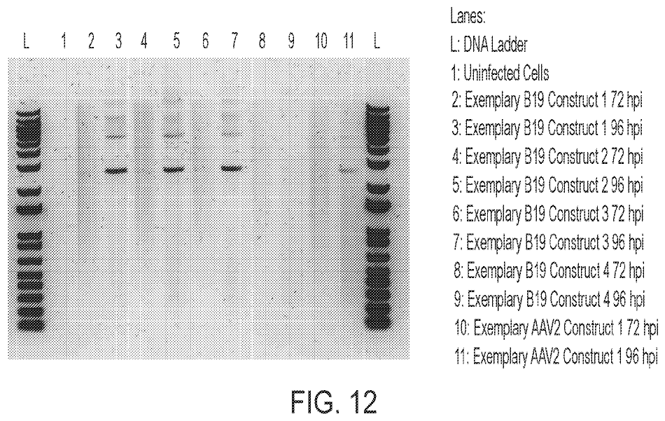

FIG. 12 shows rescue of an AAV2 genome in cells infected with recombinant virions comprising an exemplary erythroparvovirus B19 VP1 capsid protein encoded by a nucleotide sequence an according to SEQ ID NOs: 29-32 (Exemplary B19 Construct 1, Exemplary B19 Construct 2, Exemplary B19 Construct 3, Exemplary B19 Construct 4, respectively) and cells infected with recombinant virions comprising an AAV2 VP1 capsid protein encoded by a nucleotide sequence according to SEQ ID NO: 35 (Exemplary AAV2 Construct 1) via PCR analysis. Lower AAV2 genome rescue was observed in cells co-infected with an exemplary dual nucleotide sequence encoding an exemplary erythroparvovirus B19 VP1 capsid protein and AAV Rep protein according to SEQ ID NO: 32 (Exemplary B19 Construct 4), relative to cells infected with three BEVs, wherein one vector comprises a nucleotide sequence encoding an exemplary erythroparvovirus B19 VP1 capsid protein according to SEQ ID NOs: 29-31 (Exemplary B19 Construct 1, Exemplary B19 Construct 2, Exemplary B19 Construct 3, respectively). Control cells infected with recombinant virions comprising an AAV2 VP1 capsid protein encoded by a nucleotide sequence according to SEQ ID NO: 35 (Exemplary AAV2 Construct 1) showed genome rescue at 96 hpi.

It is an insight of the present Example that other cell types can be transduced using exemplary compositions, preparations, nucleotide sequences, recombinant virions, and population of cells comprising recombinant virions described herein.

Accordingly, the present Example confirms amplification of AAV genomes for effective recombinant virion formation as described herein.

### Example 9: Exemplary Nucleotide Sequences Encoding an Erythroparvovirus VP1 Capsid Protein Show High Virion Yields

The present Example confirms exemplary compositions, preparations, nucleotide sequences, recombinant virions, and population of cells comprising recombinant virions, and host cells for gene therapy and related methods as described herein show high recombinant virion yields.

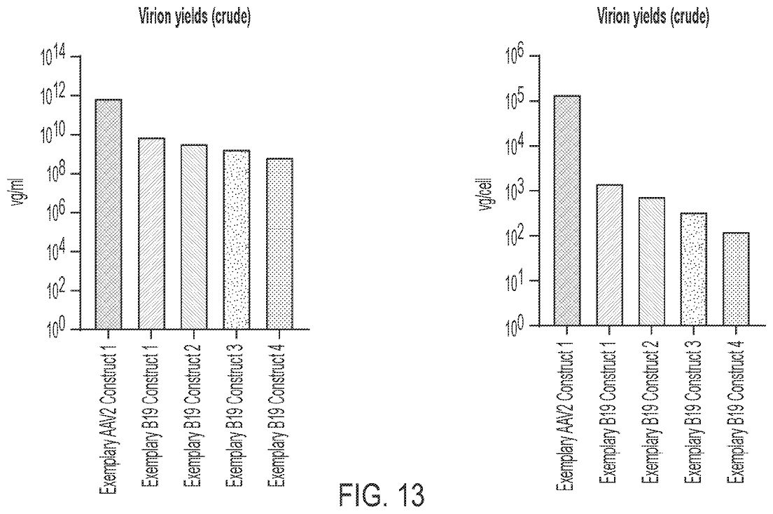

Formation of recombinant virions comprising an exemplary erythroparvovirus VP1 capsid protein was evaluated in a BEV-Sf9 system and compared to formation of recombinant virions comprising an AAV2 VP1 capsid protein, as shown in FIG. 13. Recombinant virion yields were measured for recombinant virions comprising an exemplary erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively) and for virions comprising an AAV2 VP1 capsid protein encoded by a nucleotide sequence according to SEQ ID NO.: 35 (Exemplary AAV2 Construct 1). Recombinant virions comprising an exemplary erythroparvovirus B19 VP1 capsid protein reached yields of ˜E+9 vg/ml or ˜E+3 vg/cell. Surprisingly, exemplary nucleotide sequences comprising at least one gene encoding an exemplary erythroparvovirus B19 VP1 capsid protein according to SEQ ID NOs: 29-32 (Exemplary B19 Construct 1, Exemplary B19 Construct 2, Exemplary B19 Construct 3, Exemplary B19 Construct 4, respectively) produced recombinant virion yields at similar level to an exemplary control nucleotide sequence encoding an AAV2 VP1 capsid protein according to SEQ ID NO: 35 (Exemplary AAV2 Construct 1).

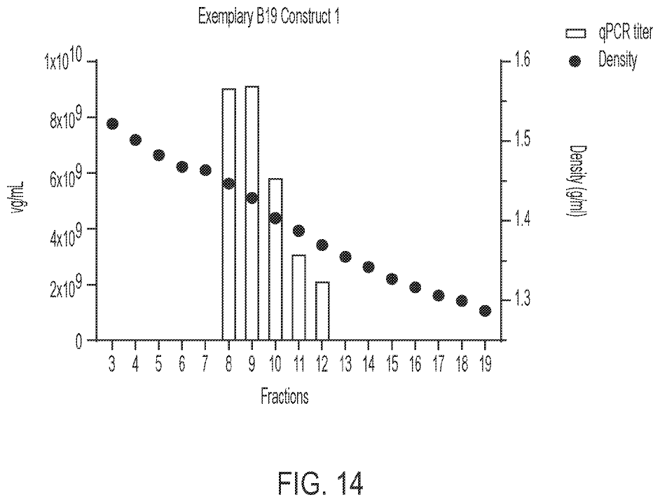

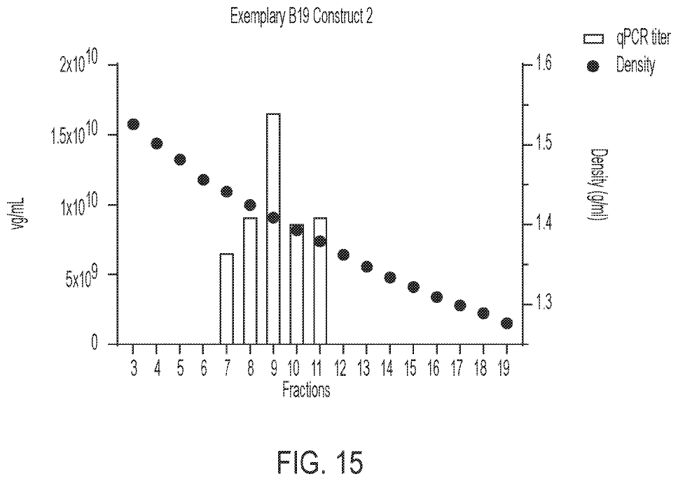

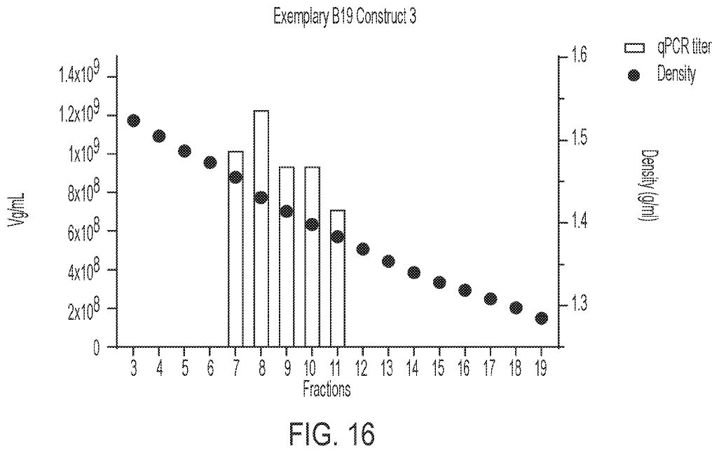

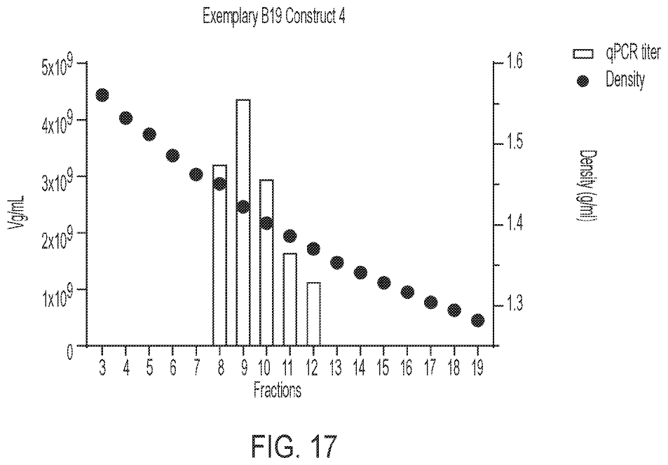

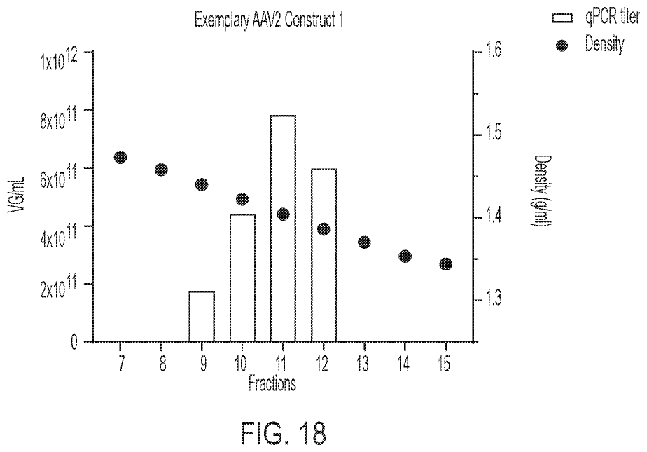

It is an insight of the present disclosure that wild-type full erythroparvovirus B19 virion buoyant density is 1.42-1.45 g/cm3 in CsCl. Recombinant virions comprising an exemplary erythroparvovirus B19 VP1 capsid protein were generated and tested in Sf9 cells according to standard protocols. Ultracentrifugation (UC) fractions with density values corresponding to full erythroparvovirus B19 recombinant virions were used to perform AAV genome quantification via qPCR. As shown in FIGS. 14-17, fractions comprising full recombinant virions were detected. qPCR data suggests the presence of full recombinant virions comprising an exemplary erythroparvovirus B19 VP1 capsid protein encoded by a nucleotide sequence according to SEQ ID NO: 29 (Exemplary B19 Construct 1) in fractions 8-9, as shown in FIG. 14. Full recombinant virions comprising an exemplary erythroparvovirus B19 VP1 capsid protein encoded by a VP1 capsid protein sequence according to SEQ ID NO: 30 (Exemplary B19 Construct 2) were detected in fraction 9, as shown in FIG. 15. Full recombinant virions comprising an exemplary erythroparvovirus B19 VP1 capsid protein encoded by a nucleotide sequence according to SEQ ID NO: 31 (Exemplary B19 Construct 3) were detected in fraction 8, as shown in FIG. 16. Full recombinant virions comprising an exemplary erythroparvovirus B19 VP1 capsid protein encoded by a nucleotide sequence according to SEQ ID NO: 32 (Exemplary B19 Construct 4) were detected in fraction 9, as shown in FIG. 17. Full recombinant virions comprising an AAV2 VP1 capsid protein encoded by a nucleotide sequence according to SEQ ID NO: 35 (Exemplary AAV2 Construct 1) were detected in fraction 11, as shown in FIG. 18.

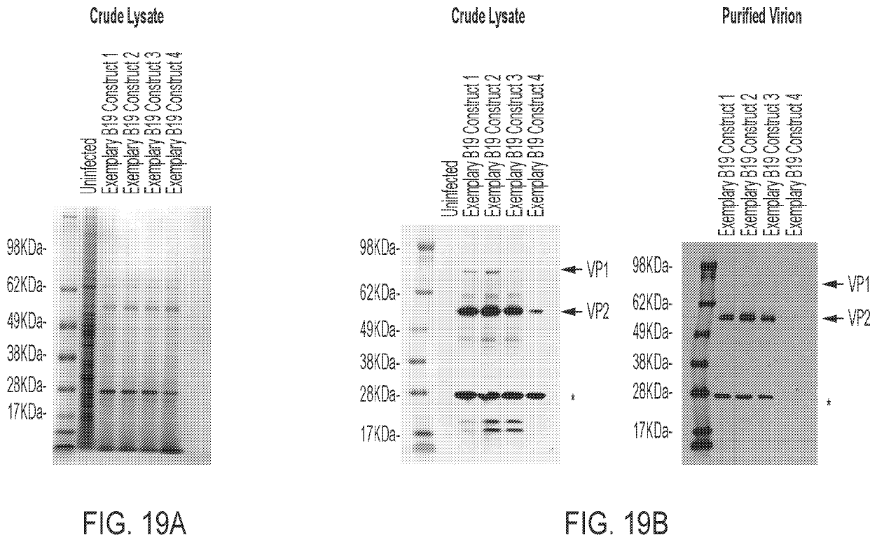

As shown in FIGS. 19A-19B, crude lysates and ultra-centrifuged (UC)-purified cell fractions were analyzed by western blot using an anti-VP2 capsid protein specific antibody. FIG. 19A shows the presence of erythroparvovirus B19 VP1 and VP2 capsid proteins in crude lysates of cells infected with recombinant virions comprising an erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4). FIG. 19B shows the presence of erythroparvovirus B19 VP1 and VP2 capsid proteins in crude lysates (left) and purified virions (right) from cells infected with recombinant virions comprising an erythroparvovirus B19 VP1 capsid protein encoded by a nucleotide sequence according to SEQ ID NOs: 29-32 (Exemplary B19 Construct 1, Exemplary B19 Construct 2, Exemplary B19 Construct 3, Exemplary B19 Construct 4, respectively). VP1 and VP2 capsid proteins were detected in crude lysates of cells infected with recombinant virions comprising an erythroparvovirus B19 VP1 capsid protein encoded by a nucleotide sequence according to SEQ ID NOs.: 29-31 (Exemplary B19 Construct 1, Exemplary B19 Construct 2, Exemplary B19 Construct 3, respectively). A faint VP2 capsid protein band was observed in crude lysates and UC-purified fractions of cells infected with recombinant virions comprising an erythroparvovirus B19 VP1 capsid protein encoded by a nucleotide sequence according to SEQ ID NO: 32 (Exemplary B19 Construct 4). Moreover, without wishing to be bound to any theory, an unspecific protein band (marked with *) observed in crude lysates and in UC-purified cell fractions, is believed to correspond to GFP protein (˜27 KDa) which has been observed to co-migrate with erythroparvovirus capsids.

It is an insight of the present Example that other cell types can be transduced using exemplary compositions, preparations, nucleotide sequences, recombinant virions, and population of cells comprising recombinant virions described herein.

Accordingly, the present Example confirms that exemplary nucleotide sequences comprising at least one gene encoding an exemplary erythroparvovirus VP1 capsid protein as described herein show high virion yields. The present Example also confirms that exemplary nucleotide sequences comprising at least one gene encoding an erythroparvovirus VP1 capsid protein as described herein produce full recombinant virions. Moreover, the present Example also confirms that recombinant virions described herein can deliver transgene(s) that are robustly expressed in cells (or populations of cells) as described herein.

### Example 10: Exemplary Nucleotide Sequences Encoding an Erythroparvovirus VP1 Capsid Protein Showed Transduction in Human Cells

The present Example confirms that exemplary compositions, preparations, nucleotide sequences, recombinant virions, and population of cells comprising recombinant virions, and host cells for gene therapy and related methods described herein showed transduction in human cells as described herein.

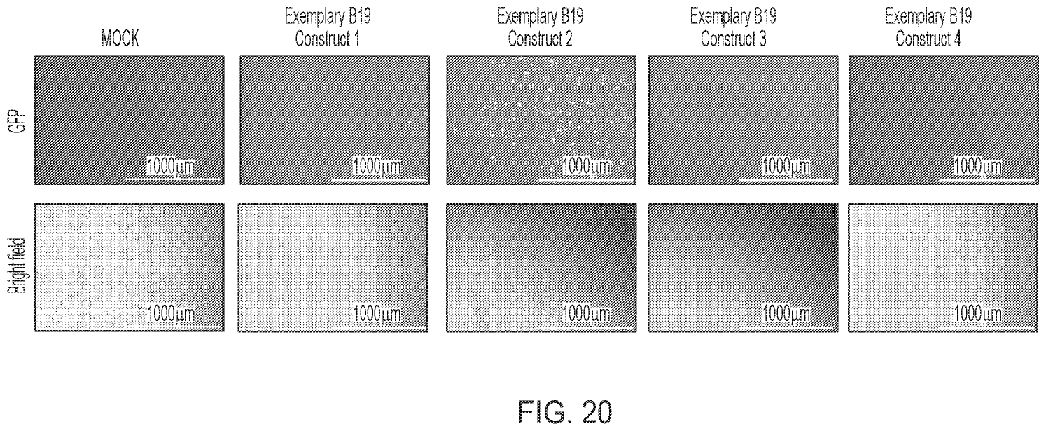

FIG. 20 shows fluorescence (top) and phase imaging (bottom) of transduction of recombinant virions comprising an exemplary erythroparvovirus B19 VP1 capsid protein encoded by an exemplary nucleotide sequence according to SEQ ID NOs: 29-32 (Exemplary B19 Construct 1, Exemplary B19 Construct 2, Exemplary B19 Construct 3, Exemplary B19 Construct 4, respectively) and a heterologous nucleic acid encoding GFP in K562 cells. qPCR signal in ultra-centrifuged (UC)-purified cell fractions of cells infected with recombinant virions comprising an erythroparvovirus B19 VP1 capsid protein encoded by a nucleotide sequence according to SEQ ID NO: 32 (Exemplary B19 Construct 4), as shown in FIG. 17, suggests packaging of an AAV2 genome, however, not at high enough level to drive detectable transduction in K562 cells, as shown in FIG. 20.

Cells infected with recombinant virions comprising an erythroparvovirus B19 VP1 capsid protein encoded by a nucleotide sequence according to SEQ ID NO: 30 (Exemplary B19 Construct 2) showed highest expression of VP1 capsid protein (FIG. 19B), which correlated with higher biological activity observed in K562 cells.

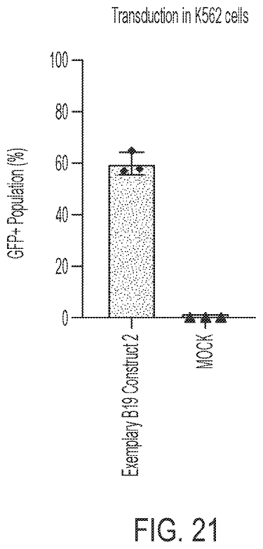

Recombinant virions comprising an erythroparvovirus B19 VP1 capsid protein encoded by a nucleotide sequence according to SEQ ID NO: 30 (Exemplary B19 Construct 2) showed higher virion potency relative to recombinant virions comprising an exemplary erythroparvovirus B19 VP1 capsid protein encoded by a nucleotide sequence according to SEQ ID NOs: 29, 31, and 32 (Exemplary B19 Construct 1, Exemplary B19 Construct 3, Exemplary B19 Construct 4, respectively). As shown in FIG. 21, about 60% of K562 cells infected with recombinant virions comprising an exemplary erythroparvovirus B19 VP1 capsid protein encoded by a nucleotide sequence according to SEQ ID NO: 30 (Exemplary B19 Construct 2) showed expression of a heterologous nucleic acid encoding GFP.

It is an insight of the present Example that other cell types can be transduced using exemplary compositions, preparations, nucleotide sequences, recombinant virions, and population of cells comprising recombinant virions described herein.

It is an insight of the present Example that other recombinant virions comprising an erythroparvovirus capsid and be used according to embodiments of the present disclosure.

Accordingly, the present Example confirms that compositions, preparations, nucleotide sequences, recombinant virions, and population of cells comprising recombinant virions comprising an erythroparvovirus VP1 capsid protein encoded by a nucleotide sequence as described herein transduce human cells.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

What is claimed is:

1. A recombinant virion, comprising (1) at least one capsid protein or a variant thereof, of erythroparvovirus or a genotypic variant thereof; and (2) a nucleic acid, wherein the nucleic acid comprises a heterologous nucleic acid,
optionally wherein the erythroparvovirus is selected from primate erythroparvovirus 1 (human erythroparvovirus B19), primate erythroparvovirus 4 (pig-tailed macaque parvovirus), primate erythroparvovirus 3 (rhesus macaque parvovirus), primate erythroparvovirus 2 (simian parvovirus), rodent erythroparvovirus 1, and ungulate erythroparvovirus 1.

2. The recombinant virion of claim 1, wherein the virion is icosahedral.

3. The recombinant virion of claim 1 or 2, wherein the capsid protein comprises a structural protein VP1 capsid protein, a VP2 capsid protein, or combination thereof.

4. The recombinant virion of claim 3, wherein the VP2 capsid protein is present in excess of the VP1 capsid protein.

5. The recombinant virion of claim 3 or 4, wherein the VP1 capsid protein (i) comprises an amino acid sequence that is at least about 60% identical to SEQ ID NO: 9, and/or (ii) is encoded by a nucleic acid sequence that is at least about 90% identical to any one of SEQ ID NOs: 29-33.

6. The recombinant virion of any one of claims 3-5, wherein the VP2 capsid protein (i) comprises an amino acid sequence that is at least about 60% identical to SEQ ID NO: 11, and/or (ii) is encoded by a nucleic acid sequence that is at least about 90% identical to SEQ ID NO: 34.

7. The recombinant virion of any one of the preceding claims, wherein the heterologous nucleic acid comprises a nucleic acid sequence that is at least about 60% identical to a nucleic acid sequence of a target cell.

8. The recombinant virion of any one of the preceding claims, wherein the heterologous nucleic acid is at least about 60% identical to the nucleic acid of a mammal, preferably wherein the mammal is a human.

9. The recombinant virion of any one of the preceding claims, wherein the heterologous nucleic acid is not operably linked to an erythroparvovirus promoter, optionally a human erythroparvovirus B19 promoter.

10. The recombinant virion of any one of the preceding claims, wherein the nucleic acid comprises at least one inverted terminal repeat (ITR).

11. The recombinant virion of claim 10, wherein the at least one ITR comprises:
(a) a dependoparvovirus ITR,
(b) an AAV ITR, optionally an AAV2 ITR, or
(c) an erythroparvovirus ITR, optionally a human erythroparvovirus B19 ITR.

12. The recombinant virion of any one of the preceding claims, wherein the nucleic acid is deoxyribonucleic acid (DNA).

13. The recombinant virion of claim 12, wherein the DNA is single-stranded or self-complementary duplex.

14. The recombinant virion of any one of the preceding claims, wherein the nucleic acid comprises a Rep protein-dependent origin of replication (ori).

15. The recombinant virion of any one of the preceding claims, wherein the nucleic acid comprises a nucleic acid operably linked to a promoter, optionally placed between two ITRs.

16. The recombinant virion of claim 15, wherein the nucleic acid operably linked to a promoter comprises a heterologous nucleic acid encoding a coding RNA and/or a non-coding RNA.

17. The recombinant virion of claim 16, wherein the heterologous nucleic acid encoding a coding RNA comprises:
(a) a gene encoding a protein or a fragment thereof, preferably a human protein or a fragment thereof;
(b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(c) a nucleic acid encoding a reporter, e.g., luciferase or GFP; or
(d) a nucleic acid encoding a drug resistance protein, e.g., neomycin resistance.

18. The recombinant virion of claim 16 or 17, wherein the heterologous nucleic acid encoding a coding RNA is codon-optimized for expression in a target cell.

19. The recombinant virion of any one of claims 15-18, wherein the nucleic acid operably linked to a promoter comprises a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, cystic fibrosis transmembrane conductance regulator (CFTR), F8 or a fragment thereof (e.g., fragment encoding B-domain deleted polypeptide (e.g., VIII SQ, p-VIII)), and/or Lysosomal storage diseases.

20. The recombinant virion of claim 16, wherein the non-coding RNA comprises lncRNA, miRNA, shRNA, siRNA, antisense RNA, and/or guide RNA.

21. The recombinant virion of any one of claims 15-20, wherein the coding RNA (or the protein translated therefrom) or the non-coding RNA increases or restores the expression of an endogenous gene of a target cell.

22. The recombinant virion of any one of claims 15-21, wherein the coding RNA (or the protein translated therefrom) or the non-coding RNA decreases or eliminates the expression of an endogenous gene of a target cell.

23. The recombinant virion of any one of claims 15-22, wherein the promoter is selected from:
(a) a promoter heterologous to the nucleic acid;
(b) a promoter that facilitates the tissue-specific expression of the nucleic acid, preferably wherein the promoter facilitates hematopoietic cell-specific expression or erythroid lineage-specific expression;
(c) a promoter that facilitates the constitutive expression of the nucleic acid; and
(d) a promoter that is inducibly expressed, optionally in response to a metabolite or small molecule or chemical entity.

24. The recombinant virion of any one of claims 15-23, wherein the promoter is selected from the CMV promoter, β-globin promoter, CAG promoter, AHSP promoter, MND promoter, Wiskott-Aldrich promoter, and PKLR promoter.

25. The recombinant virion of any one of the preceding claims, wherein the nucleic acid comprises a non-coding DNA.

26. The recombinant virion of claim 25, wherein the non-coding DNA comprises:
(a) a transcription regulatory element (e.g., an enhancer, a transcription termination sequence, an untranslated region (5′ or 3′ UTR), a proximal promoter element, a locus control region, a polyadenylation signal sequence), and/or
(b) a translation regulatory element (e.g., Kozak sequence, woodchuck hepatitis virus post-transcriptional regulatory element).

27. The recombinant virion of claim 26, wherein the transcription regulatory element is a locus control region, optionally a β-globin LCR or a DNase hypersensitive site (HS) of β-globin LCR.

28. The recombinant virion of any one of the preceding claims, wherein the nucleic acid comprises a nucleic acid sequence that is at least about 80% identical to the nucleic acid sequence of a genomic safe harbor (GSH) of the target cell.

29. The recombinant virion of claim 28, wherein the nucleic acid that is at least about 80% identical to a GSH is placed 5′ and 3′ to the nucleic acid to be integrated, thereby allowing integration to a specific locus in the target genome by homologous recombination.

30. The recombinant virion of claim 29, wherein the nucleic acid to be integrated is a nucleic acid operably linked to a promoter of any one of claims 19-31.

31. The recombinant virion of any one of claims 28-30, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

32. The recombinant virion of claim 31, wherein the GSH is AAVS1, ROSA26, CCR5, Kif6, Pax5, or an intergenic region of NUPL2.

33. The recombinant virion of any one of the preceding claims, wherein the nucleic acid is integrated into the genome of a target cell upon transduction.

34. The recombinant virion of claim 33, wherein the nucleic acid is integrated into a GSH of the genome of a target cell upon transduction.

35. The recombinant virion of claim 34, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

36. The recombinant virion of claim 35, wherein the GSH is AAVS1, ROSA26, CCR5, Kif6, Pax5, or an intergenic region of NUPL2.

37. The recombinant virion of any one of claims 33-36, wherein the nucleic acid is integrated into the target genome by homologous recombination followed by a DNA break formation induced by an exogenous nuclease.

38. The recombinant virion of claim 37, wherein the nuclease is TALEN, ZFN, a meganuclease, a megaTAL, or a CRISPR endonuclease (e.g., a Cas9 endonuclease or a variant thereof).

39. The recombinant virion of any one of the preceding claims, wherein the nucleic acid comprises a nucleic acid sequence encoding at least one replication protein and capsid protein.

40. The recombinant virion of claim 39, wherein the virion is autonomously replicating.

41. The recombinant virion of any one of the preceding claims, wherein the virion binds and/or transduces (a) a hematopoietic cell and/or (b) a cell expressing erythrocyte P antigen.

42. The recombinant virion of any one of the preceding claims, wherein the virion binds and/or transduces (a) an erythroid lineage cell, (b) a cancerous erythroid lineage cell, (c) a hematopoietic stem cell (HSC), or (d) a cell expressing CD36 and/or CD34.

43. The recombinant virion of claim 42, wherein the erythroid lineage cell is a megakaryocyte or an erythroid progenitor cell (EPC), optionally a CD36+ EPC.

44. The recombinant virion of any one of claims 1-41, wherein the virion binds and/or transduces a non-erythroid linage cell or a cancerous non-erythroid lineage cell.

45. The recombinant virion of claim 44, wherein the non-erythroid lineage cell is (a) an endothelial cell, optionally a myocardial endothelial cell, or (b) a hepatocyte.

46. The recombinant virion of any one of the preceding claims, wherein the virion transduces a cell in an erythrocyte P antigen-dependent manner.

47. A pharmaceutical composition comprising the recombinant virion of any one of the preceding claims; and a carrier and/or a diluent.

48. A method of preventing or treating a disease, comprising:
administering to a subject in need thereof an effective amount of the recombinant virion or pharmaceutical composition of any one of claims 1-47.

49. A method of preventing or treating a disease, comprising:
(a) obtaining a plurality of cells;
(b) transducing the cells with the recombinant virion or pharmaceutical composition of any one of claims 1-47, optionally further selecting or screening for the transduced cells; and
(c) administering an effective amount of the transduced cells to a subject in need thereof.

50. The method of claim 48 or 49, wherein the nucleic acid encodes a protein.

51. The method of claim 48 or 49, wherein the nucleic acid decreases or eliminates the expression of an endogenous gene.

52. The method of any one of claims 48-51, wherein the recombinant virion comprises a nucleic acid that encodes a hemoglobin subunit.

53. The method of any one of claims 49-52, wherein the cells are erythroid-lineage cells or bone marrow cells.

54. The method of any one of claims 49-53, wherein the cells are autologous or allogeneic to the subject.

55. The method of any one of claims 48-54, wherein the disease is selected from endothelial dysfunction, cystic fibrosis, cardiovascular disease, diabetes, renal disease, cancer, hemoglobinopathy, anemia, hemophilia, myeloproliferative disorder, coagulopathy, and hemochromatosis.

56. The method of any one of claims 48-55, wherein the disease is selected from sickle cell disease, alpha-thalassemia, beta-thalassemia, hemophilia A, Fanconi anemia, cystic fibrosis, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), and hematologic cancer.

57. The method of any one of claims 48-56, wherein the method further comprises re-administering at least one additional amount of the virion, pharmaceutical composition, or transduced cells.

58. The method of claim 57, wherein said re-administering the at least one additional amount is performed after an attenuation in the prevention or treatment subsequent to said administering the effective amount of the virion, pharmaceutical composition, or transduced cells.

59. The method of claim 57 or 58, wherein the at least one additional amount is the same as the said effective amount.

60. The method of claim 57 or 58, wherein the method further comprises increasing or decreasing the at least one additional amount as compared to the said effective amount.

61. The method of claim 60, wherein the at least one additional amount is increased or decreased based on the expression of an endogenous gene and/or the nucleic acid of the recombinant virion.

62. A method of modulating (i) gene expression, or (ii) function and/or structure of a protein in a cell, the method comprising transducing the cell with the virion or pharmaceutical composition of any one of claims 1-47 comprising a nucleic acid that modulates the gene expression, or the function and/or structure of the protein in the cell.

63. The method of claim 62, wherein the nucleic acid comprises the sequence encoding CRISPRi or CRISPRa agents.

64. The method of claim 62 or 63, wherein the gene expression, or the function and/or structure of the protein is increased or restored.

65. The method of claim 62 or 63, wherein the gene expression, or the function and/or structure of the protein is decreased or eliminated.

66. A method of integrating a heterologous nucleic acid into a GSH in a cell, comprising
(a) transducing the cell with one or more virions or pharmaceutical composition according to any one of claims 1-47 comprising a heterologous nucleic acid flanked at the 5′ end and 3′ end by a donor nucleic acid sequence that is at least about 80% identical to the target GSH nucleic acid; or
(b) transducing the cell with one or more virions or pharmaceutical composition according to any one of claims 1-47 comprising (i) a heterologous nucleic acid flanked at the 5′ end and 3′ end by a donor nucleic acid sequence that is at least about 80% identical to the target GSH nucleic acid, and (ii) a nucleic acid encoding a nuclease (e.g., Cas9 or a variant thereof, ZFN, TALEN) and/or a guide RNA, wherein the nuclease or the nuclease/gRNA complex makes a DNA break at the GSH, which is repaired using the donor nucleic acid, thereby integrating a heterologous nucleic acid at GSH.

67. The method of claim 66, wherein (i) the heterologous nucleic acid flanked by a donor nucleic acid that is at least about 80% identical to the target GSH nucleic acid is transduced in one virion, and (ii) the nucleic acid encoding a nuclease and/or the gRNA are transduced in a separate virion.

68. The method of claim 66 or 67, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

69. The method of any one of claims 66-68, wherein the GSH is AAVS1, ROSA26, CCR5, Kif6, Pax5, or an intergenic region of NUPL2.

70. A method of producing a recombinant virion according to any one of claims 1-46, comprising:
(1) providing at least one vector comprising
(i) a nucleotide sequence comprising at least one ITR nucleotide sequence, optionally further comprising a heterologous nucleic acid operably linked to a promoter for expression in a target cell,
(ii) a nucleotide sequence comprising at least one gene encoding an erythroparvovirus (e.g., B19) VP1 capsid protein and/or a VP2 capsid protein of the recombinant virion of any one of claims 1-46 that is operably linked to at least one expression control sequence for expression in a host cell (e.g., an insect cell, e.g., a mammalian cell), and
(iii) a nucleotide sequence comprising
(A) at least one replication protein of erythroparvovirus (e.g., B19) operably linked to at least one expression control sequence for expression in a host cell,
(B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or
(C) a combination of (A) and (B),
(2) introducing said at least one vector into a host cell, and
(3) maintaining said host cell under conditions such that a recombinant virion according to any one of claims 1-46 is produced.

71. The method of claim 70, wherein two vectors are provided,
(a) a first vector comprising a nucleotide sequence comprising at least one ITR nucleotide sequence, optionally further comprising a heterologous nucleic acid operably linked to a promoter for expression in a target cell, and
(b) a second vector comprising
(i) a nucleotide sequence comprising at least one gene encoding the erythroparvovirus (e.g., B19) VP1 capsid protein and/or VP2 capsid protein of the recombinant virion of any one of claims 1-46 that is operably linked to at least one expression control sequence for expression in a host cell, and
(ii) a nucleotide sequence comprising
(A) at least one replication protein of erythroparvovirus (e.g., B19) operably linked to at least one expression control sequence for expression in a host cell,
(B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or
(C) a combination of (A) and (B).

72. The method of claim 70, wherein three vectors are provided,
(a) a first vector comprising a nucleotide sequence comprising at least one ITR nucleotide sequence, optionally further comprising a heterologous nucleic acid operably linked to a promoter for expression in a target cell,
(b) a second vector comprising a nucleotide sequence comprising a gene encoding the erythroparvovirus (e.g., B19) VP1 capsid protein and/or VP2 capsid protein of the recombinant virion of any one of claims 1-46 that is operably linked to at least one expression control sequence for expression in a host cell, and
(c) a third vector comprising a nucleotide sequence comprising
(A) at least one replication protein of erythroparvovirus (e.g., B19) operably linked to at least one expression control sequence for expression in a host cell,
(B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or
(C) a combination of (A) and (B).

73. A method of producing a recombinant virion according to any one of claims 1-46 in a host cell (e.g., an insect cell, e.g., a mammalian cell), the method comprising:
(1) providing a host cell comprising
(i) a nucleotide sequence comprising at least one ITR nucleotide sequence, optionally further comprising a heterologous nucleic acid operably linked to a promoter for expression in a target cell,
(ii) a nucleotide sequence comprising at least one gene encoding erythroparvovirus (e.g., B19) VP1 capsid protein and/or VP2 capsid protein of the recombinant virion of any one of claims 1-46 that is operably linked to at least one expression control sequence for expression in a host cell, and
(iii) a nucleotide sequence comprising
(A) at least one replication protein of erythroparvovirus (e.g., B19) operably linked to at least one expression control sequence for expression in a host cell,
(B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or
(C) a combination of (A) and (B),
optionally, at least one vector,
wherein at least one of (i), (ii), (iii) (A), (iii) (B), and (iii) (C) is/are stably integrated in the host cell genome, and the at least one vector, when present, comprises the remainder of the (i), (ii), (iii) (A), (iii) (B), and (iii) (C) nucleotide sequences which is/are not stably integrated in the host cell genome, and
(2) maintaining the host cell under conditions such that the recombinant virion is produced.

74. The method of any one of claims 70-73, wherein the at least one replication protein of is an NS1 protein of the erythroparvovirus (e.g., the human erythroparvovirus B19) or a genotypic variant thereof.

75. The method of any one of claims 70-74, wherein the host cell is derived from a species of lepidoptera.

76. The method of claim 75, wherein the species of lepidoptera is Spodoptera frugiperda, Spodoptera littoralis, Spodoptera exigua, or Trichoplusia ni.

77. The method of any one of claims 70-76, wherein the host cell is Sf9.

78. The method of any one of claims 70-77, wherein the at least one vector is a baculoviral vector, a viral vector, or a plasmid.

79. The method of any one of claims 70-78, wherein the at least one vector is a baculoviral vector.

80. The method of any one of claims 70-79, wherein the VP1 capsid protein (i) comprises an amino acid sequence that is at least about 60% identical to the SEQ ID NO: 9, and/or (ii) is encoded by a nucleic acid sequence that is at least about 90% identical to any one of SEQ ID NOs: 29-33.

81. The method of any one of claims 70-80, wherein the VP2 capsid protein (i) comprises an amino acid sequence that is at least about 60% identical to the SEQ ID NO: 11, and/or (ii) is encoded by a nucleic acid sequence that is at least about 90% identical to SEQ ID NO: 34.

82. The method of any one of claims 70-81, wherein the at least one ITR comprises:
(a) a dependoparvovirus ITR,
(b) an AAV ITR, optionally an AAV2 ITR, or
(c) an erythroparvovirus, optionally a human erythroparvovirus B19 ITR.

83. The method of any one of claims 70-82, wherein the at least one expression control sequence for expression in a host cell comprises:
(a) a promoter, and/or
(b) a Kozak-like expression control sequence.

84. The method of claim 83, wherein the promoter comprises:
(a) an immediate early promoter of an animal DNA virus,
(b) an immediate early promoter of an insect virus, or
(c) a host cell promoter.

85. The method of claim 84, wherein the animal DNA virus is cytomegalovirus (CMV), erythroparvovirus (e.g., erythroparvovirus B19), or AAV.

86. The method of claim 84, wherein the insect virus is a lepidopteran virus or a baculovirus, optionally wherein the baculovirus is Autographa californica multicapsid nucleopolyhedrovirus (AcMNPV).

87. The method of any one of claims 83, 84, and 86 wherein the promoter is a polyhedrin (polh) or immediately early 1 gene (IE-1) promoter.

88. The method of any one of claims 70-87, wherein the nucleotide sequence comprising at least one replication protein of an AAV comprises a nucleotide sequence encoding Rep52 and/or Rep78.

89. The method of any one of claims 70-88, wherein the AAV is AAV2.

90. A host cell (e.g., an insect cell, e.g., a mammalian cell), comprising at least one vector, comprising:
(i) a nucleotide sequence comprising at least one ITR nucleotide sequence,
(ii) a nucleotide sequence comprising at least one gene encoding erythroparvovirus (e.g., B19) VP1 capsid protein and/or VP2 capsid protein of the recombinant virion of any one of claims 1-46 that is operably linked to at least one expression control sequence for expression in a host cell, and
(iii) a nucleotide sequence comprising
(A) at least one replication protein of erythroparvovirus (e.g., B19) operably linked to at least one expression control sequence for expression in a host cell,
(B) at least one replication protein of an AAV, optionally wherein the at least one replication protein of an AAV comprises (a) a Rep52 or a Rep40 coding sequence operably linked to at least one expression control sequence for expression in a host cell, and/or (b) a Rep78 or a Rep68 coding sequence operably linked to at least one expression control sequence for expression in a host cell, or
(C) a combination of (A) and (B).

91. The host cell of claim 90, wherein at least one of (i), (ii), (iii) (A), (iii) (B), and (iii) (C) is stably integrated in the host cell genome.

92. The host cell of claim 90 or 91, wherein the at least one replication protein is an NS1 protein of the human erythroparvovirus (e.g., B19) or a genotypic variant thereof.

93. The host cell of any one of claims 90-92, wherein the host cell is derived from a species of lepidoptera.

94. The host cell of claim 93, wherein the species of lepidoptera is Spodoptera frugiperda, Spodoptera littoralis, Spodoptera exigua, or Trichoplusia ni.

95. The host cell of any one of claims 90-94, wherein the host cell is Sf9.

96. The host cell of any one of claims 90-95, wherein the at least one vector is a baculoviral vector, a viral vector, or a plasmid.

97. The host cell of any one of claims 90-96, wherein the at least one vector is a baculoviral vector.

98. The host cell of any one of claims 90-97, wherein the VP1 capsid protein comprises an amino acid sequence that is at least about 60% identical to the SEQ ID NO: 9.

99. The host cell of any one of claims 90-98, wherein the VP2 capsid protein comprises an amino acid sequence that is at least about 60% identical to the SEQ ID NO: 11.

100. The host cell of any one of claims 90-99, wherein the at least one ITR comprises:
(a) a dependoparvovirus ITR,
(b) an AAV ITR, optionally an AAV2 ITR, or
(c) an erythroparvovirus ITR, optionally a human erythroparvovirus B19 ITR.

101. The host cell of any one of claims 90-100, wherein the at least one expression control sequence for expression in a host cell comprises:
(a) a promoter, and/or
(b) a Kozak-like expression control sequence.

102. The host cell of claim 101, wherein the promoter comprises:
(a) an immediate early promoter of an animal DNA virus,
(b) an immediate early promoter of an insect virus, or
(c) a host cell promoter.

103. The host cell of claim 102, wherein the animal DNA virus is cytomegalovirus (CMV), erythroparvovirus (e.g., erythroparvovirus B19), or AAV.

104. The host cell of claim 102, wherein the insect virus is a lepidopteran virus or a baculovirus, optionally wherein the baculovirus is Autographa californica multicapsid nucleopolyhedrovirus (AcMNPV).

105. The method or the host cell of any one of claims 101, 102, and 104, wherein the promoter is a polyhedrin (polh) or immediately early 1 gene (IE-1) promoter.

106. The host cell of any one of claims 90-105, wherein the nucleotide sequence comprising at least one replication protein of an AAV comprises a nucleotide sequence encoding Rep52 and/or Rep78.

107. The host cell of any one of claims 90-105, wherein the AAV is AAV2.

108. A method of purifying the recombinant virion of any one of claims 1-46, wherein the recombinant virion is purified using an antibody, an antigen-binding fragment of an antibody, or a nanobody that binds the recombinant virion.

109. The method of claim 108, wherein the antibody, an antigen-binding fragment of an antibody, or a nanobody binds a heterologous tag in the VP1 capsid protein or the VP2 capsid protein of the recombinant virion.

110. The recombinant virion of claim 109, wherein the heterologous tag comprises an epitope/tag selected from hemagglutinin, His (e.g., 6×-His), FLAG, E-tag, TK15, Strep-tag II, AU1, AU5, Myc, Glu-Glu, KT3, and IRS.

111. A population of cells (e.g., hematopoietic cells) comprising a recombinant virion of any one of claims 1-46 or a pharmaceutical composition of claim 47.


SEQUENCE LISTING (37 sequences)

SEQ ID NO: 1 - DNA, 159 bp, synthetic construct
  source 1..159: mol_type = other DNA
aggaacccctagatggagttggccactccctctctgcgcgctcgctcgctcactgaggccgggcccgaaacgggcccgtcgggcgacctttggtcgcccggcctcagtgagcgagcgagcgcgcagagagggagtggccaactccatctaggggttcct

SEQ ID NO: 2 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 383 bp, synthetic construct
  source 1..383: mol_type = other DNA
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgccttgacactaagacaagcggcgcgccgcttgatcttagtggcacgtcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 5 - DNA, 383 bp, synthetic construct
  source 1..383: mol_type = other DNA
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgacgtgccactaagatcaagcggcgcgccgcttgtcttagtgtcaaggcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 6 - DNA, 2385 bp, synthetic construct
  source 1..2385: mol_type = other DNA
actcgacgaagacttgatcacccgggggatcctgttaaattgagtaaagaaagtggcaaatggtgggaaagtgatgatgaatttgctaaagctgtgtatcagcaatttgtggaattttatgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactcgctaagctcggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaa

SEQ ID NO: 7 - DNA, 2385 bp, synthetic construct
  source 1..2385: mol_type = other DNA
actcgacgaagacttgatcacccgggggatcctgttaaactgagtaaagaaagtggcaaatggtgggaaagtgatgatgaatttgctaaagctgtgtatcagcaatttgtggaattttatgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactcgctaagctcggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaa

SEQ ID NO: 8 - DNA, 2385 bp, synthetic construct
  source 1..2385: mol_type = other DNA
actcgacgaagacttgatcacccgggggatcctgttaaaacgagtaaagaaagtggcaaatggtgggaaagtgatgatgaatttgctaaagctgtgtatcagcaatttgtggaattttatgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactcgctaagctcggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaa

SEQ ID NO: 9 - protein, 781 aa, synthetic construct
MSKESGKWWESDDKFAKAVYQQFVEFYEKVTGTDLELIQILKDHYNISLDNPLENPSSLFDLVARIKNNLKNSPDLYSHHFQSHGQLSDHPHALSSSSSHAEPRGENAVLSSEDLHKPGQVSVQLPGTNYVGPGNELQAGPPQSAVDSAARIHDFRYSQLAKLGINPYTHWTVADEELLKNIKNETGFQAQVVKDYFTLKGAAAPVAHFQGSLPEVPAYNASEKYPSMTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 10 - DNA, 1665 bp, unidentified
  source 1..1665: mol_type = other DNA; note = Human erythroparvovirus B19 VP2
atgacttcagttaattctgcagaagccagcactggtgcaggaggggggggcagtaatcctgtcaaaagcatgtggagtgagggggccacttttagtgccaactctgtgacttgtacattttctagacagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtatactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaa

SEQ ID NO: 11 - protein, 554 aa, unidentified
  source 1..554: note = Human erythroparvovirus B19 VP2
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 12 - DNA, 2172 bp, unidentified
  source 1..2172: mol_type = other DNA; note = Human erythroparvovirus B19
tccggaatattaatagatcatggagataattaaaatgataaccatctcgcaaataaataagtattttactgttttcgtaacagttttgtaataaaaaaacctataaatattccggattattcataccgtcccaccatcgggcgcggatctgccgccatggagctatttagaggggtgcttcaagtttcttctaatgttctggactgtgctaacgataactggtggtgctctttactagatttagacacttctgactgggaaccactaactcatactaacagactaatggcaatatacttaagcagtgtggcttctaagcttgaccttaccggggggccactagcagggtgcttgtacttttttcaagcagaatgtaacaaatttgaagaaggctatcatattcatgtggttattggggggccagggttaaaccccagaaacctcacagtgtgtgtagaggggttatttaataatgtactttatcactttgtaactgaaaatgtgaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgatactagtagcgatgctggggagtctagcggcacaggggcagaggttgtgccatttaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctactagcacatttttattgcatacagactttgagcaggttatgtgtattaaagacaataaaattgttaaattgttactttgtcaaaactatgaccccctattggtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcaggaaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgcatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagcccagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcgatgtagctgccatgtgggagcttctaatcccttttctgtgctaacctgcaaaaaatgtgcttacctgtctggattgcaaagctttgtagattatgagtaa

SEQ ID NO: 13 - protein, 671 aa, unidentified
  source 1..671: note = Human erythroparvovirus B19 Non-structural protein NS1
MELFRGVLQVSSNVLDCANDNWWCSLLDLDTSDWEPLTHTNRLMAIYLSSVASKLDLTGGPLAGCLYFFQAECNKFEEGYHIHVVIGGPGLNPRNLTVCVEGLFNNVLYHFVTENVKLKFLPGMTTKGKYFRDGEQFIENYLMKKIPLNVVWCVTNIDGYIDTCISATFRRGACHAKKPRITTAINDTSSDAGESSGTGAEVVPFNGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFFNLITPGAWNTETPRSSTPIPGTSSGESFVGSPVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 14 - protein, 818 aa, unidentified
  source 1..818: note = Primate Erythroparvovirus 2 VP1
MSEPASKKQKWWDEENAYSDAWLSEFKDIIKDAISLIGEEEEVPVLALKFLQSHLKLDLKYGVDALSGSDALRFLTEHALPLNAVNKDTKGDSTLGIYLKQHLQDYIDNPDKYTLDLSHGPLPDFRETEAEHKSFNEPRGDDAVLTKEDLHEGGGVSLTLPFSNYIGPGNQLQAGNPQSVVDAAARIHDFRYSELIKLGINPYTHWSVADDELLHNIKNEEGFQAQVVRDFFTLKGLFTSTAHFKGELPAVPEYSASENYPNMASVTSTEGTTGAGGGGSNPVHGVWREGAVFSDSSVTCTFSRVFVVPYTAEHAYRVFSPPAENCHSAATGESKVCAVSPVMAYATPWHYIDVNCASLYFSPLEFQRLLENYGSIKPSSMSVTLSEVCIKDVTDKPGGGVQVTDSTTGKLCFLVDDEYQFPYVLGQGQDTLAPELPIWTYLLPQYAYLTVGEVNTKGLTSSTRKQPSEESAFYVLEHANCLLLGTGSSISTAYTFPPLTAESLEGASQHFYEMYNPLYSSRLAVPSALGGQPKVRFVQPTDHAIQPQNFMPGPLVNTVTTAEGDSSSTGAAKALTGISTGSSQNTRISFRPGPRSQPYHYYDEINQKYINGIDSISYGVTTFGNTAKPQEASQAVGRYPNDKEQSKQLQGLDIKTFYSNKGDQKYTEEINRPLMVGSIWNRRAFHYETQLWTKLPNLDEGFKTEFSALGGWALPKPPPMIFLKMQPAPGPEGFASITNSTLAQYATGVLTVTLTFALGPRKHTGRWNPQPACIPPHAAGHLPYILYDTEVTKNSQNHRHGYEKPEECWSAKKRVHPL

SEQ ID NO: 15 - protein, 556 aa, unidentified
  source 1..556: note = Primate Erythroparvovirus 2 VP2
MASVTSTEGTTGAGGGGSNPVHGVWREGAVFSDSSVTCTFSRVFVVPYTAEHAYRVFSPPAENCHSAATGESKVCAVSPVMAYATPWHYIDVNCASLYFSPLEFQRLLENYGSIKPSSMSVTLSEVCIKDVTDKPGGGVQVTDSTTGKLCFLVDDEYQFPYVLGQGQDTLAPELPIWTYLLPQYAYLTVGEVNTKGLTSSTRKQPSEESAFYVLEHANCLLLGTGSSISTAYTFPPLTAESLEGASQHFYEMYNPLYSSRLAVPSALGGQPKVRFVQPTDHAIQPQNFMPGPLVNTVTTAEGDSSSTGAAKALTGISTGSSQNTRISFRPGPRSQPYHYYDEINQKYINGIDSISYGVTTFGNTAKPQEASQAVGRYPNDKEQSKQLQGLDIKTFYSNKGDQKYTEEINRPLMVGSIWNRRAFHYETQLWTKLPNLDEGFKTEFSALGGWALPKPPPMIFLKMQPAPGPEGFASITNSTLAQYATGVLTVTLTFALGPRKHTGRWNPQPACIPPHAAGHLPYILYDTEVTKNSQNHRHGYEKPEECWSAKKRVHPL

SEQ ID NO: 16 - protein, 687 aa, unidentified
  source 1..687: note = Primate Erythroparvovirus 2 NS1
MEMYRGVIQVNANFTDFANDNWWCCFFQLDVDDWPELRGPERLMAHYICKVAALLDTPSGPFLGCKYFLQVEGNHFDNGFHIHVVIGGPFLTPRNVCSAVEGGFNKVLADFTSPTITVQFKPAVSKKGKYHRDGFDFVTYYLMPKLYPNVIYSVTNLEEYQYVCNSLCYRRTMHKRQQPCNGGSVEQSSVSLYSDGEPANKKSKVVTVRGEKFCSLVDSLIERNIFNENKWKETDFKEYAALSASVAGVHQIKTALTLAVSKCNSPAYLGEILTRPNTINFNIRENRIANIFLSNNYCPLYAGKMFLAWVQKQLGKRNTIWLFGPPSTGKTNIAMSLASAVPTYGMVNWNNENFPFNDVPYKSIILWDEGLIKSTVVEAAKSILGGQPCRVDQKNKGSVEVSGTPVLITSNSDMTRVVCGNTVTLVHQRALKDRMVRFDLTVRCSNALGLIPADEAKQWLWWAQNNACDAFTQWHLSSDHVAWKVDRTTLCHDFQSEPEPDSELPSSGESVESFDRSDLSTSWLDVQDQSSSPENSDVEWDIADLLSNEHWIDDLQEDSCSPPRCSTPVAVAEPVEVPTGTGGGLKWEKNYSVHDTNELRWPMFSVDWVWGTNVKRPVCCLEHDKEFGVHCSLCLSLEVLPMLIEKSILVPDTLRCSAHGDCTNPFDVLTCKKCRDLSGLMSFLEHE

SEQ ID NO: 17 - protein, 820 aa, unidentified
  source 1..820: note = Primate Erythroparvovirus 3 VP
MTSSPAKKKPRKWWDDEDAYSEAWFAEFKDILNDVVFAATSEDEGLVFVLKLLQQYYKLDLTHGLDALSMSDAVDFLTNNALGVSSVNKKSDNTSVLGEYLQKQIENYKNNPNKYTLQLSHGPLPDFRESEAKHESSNEPRGDDAVLSKKDLHEGGGFSVQLPFSHYIGPGNELQAGAPESVVDAAARSHDFRYSELIKLGINPYTQWTVADDELLHNIKNEHGFQAQVVRDYFTLKGLFTSTAHFKGELPAVPQYSSSENYPSMASVTATEGATGSGGGGSNAVQAVWREGAIFTDSSVTCTFSRIFVVPYTAEHAYRFFLLLLKNCHSAATGESKVCAVSPVMGYATPWHYIDVNCASLYFSPLEFQRLIENYGSIKPSSMQVTLSEICIKDVTDKPGGGVQVTDSTTGRLCYLVDDEYQFPYVIGQGQDTLAPELPIWTYLLPQYAYLTVGEVNTKGITSATRKQPSEESAFYVLEHANCLLLGTGSSISSSYQFPSVQAESLEGASQHFYEMYNPLYPSRLAVPSALGGQPKVRFVQPTDHAIQPQNFMPGPLVNTITTADGDSSNTGAAKHLQAFLQGSSQNTRISFRPGPRSQPYHYYDEVNQKYVHGIDSISYGMTTYGFTQKPTEGSQAVGRYPNDKEQNKQLQGLNIKTYFNNKGDQKYTEEINRPLMVGSIWNRRAFHYETQLWTKLPNLDEGFKTEFSALGGWALPKPPPMIFLKMNPAPGPEGFASITSSTLAQYATGILTVTLTFALGPRKHTGRWNPQPACTPPHAAGHLPYVLYDPEVTKNSQNHRHGYEKPEECWSAKKRVHLL

SEQ ID NO: 18 - protein, 683 aa, unidentified
  source 1..683: note = Primate Erythroparvovirus 3 NS
MDMFRGVIQLTANITDFANDSWWCSFLQLDSDDWPELRGVERLVAIFICKVAAVLDNPSGTSLGCKYFLQAEGNHYDAGFHVHIVIGGPFINARNVCNAVETTFNKVLGDLTDPSMSVQFKPAVSKKGEYYRDGFDFVTNYLMPKLYPNVIYSVTNLEEYQYVCNSLCYRKNMHKQHMVSTVDASSSSFMNDMYEPATKRSKSCTVKGEKFRNLVDSLIERNIFSESKWKEVDFNEFARLSASVAGVHQIKTAITLAVSKCNSPDYLFQILTRPSTIHFNIKENRIAQIFLNNNYCPLYAGEVFLFWIQKQLGKRNTVWLYGPPSTGKTNVAMSLASAVPTYGMVNWNNENFPFNDVPYKSLILWDEGLIKSTVVEAAKSILGGQPCRVDQKNKGSVEVTGTPVLITSNSDMTRVVWYTVTLVHQRALKDRMVRFDLTVRCSNALGLIPADEAKQWLWWAQSQPCDAFTQWHQVSEHVAWKADRTGLFHDFSTKPEQESNAKSSGKSNDSFAGSDLANLSWLDVEDTSSSSESDLSGDIAELVSNDNWLQSGCPPTRCSTPVTVVEPKQVSPGTGGGLTKWEKNYSVHQENELAWPMFSVDWVWGSHVKRPVCCVEHDKDLVLPHCNLCLSLEVLPMLIEKSINVPDTLRCSAHGDCTNPFDVLTCKKCRDLSGLMSFLEHDQ

SEQ ID NO: 19 - protein, 785 aa, unidentified
  source 1..785: note = Primate Erythroparvovirus 4 VP
MTDEKPKEKKWWETGDPFREAWYNQFVKIFTDLVGNDLDLAEILWRHYGINLDNPFSNPAALPDLVNRIKKNLKDNPDIYTDSLSHGALPDFRESKAEHEKSNEPRGADAILTSKDLHDGGSISLTLPLTHYIGPGNPLQAGSPTDVVDAAARIHDYRYSELIKLGINPYTHWTVADDELLHNVQNVGGFEAQVVKDFFTLKGLFTSTAHFKGELPPVPSYSATEQYPNMATVTATEGTSGSGGGGSNPVHGVWREGAVFSEDSVSCTFSRVFVVPYAAEHSYRVFSPPAENCHSAAAGESRVCAVSPVMGYATPWHYIDVNCASLYFSPLEFQRLLENYGSIKPSSMSVTLSEICVKDVTDKPGGGVQVTDSTTGRLCFLVDDTYQYPYVLGQGQDTLAPELPIWTYLLPQYAYLTVGDVNTKGITSSSRKQPTEETAFYVLEHSSCMLLGTGSSISTSYAFPELPYESLEGAAQHFYEMYNPLYSSRLAVPSALGGQPKVRFVQPTDHALQPQNFMPGPMVNTVTTKEGDSSNTGAAKALTGFSTGTSQNTRISFRPGPNSQPYHYYDEAEQKYVNSIDSISHGVTTFGDRQKPNEASESVGRYPNDKEQQKQLQALNIKTYYSNKGDQKYTEEINRPLMVGAVWNRRSFHYETQLWTKLPNLDENFMAEFSALGGWALKTPPPMIFLKMQPAPGPEGFSGITNTTLAQYATGTLTVTLTFSLGPRKHTGRWNPQPAVYPPHAAGHLPYVLYDPEVTKTSQTHRHGYEKPEELWSAKKRVHPL

SEQ ID NO: 20 - protein, 672 aa, unidentified
  source 1..672: note = Primate Erythroparvovirus 4 NS
MEMFRGVVHVSANFINFVNDNWWCCFYQLEEDDWPRLQGWERLIAHLIVKVAGEFAVPGGSTLGLQYFLQAEHNHFDEGFHVHVVVGGPFVTPRNVCNIVETGFNKVLRELTEPTYEVSFKPAISKKGKYARDGFDFVTNYLMPKLYPNVVYSVTNFSEYEYVCNSLAYRRNMHKKALTNTADEGEGTSTNSEWGPEPKKQKTGTVRGEKFVSLVDSLIERGIFTENKWKQVDWLKEYACLSGSVAGVHQIKTALTLAISKCNSPEYLCELLTRPSTINFNIKENRICKIFLQNDYDPLYAGKVFLAWLGKELGKRNTIWLFGPPTTGKTNIAMSLATAVPSYGMVNWNNENFPFNDVPHKSIILWDEGLIKSTVVEAAKAILGGQNCRVDQKNKGSVEVQGTPVLITSNNDMTRVVSGNTVTLIHQRALKDRMVEFDLTVRCSNALGLIPAEECKQWLFWSQHTPCDVFSRWKEVCEFVAWKSDRTGICYDFSENEDLPGTQTPLLNSPVTSKTSALKKTIAALATAAVGTLQTSLTNNNWESSEDSGSPPRSSTPLASPERGEVPPGQQWELNTSVNSVNALNWPMYTVDWVWGSKAQRPVCCLEHDTESSVHCSLCLSLEVLPMLIENSINQPDVIRCSAHAECTNPFDVLTCKKCRELSALWSFVKYD

SEQ ID NO: 21 - protein, 829 aa, unidentified
  source 1..829: note = Rodent Erythroparvovirus 1 VP1
MPKRKGAGEAFRVLLDELFGGILSVGGDAFDDPVSELAEHLTLSGIGDADTFKKWQEKDLRHIAQLVAEFETQYNKKELDTLVVDEVKKVANKVVPGLGETGAAVANTAKRLKTDEDPLSFGAPPLTENAPVPVAEPDVAIVSEPNRDTAAEQLERGLAEPDHGGIHLPADRYLGPGNPLENGPPVDPVDAVARIHDFRYADLEKQGINPYTTYTIADEELLKNLEHKTGGRAAIARAFFNFKKLTFPHAHLQGPLPAVKSWKTEQLGLAGMQQASAVSGAGGDHTPAALWAQGAKFSGDSVTCFMTRRCYLPFDEDPTYRAIAHSESDRSNFTKIMVNTGTHTVMGYTTPWHYVDYNNMALFFSPQEFQYLLENYEEIAPKSLTTVLSDLVVKDVSIQDQKTQVTDSGTGGVAIFADESYTYPYVLGNGQRTLPSDIPIQVYELPKYAYLTCGKRTDVGMKGGSLPTHDSDFFFLEHAMFKIYKTGDFFVSPYSFPSLRPRSLMGASQHFFMMQNPLYDYGMDVLTEIGTHGQWSSLDKWEYHGRPQNFFPGPKIPSHVAAEGDRGGKAELQKVATGTSVGDDWYSRYTFRPMPSCQAYSHADPKDPDSDIPVVSIDAVAAGQQSEKPKPPHAKESKFPYKQGRLPNDIEMAKQLQGVNDKMYLVQTLAGQNTTPAQIIPLMPGSVWNERALHYESQIWTKIPNLDKGFMTDHPALGGWGMSTPPPQIFIKMIPTPAPSVEGGGTTSTLHQYAIFNMTVKLEFTLKKRGLAGRWNPQPPVNPPSAVGHLPYVLYDNGQLTGVSSDVQSQNGYERSDELWTAKSRVRHL

SEQ ID NO: 22 - protein, 558 aa, unidentified
  source 1..558: note = Rodent Erythroparvovirus 1 VP2
MQQASAVSGAGGDHTPAALWAQGAKFSGDSVTCFMTRRCYLPFDEDPTYRAIAHSESDRSNFTKIMVNTGTHTVMGYTTPWHYVDYNNMALFFSPQEFQYLLENYEEIAPKSLTTVLSDLVVKDVSIQDQKTQVTDSGTGGVAIFADESYTYPYVLGNGQRTLPSDIPIQVYELPKYAYLTCGKRTDVGMKGGSLPTHDSDFFFLEHAMFKIYKTGDFFVSPYSFPSLRPRSLMGASQHFFMMQNPLYDYGMDVLTEIGTHGQWSSLDKWEYHGRPQNFFPGPKIPSHVAAEGDRGGKAELQKVATGTSVGDDWYSRYTFRPMPSCQAYSHADPKDPDSDIPVVSIDAVAAGQQSEKPKPPHAKESKFPYKQGRLPNDIEMAKQLQGVNDKMYLVQTLAGQNTTPAQIIPLMPGSVWNERALHYESQIWTKIPNLDKGFMTDHPALGGWGMSTPPPQIFIKMIPTPAPSVEGGGTTSTLHQYAIFNMTVKLEFTLKKRGLAGRWNPQPPVNPPSAVGHLPYVLYDNGQLTGVSSDVQSQNGYERSDELWTAKSRVRHL

SEQ ID NO: 23 - protein, 711 aa, unidentified
  source 1..711: note = Rodent Erythroparvovirus 1 NS1
MAQACLSLSWADCFAAVIKLPCPLEEVLSNSQFWQYYVLCKDPLDWPALQVTELAHGWEVGAYCAFADALYLYLVGRLADEFSAYLLFFQLEPGVENPHIHVVAQATQLSAFNWRRILTQACHDMALGFLKPDYLGWAKNCVNIKKDKSGRILRSDWQFVETYLLPKVPLSKVWYAWTNKPEFEPIALSAAARDRLMRGNALCNQPGPGPSFGDRAEIQGPPIKKTKASDEFYTLCHWLAQEGILTEPAWRQRDLDGYVRMHTSTQGRQQVVSALAMAKNIILDSIPNSVFATKAEVVTELCFESNRCVRLLRTQGYDPVQFGCWVLRWLDRKTGKKNTIWFYGVATTGKTNLANAIAHSLPCYGCVNWTNENFPFNDAPDKCVLFWDEGRVTAKIVESVKAVLGGQDIRVDQKCKGSSFLRATPVIITSNGDMTVVRDGNTTTFAHRPAFKDRMVRLNFDVRLPNDFGLITPTEVREWLRYCKEQGDDYEFPDQMYQFPRDVVSVPAPPALPQPGPVTNAPEEEILDLLTQTNFVTQPGLSIEPAVGPEEEPDVADLGGSPAPAVSSTTESSADEDEDDDTSSSGDHRGGGGGVMGDLHASSSSFFTSSDSGLPTSVNTSDTPFSFSPVPVHHHGPPTLLPTSRPTRDLARGRPSFRQYEPLKGRCADSTTFGRPSWAAPCAVYNTAELTRRGAGVRVVKGSRPGAISGK

SEQ ID NO: 24 - protein, 143 aa, unidentified
  source 1..143: note = Rodent Erythroparvovirus 1 NS2
MPRKKRSLISLPKQTSSLNLGSLLSRPLDLKKNLMSQILEGLQHQQSAAPQSPVPTRTRTTTPPPLATTEEEEEGSWEIYTLLLPPSLLPVTQDSPLPSTPATPLSPSAPYQCTTTDPQRFSRPHARHAIWPVGARLSASTSH

SEQ ID NO: 25 - protein, 947 aa, unidentified
  source 1..947: note = Ungulate Erythroparvovirus 1 VP
MAFNPLTMSSRLLVPVTPVSKLDLLKKKWFAFPDVSKILLEALSHSGFGDPKKWKEADADIIEALLDEALRLGPRLEKPAWFYDLQRAIGLARFSASLEQTVFLNEMLIKLTRGPVVPKYPEPDIVIRDPAPLTPEVEAPTSTPENSPDQSSVASDPVEMEEGSSTPIPDPVPQSEDMETEETTIPDQPPPPSPQIVDEVEDMAMGVEDLSIVEDASEQHQSPAGEPTPDITSSVGNRDDESREESREADLQDLSAGLGAAGGSAIAALGSGLIPAATVATAYPRPDQFLRDYLARYDQMYPSGSRYPPRWEQLKSLYDKGMTVKEVWDLLNKNSNNSNLQAKDTDKKQTAPSSSSAPQESAAAMASGDKSGVNPSGGSAPLSATVWASGAQFEADHVITHMSRTVFIPFQQAHRYEPIVWRGRRTADGWLSFWPDHPVIGYKTPWFYLDVNAINRHFSPGEWQEVLERYGSIVPESMEIILSDFCIKDVSVVDGKTTVTDSSTGGVCVFVDDGYKFPYVLGHSQNTLPGPLPTDIYSPPQYAYLTTGKKTKVAAYASGEGPMPMDSIAIPSQETAFYVLENSFYTIQRAGGGFAHSYNFPSLKPISLEGFSQHWMLMDNPLYPSRLWVPEKVGGASKWGAVKNDDYGKKPLNWMPGPNIPSHTIEQSDQAGQRVELDRDVEGQKVWTGTSFGSRPENRWSMRPLGVNQPYAYDAYEDETDKIVTVDAIGYGTAKASAALGQDTGEVPENASVGRVPDDTECNKQGGGGNHLFQVKSLAHNNFTEQMKNQTVPLMPGSVWQNRALHYESQIWAKIPNVDGEFMCERPALGGWGMHDPPPQIFMKMQPVPAPKSLNSTTEAGFPSEHYLHQYAYCVMTVRMRWKTTTRTGPTRWNPQPTFGPPEATDHIPYILYDRLSTIHKTRGQFTNAYYEEPESVWTARGRVRHL

SEQ ID NO: 26 - protein, 651 aa, unidentified
  source 1..651: note = Ungulate Erythroparvovirus 1 NS
MESYSRAVIRLPWENIYEAIQEAAWPSLAAVEPQRPGDLPYDWPLLYDEDRRYVVACDALWSILQRRAAVFGRWAGYLQLEPSQAGGPGRHLHLLLSAPGIRGRSWTAFLRNAVAEWARTTVHLNYVDAIDIPRNTHGRILEADADFVFRYLAPKLPLREVTWAWTNEDQFKPFALCEPKRRELIQRATTQDRANGLDGPPAKRSRAADEFHQLVHFLADKGIVDPDKWMALFPDSYITWSSSAQGRQQVNSACELALQIILTRGVLSRFLAPNPSNIFPENNRAVELLRMQGHDPVSFGQLVLAWADKQLGKRNTLWFWGPPSTGKTNLALAIARALPRFGMVNWTNENFPFNDAPHKCVLVWDEGRITAKIVEAVKSILGGQAVRVDQKCKGSVSLSPTPVLITSNADIRYVRDGNIVTGDHVKALSERMVIVHFSTPCPANFGLLKAEEIVDWLNYVKSCPGSITADTVQATWGTRSAPNLFEIKRKAPQTASPLGPQAEEQEEAAAYRCPSSPASSRSSSPDIFGITKSPAPLEDLSSDSSSECSLPFTPSNAAWFTPMPPARPLQPPLFGVDWIYSTQWKQPVCCLDHETEPCNLCIDIAERCVLFRVSEPDLLRCPDHRHEENPFDVLLCRHCQALSGLETLQSA

SEQ ID NO: 27 - DNA, 1667 bp, Homo sapiens
  source 1..1667: mol_type = other DNA
cccagcaatggatcgatgcacggctgtcggggccgacaggctgacctttactgagctcaggttttcatctccctgttgggagcccaggaaggtcttgctgtggagagaggaacggtgagaagccttggcctgcgagggggagaggcttggcgtgggtgcagtgaagacagcttctgagagctgaaagcccttggaggtcacttatttcaatttcttccaaacacaccacttttacagatgagaaaactgagacttggtgagaaatgacttgtccaaggtcactctaagaggctttgacacagctccagaatccagtgtgtgtgtatgtgtgtgtgtacacatccaacatacatacatctgtatattataaatattatacatattattacgtatacatacacagatacattatatgcatgtgtacgtgtatatcgggagtgtctatacatgtgtattatgaaagcctggctgtggctacgtgtgatgccgtgcctgcgctcactctggtcgtcaacagtttggtcccgcaacatcccgggtagccgccgatccctgagccaccaggcatttcatgcagttctgcaaagccatggagaggagctgaagaaacctcatggtccttttcaaatcgtttcttcctcctcctcctctgcaaagattttctctaagcccagtttgaatccttcagaaacagaacttggctgcgaagtcactttgaaagactttccatatgttaattgcagccggccaaggtctggagcagaggtgggagcccaccatctgcagacggggtcggcccccagtgcgctctgcaaatccccgtcatctggcaggtgtcgttttgggttaattaagagctatactgagcccgtttacctgtcacttctgagaattttaggaaactttgactttcttgccatctctgagctttgagcgaaggggaagctgaaaacacctctgaatctggtgatgtttctgcctctgggatctccaggacagctgcattaagtgcatcttatcataacccctttttaaactttttattttaatcagtgttctctagttagtgcattggtttttacagtcacgtcttctatattggaagacagtactgtttgggggaaacccaccatttgtctgaaatttcttaaggctctgctttctctctgtgtctttgaggaaacagcatacattcctctagctttgttctgtgtaatggctttggagaaactttgaatttgcaggtcaggggctctttcacccattggggtttggggctgtcagtgctaacctcagagctctatgttcatggagggatgactcagttacatccccagatagctgggttctcggttggtcaataggcccccttcttcagtatgagagaattttctctctgtgctgttgacaatgttctattaatatatcttggtaggggtttgggtcacacagatctatgcatttgtcaaaacacagcaatagcacatttaagatttgtgtgtttcattatgtgtaaattttgtatccaaagaaaaaactagtaaacaagtaatgaacttcagttaattgtatgcatgctgaagtacttaggggaaagtgtactgatgtttgcatttacttggaaatgaaatacacattaaggtgaaagaaaggctagagggatgaag

SEQ ID NO: 28 - DNA, 1637 bp, Homo sapiens
  source 1..1637: mol_type = other DNA
agtggtgtgatcatggcttactgcagcctcaacctcccagattcaagtgatcctcctgcctcagcctaccgagtagctgggactacagatgcatgccatcacgcctgactaattttaccttttgtagagatgaggtccctctgtgttgccaaaggtggtctagaactcctgggctcaaatgatcctcccccctccctgggccttccaaagtactgggattacaggtgtaagccaatgcactcagccccatgttacttaatagaaaggtttttcttcccctttttcctgcaccctttgctgctctcacggggaatttctagcatctctaagctctggtctccagtctgaggaagttgtgctgcctgtatgtgacaagagaaataagatgttggcacatgaataggatgttcgccctttggtgaactagagcatgtgagccaattcttaagccagatttttcagcagagaacaattgcaattcacaatcacattttccaggcatgactcatccctatagtatacaataatatgaagagaggctggaaaccccatgcttggcaaataccagtgcccaggcactgcaagctttcttttgtggcagatttttcatacaaactgagtccatcagtctcagagtcccattcaataacaaaagaagaaataaatggggagattaactgctattggaaatgaaggtgttgaaaatgtaaactaaacaaagcaaagcaccccttcactcagttggatccttctaacatagaatcaaacagccatctaaaaccaacaggaaaaccggaccgagggtggagagaaaccgtgtggcaccatcaggaggtaactcccatggtgaggaggagtttcctggctcccattagactttcattttaacccttatgatttttccttttttttttttttttgtatttacatcttttctgctgaactaaaatgataaactctgagagcagagattagattagaatcttctcagcctctccacaattttaaaagcagtgctggccacaggaaaaaaaaaaaaaggtactcaaaaaacactttttttgtttgtgaatgacaatttgaaattgactttgagaaatcttggcagccaagaaaatggctggagaagactttacagcttccgagaagtaggaggatgcagcaggcttctggagggtcaggggaggagctgatcaactggaggcgggagagggaggccatagtgggagagatgaaacgggaaaggaatactagcattttttaaaaagcataaggggaacaaagggtggatctttattacaataaagtggaggcagccagggtacaaggtacaagtttatggaggaaaaaaatggcaaaatataggcccagtcttctgtcctcctctctgacagggaagggtattggatgttcactctatgaaaaagcaacatattaagttagttgttctagacaagaaaagtaggaaagatattgtaggaaccctttgccctcaaacacatattggcccaccattctcagaaggcaatctcagctggcatgacagagcatctggttgcagaggctcttggggactgagtggctgctgaacgaacaccagcccctctctttggcccatgggtaaaagcagccactgc

SEQ ID NO: 29 - DNA, 2768 bp, synthetic construct
  source 1..2768: mol_type = other DNA
atcatggagataattaaaatgataaccatctcgcaaataaataagtattttactgttttcgtaacagttttgtaataaaaaaacctataaatattccggattattcataccgtcccaccatcgggcgcggatctgccaccacgagtaaagaaagtggcaagtggtgggaaagtgacgacgaatttgctaaagctgtgtatcagcaatttgtggaattttacgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcacggacagttatctgaccacccccacgccttatcatccagtagcagtcacgcagaacctagaggagaagacgcagtattatctagtgaagacttacacaagcctgggcaagttagcgtacaactacccggtactaactacgttgggcctggcaacgagctacaagctgggcccccgcaaagtgctgttgacagtgctgcaaggattcacgactttaggtatagccaactcgctaagctcggaataaatccatatactcattggactgtagcagacgaagagcttttaaaaaatataaaaaacgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 30 - DNA, 2768 bp, synthetic construct
  source 1..2768: mol_type = other DNA
atcatggagataattaaaatgataaccatctcgcaaataaataagtattttactgttttcgtaacagttttgtaataaaaaaacctataaatattccggattattcataccgtcccaccatcgggcgcggatctgccaccctgagtaaagaaagtggcaagtggtgggaaagtgacgacgaatttgctaaagctgtgtatcagcaatttgtggaattttacgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcacggacagttatctgaccacccccacgccttatcatccagtagcagtcacgcagaacctagaggagaagacgcagtattatctagtgaagacttacacaagcctgggcaagttagcgtacaactacccggtactaactacgttgggcctggcaacgagctacaagctgggcccccgcaaagtgctgttgacagtgctgcaaggattcacgactttaggtatagccaactcgctaagctcggaataaatccatatactcattggactgtagcagacgaagagcttttaaaaaatataaaaaacgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 31 - DNA, 2768 bp, synthetic construct
  source 1..2768: mol_type = other DNA
atcatggagataattaaaatgataaccatctcgcaaataaataagtattttactgttttcgtaacagttttgtaataaaaaaacctataaatattccggattattcataccgtcccaccatcgggcgcggatctgccaccttgagtaaagaaagtggcaagtggtgggaaagtgacgacgaatttgctaaagctgtgtatcagcaatttgtggaattttacgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcacggacagttatctgaccacccccacgccttatcatccagtagcagtcacgcagaacctagaggagaagacgcagtattatctagtgaagacttacacaagcctgggcaagttagcgtacaactacccggtactaactacgttgggcctggcaacgagctacaagctgggcccccgcaaagtgctgttgacagtgctgcaaggattcacgactttaggtatagccaactcgctaagctcggaataaatccatatactcattggactgtagcagacgaagagcttttaaaaaatataaaaaacgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttccaggcagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtctactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 32 - DNA, 5283 bp, synthetic construct
  source 1..5283: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttggtaccgcatgcctcgagttacaatgggtgcacacggcttttggctgtccacaattcttcaggcttttcatatccatgtctgtggtgttgttttgcatctgtagctgtagggtcatatagtacatatggtaaatgacctgctgcgtgcgggggatatactccaggttgaggattccaccgtcccgtagctttacggggccccaatttaaatgtcatggttactgtcataattcccacggcatactgaactaaggtagtaattcccattgatttaatacctccaattggcccactttgtggtaatatttttaaaaatatttgaggaggtggctgatgcaaaccccatcctcctaaggctgcaaactgagttttaaaactgtcatctaaatttggaattttactccacagctggctttcatagtgaagggctcttctgttccatacagaacccaccattagggggcgctcaatttgatctgtatattgctgggttcctttattgggaaagtaggtgtgcatgtttaaaccctgtaactgttttagctgttctttttcatttggaaatctacccactccttgctgatactctttgtcttcagcgttaccataagtggtctgaccatgagaaatagcatttattcctgtgacatatttatctgtgtcccagtggtggtacggctgagacactggccccgggcgtaaggatattctagtgttttgagaggtacctgtgctaaggcctgttaaggctttcccagctccagtattagagctgtctccctcctttgtagacactgagtttactagtggccctggcatgaagttttggggctgaattgcatggtcttcatgtgttaaagatctaaattttgggtcacctcctaatgtgtcaggaacccctaagcgggatccgtataagggattgtacatctcataaaagtgttgactgcagccctctaaattttctgggggcactggaggaaacttataagacatagttgctgtacctcctgtacctaaaagctgaaaagaactgtgttccaaaacataaaatgctgattcttcacttgctaattttttgctgtctccagaaattccttgtgtgttaacatctcctactgttaagtaagcatattgagggggaaagtagacccaaataggaagttctggggctaaagtatcttgaccttgccctaacacatatgggtacttgtattcatggtctactaacatgcataggcgccctgtagtgctgtcagtaacctgcaccccccctccagttttgtctgtaacatccttaacagcaatttctgatatggttacagttaaagcatcaggagctatacttccataattttcaattaagtgctgaaactctaaaggtgaaaaaaataagtttaaagcattaaaatctaaatatctccatggggttgagtatcccattatgggactaatggtgcaaacctttgcctcctttccactggcattgtggcagctacttgctgcgggagaaaacaccttataatggtgctctgggtcatatggaattaaaaactgcctggaaaatgtacaagtcacagagttggcactaaaagtggccccctcactccacatgcttttgacaggattactgcccccccctcctgcaccagtgctggcttctgcagaattaactgaagtcatgcttgggtatttttctgaggcgttgtaagcgggaacttccggcaaacttccttgaaaatgggccacaggggcagctgcaccttttaaagtaaagtagtcttttactacttgtgcttgaaacccagtttcgttttttatattttttaaaagctcttcgtctgctacagtccaatgagtatatggatttattccgagcttagcgagttggctatacctaaagtcgtgaatccttgcagcactgtcaacagcactttgcgggggcccagcttgtagctcgttgccaggcccaacgtagttagtaccgggtagttgtacgctaacttgcccaggcttgtgtaagtcttcactagataatactgcgtcttctcctctaggttctgcgtgactgctactggatgataaggcgtgggggtggtcagataactgtccgtgactttgaaaatgatgactatataagtctggagaatttttaaggttatttttaatgcgagcaactaagtcaaacagagaggatgggttttctaggggattatctaaagaaatattataatgatcttttaatatttgaataagctctaagtctgttccagtaaccttttcgtaaaattccacaaattgctgatacacagctttagcaaattcgtcgtcactttcccaccacttgccactttctttactcgtatttataggtttttttattacaaaactgttacgaaaacagtaaaatacttatttatttgcgagatggttatcattttaattatctccatgatctattaatattccggagtatacgtatactccggaatattaataggcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcattccggattattcataccgtcccaccatcgggcgcggatctgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgaactccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataagagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 33 - DNA, 3498 bp, synthetic construct
  source 1..3498: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaagcttcctaggccgccaccatgagcaaggagagcggcaagtggtgggagagcgacgacgagttcgccaaggccgtgtaccagcagttcgtggagttctacgagaaggtgaccggcaccgacctggagctgatccagatcctgaaggaccactacaacatcagcctggacaaccccctggagaaccccagcagcctgttcgacctggtggcccgcatcaagaacaacctgaagaacagccccgacctgtacagccaccacttccagagccacggccagctgagcgaccacccccacgccctgagcagcagcagcagccacgccgagccccgcggcgaggacgccgtgctgagcagcgaggacctgcacaagcccggccaggtgagcgtgcagctgcccggcaccaactacgtgggccccggcaacgagctgcaggccggccccccccagagcgccgtggacagcgccgcccgcatccacgacttccgctacagccagctggccaagctgggcatcaacccctacacccactggaccgtggccgacgaggagctgctgaagaacatcaagaacgagaccggcttccaggcccaggtggtgaaggactacttcaccctgaagggcgccgccgcccccgtggcccacttccagggcagcctgcccgaggtgcccgcctacaacgccagcgagaagtaccccagcatgaccagcgtgaacagcgccgaggccagcaccggcgccggcggcggcggcagcaaccccgtgaagagcatgtggagcgagggcgccaccttcagcgccaacagcgtgacctgcaccttcagccgccagttcctgatcccctacgaccccgagcaccactacaaggtgttcagccccgccgccagcagctgccacaacgccagcggcaaggaggccaaggtgtgcaccatcagccccatcatgggctacagcaccccctggcgctacctggacttcaacgccctgaacctgttcttcagccccctggagttccagcacctgatcgagaactacggcagcatcgcccccgacgccctgaccgtgaccatcagcgagatcgccgtgaaggacgtgaccgacaagaccggcggcggcgtgcaggtgaccgacagcaccaccggccgcctgtgcatgctggtggaccacgagtacaagtacccctacgtgctgggccagggccaggacaccctggcccccgagctgcccatctgggtgtacttccccccccagtacgcctacctgaccgtgggcgacgtgaacacccagggcatcagcggcgacagcaagaagctggccagcgaggagagcgccttctacgtgctggagcacagcagcttccagctgctgggcaccggcggcaccgccaccatgagctacaagttcccccccgtgccccccgagaacctggagggctgcagccagcacttctacgagatgtacaaccccctgtacggcagccgcctgggcgtgcccgacaccctgggcggcgaccccaagttccgcagcctgacccacgaggaccacgccatccagccccagaacttcatgcccggccccctggtgaacagcgtgagcaccaaggagggcgacagcagcaacaccggcgccggcaaggccctgaccggcctgagcaccggcaccagccagaacacccgcatcagcctgcgccccggccccgtgagccagccctaccaccactgggacaccgacaagtacgtgaccggcatcaacgccatcagccacggccagaccacctacggcaacgccgaggacaaggagtaccagcagggcgtgggccgcttccccaacgagaaggagcagctgaagcagctgcagggcctgaacatgcacacctacttccccaacaagggcacccagcagtacaccgaccagatcgagcgccccctgatggtgggcagcgtgtggaaccgccgcgccctgcactacgagagccagctgtggagcaagatccccaacctggacgacagcttcaagacccagttcgccgccctgggcggctggggcctgcaccagccccccccccagatcttcctgaagatcctgccccagagcggccccatcggcggcatcaagagcatgggcatcaccaccctggtgcagtacgccgtgggcatcatgaccgtgaccatgaccttcaagctgggcccccgcaaggccaccggccgctggaacccccagcccggcgtgtaccccccccacgccgccggccacctgccctacgtgctgtacgaccccaccgccaccgacgccaagcagcaccaccgccacggctacgagaagcccgaggagctgtggaccgccaagagccgcgtgcaccccctgtaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 34 - DNA, 2810 bp, synthetic construct
  source 1..2810: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggttgaggaacctgttaagatgaccagcgtgaacagcgccgaggccagcaccggcgccggcggcggcggcagcaaccccgtgaagagcatgtggagcgagggcgccaccttcagcgccaacagcgtgacctgcaccttcagccgccagttcctgatcccctacgaccccgagcaccactacaaggtgttcagccccgccgccagcagctgccacaacgccagcggcaaggaggccaaggtgtgcaccatcagccccatcatgggctacagcaccccctggcgctacctggacttcaacgccctgaacctgttcttcagccccctggagttccagcacctgatcgagaactacggcagcatcgcccccgacgccctgaccgtgaccatcagcgagatcgccgtgaaggacgtgaccgacaagaccggcggcggcgtgcaggtgaccgacagcaccaccggccgcctgtgcatgctggtggaccacgagtacaagtacccctacgtgctgggccagggccaggacaccctggcccccgagctgcccatctgggtgtacttccccccccagtacgcctacctgaccgtgggcgacgtgaacacccagggcatcagcggcgacagcaagaagctggccagcgaggagagcgccttctacgtgctggagcacagcagcttccagctgctgggcaccggcggcaccgccaccatgagctacaagttcccccccgtgccccccgagaacctggagggctgcagccagcacttctacgagatgtacaaccccctgtacggcagccgcctgggcgtgcccgacaccctgggcggcgaccccaagttccgcagcctgacccacgaggaccacgccatccagccccagaacttcatgcccggccccctggtgaacagcgtgagcaccaaggagggcgacagcagcaacaccggcgccggcaaggccctgaccggcctgagcaccggcaccagccagaacacccgcatcagcctgcgccccggccccgtgagccagccctaccaccactgggacaccgacaagtacgtgaccggcatcaacgccatcagccacggccagaccacctacggcaacgccgaggacaaggagtaccagcagggcgtgggccgcttccccaacgagaaggagcagctgaagcagctgcagggcctgaacatgcacacctacttccccaacaagggcacccagcagtacaccgaccagatcgagcgccccctgatggtgggcagcgtgtggaaccgccgcgccctgcactacgagagccagctgtggagcaagatccccaacctggacgacagcttcaagacccagttcgccgccctgggcggctggggcctgcaccagccccccccccagatcttcctgaagatcctgccccagagcggccccatcggcggcatcaagagcatgggcatcaccaccctggtgcagtacgccgtgggcatcatgaccgtgaccatgaccttcaagctgggcccccgcaaggccaccggccgctggaacccccagcccggcgtgtaccccccccacgccgccggccacctgccctacgtgctgtacgaccccaccgccaccgacgccaagcagcaccaccgccacggctacgagaagcccgaggagctgtggaccgccaagagccgcgtgcaccccctgtaataactcgagcatgcatctagagatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 35 - DNA, 2633 bp, synthetic construct
  source 1..2633: mol_type = other DNA
atcatggagataattaaaatgataaccatctcgcaaataaataagtattttactgttttcgtaacagttttgtaataaaaaaacctataaatattccggattattcataccgtcccaccatcgggcgcggatctcctgttaagacggctgccgacggttatctacccgattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 36 - DNA, 469 bp, synthetic construct
  source 1..469: mol_type = other DNA
cgaaagtcacacccggaccccgacgccctggtaccttactctccctctcctactgttttgacgaccagaatagattccctctgtctcttctcttttccccgtgtgggtcatccggtgggacaggggtgtcttagggagggggtcttgccggacgagagacgggagtagaggaccgtaaaggagagtaggaaaaaaggactatttaaaagttaggtaagtatgatagaccagtaggtgcacttatctataaaaaaaaaaccggtcagtataccggggtaaaagaaacatgaaatgacttcaatcgagatcacttaggtccctcgtccccgacatcccaccccgacctcggacttctttctgttttccctagtgacactattataccacccccctcccaatgggtcaagactggtgaaaaaaagagacagagttggttctttacgtctcacggaagtggtgagacattgga

SEQ ID NO: 37 - DNA, 42 bp, synthetic construct
  source 1..42: mol_type = other DNA
gcccgggcaaagcccgggcgtcgggcgacctttggtcgcccg